(19) United States
(12) Patent Application Publication
(10) Pub. No.: US 2026/0126446 A1
(43) Pub. Date: May 7, 2026

(54) Uses of Siglec-9 ECD Fusion Molecules in Cancer Treatment

(71) Applicant: Alector LLC, South San Francisco, CA (US)

(72) Inventors: Samuel Nalle, Pacifica, CA (US); Spencer Liang, San Mateo, CA (US)

(73) Assignee: Alector LLC, South San Francisco, CA (US)

(21) Appl. No.: 19/127,501

(22) PCT Filed: Nov. 6, 2023
(86) PCT No.: PCT/US2023/078776

§ 371 Date: May 6, 2025

Related U.S. Application Data
(60) Provisional application No. 63/423,434, filed on Nov. 7, 2022.

(51) Int. Cl. G01N 33/575; G01N 33/569

(52) U.S. Cl. CPC G01N 33/5759; G01N 33/56972

(57) ABSTRACT

The present disclosure relates to methods of treating certain cancers with a Siglec-9 extracellular domain (ECD) or a Siglec-9 ECD fusion molecule, including cancers expressing high levels of CD163 and/or Siglec-9 in tumor or immune cells, and methods of identifying cancers responsive to a Siglec-9 ECD or Siglec-9 ECD fusion molecule, for example by determining the level of CD163 and/or Siglec-9, and in some embodiments also CD68 and/or sialic acid expression in tumor or immune cells.

### CROSS-REFERENCE TO RELATED APPLICATIONS

This application claims the benefit of U.S. Provisional Application No. 63/423,434, filed Nov. 7, 2022, which is hereby incorporated by reference in its entirety.

### SEQUENCE LISTING

The present application is being filed with a Sequence Listing which has been submitted electronically in XML format and is hereby incorporated by reference in its entirety. The Sequence Listing is provided as a file entitled “01209-0016-OOPCT_ST26.xml”, created on Oct. 26, 2023, and is 322,000 bytes in size.

### FIELD

The present disclosure relates to methods of treating certain cancers with a Siglec-9 extracellular domain (ECD) or a Siglec-9 ECD fusion molecule, including cancers expressing high levels of CD163 and/or Siglec-9 in tumor or immune cells, and methods of identifying cancers responsive to a Siglec-9 ECD or Siglec-9 ECD fusion molecule, for example by determining the level of CD163 and/or Siglec-9, and in some embodiments also CD68 and/or sialic acid expression in tumor or immune cells.

### BACKGROUND

Sialic acid-binding Ig-like lectin-9 (Siglec-9) is a type 1, immunoglobulin-like, transmembrane protein expressed on immune and hematopoietic cells, including immature and mature myeloid cells, such as monocytes, macrophages, dendritic cells, neutrophils, and microglia, as well as lymphoid cells, such as natural killer cells and subsets of T cells (Crocker et al. (2007) Nat Rev Immunol. 7:255-266; O'Reilly and Paulson (2009) Trends in Pharm. Sci. 30:5:240-248; and Macauley et al. (2014) Nat. Rev. Imm. 14: 653-666). Siglec-9 is a member of the Siglec family of lectins that bind sialic acid residues of glycoproteins and glycolipids. Potential ligands for Siglec proteins are gangliosides, which are glycolipids comprising a ceramide linked to a sialylated glycan. Diversity in the Siglec ligands is generated by the addition of other neutral sugars and sialic acid in different linkages, either branched or terminal, and modification of sialic acid itself.

Fourteen Siglec proteins have been identified in humans and nine in mice that are comprised of 2-17 extracellular Ig domains including an amino-terminal V-set Ig-like (IgV) domain that contains the sialic acid binding site. The IgV domain contains two aromatic residues and one arginine in a motif that is highly conserved in all Siglecs (Crocker et al. (2007) Nat Rev Immunol. 7:255-266; McMillan and Crocker (2008) Carbohydr Res. 343:2050-2056; Von Gunten and Bochner (2008) Ann NY Acad Sci. 1143:61-82; May et al. (1998) Mol Cell. 1:719-728; Crocker et al. (1999) Biochem J. 341:355-361; and Crocker and Varki (2001) Trends Immunol. 2:337-342). The ligand binding sites have been mapped by crystal structures with and without ligand bound (Attrill et al., (2006) J. Biol. Chem. 281 32774-32783; Alphey et al. (2003) J. Biol. Chem. 278:5 3372-3377; Varki et al., Glycobiology, 16 pp. 1R-27R; and May et al. (1998) Mol. Cell 1:5:719-728). Because cell membranes are rich in sialic acids, ligand binding by Siglecs can occur in cis and in trans, which affects their functional properties. Each Siglec has a distinct preference for binding the diverse types of sialylated glycans that are found on the surface of mammalian cells (Crocker et al. (2007) Nat Rev Immunol. 7:255-266; and Crocker et al. (2007) Nat Rev Immunol. 7:255-266).

Most Siglec proteins, including Siglec-9, are inhibitory receptors that contain one or more immunoreceptor tyrosine-based inhibitory motif (ITIM) sequences in their cytoplasmic domains. The inhibitory Siglecs act as negative regulators of immune function (Crocker et al. (2007) Nat Rev Immunol. 7:255-266; McMillan and Crocker (2008) Carbohydr Res. 343:2050-2056; and Von Gunten and Bochner (2008) Ann NY Acad Sci. 1143:61-82). Other Siglecs are activating receptors that contain immunoreceptor tyrosine-based activating motif (ITAM) sequences in their cytoplasmic domains. Those Siglecs act as positive regulators of immune function (Macauley SM. et al., (2014) Nature Reviews Immunology 14, 653-666). See also Smith and Bertozzi, Nat Rev Discov 20:217-243 (2021); Rodriguez et al., Nat Rev Immunol 18:204-211 (2019) for further reviews of Siglec proteins.

The Siglec protein family plays a role in tumor pathogenesis. Many human tumors robustly upregulate sialic acid ligands that bind Siglec-9, which may enable immune evasion and cancer progression (Jandus et al. (2014) J. Clinic. Invest. 124:1810-1820). In contrast, tumors lacking sialic acid biosynthesis have reduced growth in mice (Stanczak et al. (2018) J Clin Invest. 128:4912-4923). Certain SNPs in Siglec-3, 7, 9 are associated with decreased risk of colorectal and lung cancer (Id.).

All references cited herein, including patent applications and publications, are hereby incorporated by reference in their entirety.

### SUMMARY

The present disclosure is generally directed to Siglec-9 extracellular domain (ECD) fusion proteins or Siglec-9 ECDs and methods of treating certain types of cancer using Siglec-9 ECDs or Siglec-9 ECD fusion molecules. For example, tumors increase the expression of sialic acid glycans and co-opt the immunosuppressive effects of Siglecs, driving tumor resident immune cells toward a cancer-permissive phenotype. Data provided in the Examples herein show that Siglelc-9 shows high expression relative to most other inhibitory Siglecs in samples from a variety of tumor types. The data further show that a variety of tumor types have resident immune cells (including MDSCs) that express high levels of CD163, which is a marker for an M2 macrophage phenotype. Accordingly, tumors with resident immune cells that are high in CD163 and/or Siglec-9, as well as in some cases CD68 and other inhibitory Siglecs, may be most likely to respond to treatment with a Siglec-9 ECD fusion protein. Furthermore, expression levels of CD163 and/or Siglec-9, as well as in some cases CD68 and sialic acid may be used to predict which patients would respond to a Siglec-9 ECD or a Siglec-9 ECD fusion molecule.

Accordingly, this disclosure includes many embodiments, including but not limited to, the following embodiments.

Embodiment 1 is a method of treating cancer in a subject in need thereof, comprising administering to the subject a Siglec-9 extracellular domain (ECD) fusion polypeptide, wherein an elevated expression level of CD163 and/or Siglec-9 has been detected in a tumor sample from the subject.

Embodiment 2 is a method of predicting a response to treatment with a Siglec-9 extracellular domain (ECD) fusion polypeptide in a subject having cancer, comprising determining an expression level of CD163 and/or Siglec-9 in a tumor sample from the subject, wherein an elevated expression level of CD163 and/or Siglec-9 predicts response to treatment with the Siglec-9 ECD fusion polypeptide.

Embodiment 3 is a method of selecting a subject with cancer for treatment with a Siglec-9 extracellular domain (ECD) fusion polypeptide, comprising selecting the subject for such treatment if a tumor sample from the subject has been determined to have an elevated expression level of CD163 and/or Siglec-9.

Embodiment 4 is the method of embodiment 2 or 3, further comprising administering a Siglec-9 ECD fusion polypeptide to the subject.

Embodiment 5 is a method of treating cancer in a subject in need thereof, comprising determining that a tumor sample from the subject has an elevated expression level of CD163 and/or Siglec-9, and administering a Siglec-9 extracellular domain (ECD) fusion polypeptide to the subject.

Embodiment 6 is the method of any one of embodiments 1-5, wherein the tumor sample from the subject has elevated expression levels of CD163 and Siglec-9.

Embodiment 7 is the method of any one of embodiments 1-6, wherein the tumor sample from the subject has an elevated expression level of CD68.

Embodiment 8 is the method of any one of embodiments 1-7, wherein the tumor sample from the subject has an elevated level of sialic acid.

Embodiment 9 is the method of any one of embodiments 1-8, wherein the tumor sample from the subject is obtained from a tumor biopsy.

Embodiment 10 is the method of any one of embodiments 1-9, wherein the expression level of CD163 and/or Siglec-9, and/or optionally CD68, and/or optionally the level of sialic acid, is detected by immunohistochemistry (IHC).

Embodiment 11 is the method of embodiment 10, wherein the expression level of CD163, Siglec-9, or CD68 or the level of sialic acid is determined to be elevated if IHC detects 20-100 stained cells per high power field, or greater than 100 stained cells per high power field, or if the IHC score on a scale of 0-3 is IHC 2 or IHC 3, for CD163, Siglec-9, CD68 or sialic acid.

Embodiment 12 is the method of embodiment 10 or 11, wherein the expression level of CD163, Siglec-9, or CD68 or the level of sialic acid is determined to be elevated if the IHC score is IHC 2 or IHC 3.

Embodiment 13 is the method of embodiment 10 or 11, wherein the expression levels of CD163 and Siglec-9 are determined to be elevated, and the IHC scores for CD163 and Siglec-9 are selected from: CD163 IHC 3 and Siglec-9 IHC 3; CD163 IHC 2 and Siglec-9 IHC 3; CD163 IHC 3 and Siglec-9 IHC 2; or CD163 IHC 2 and Siglec-9 IHC 2.

Embodiment 14 is the method of embodiment 10 or 11, wherein the expression levels of CD163, Siglec-9 and CD68 are determined to be elevated, and the IHC scores for CD163, Siglec-9, and CD68 are selected from: CD163 IHC 3, Siglec-9 IHC 3, and CD68 IHC 3; CD163 IHC 2, Siglec-9 IHC 3, and CD68 IHC 3: CD163 IHC 3. Siglec-9 IHC 2, and CD68 IHC 3; CD163 IHC 2, Siglec-9 IHC 2, and CD68 IHC 3; CD163 IHC 3, Siglec-9 IHC 3, and CD68 IHC 2; CD163 IHC 2. Siglec-9 IHC 3, and CD68 IHC 2: CD163 IHC 3, Siglec-9 IHC 2, and CD68 IHC 2; or CD163 IHC 2, Siglec-9 IHC 2, and CD68 IHC 2.

Embodiment 15 is the method of embodiment 10 or 11, wherein the expression levels of CD163 and Siglec-9 and the level of sialic acid are determined to be elevated, and the IHC scores for CD163, Siglec-9, and sialic acid are selected from: CD163 IHC 3, Siglec-9 IHC 3, and sialic acid IHC 3; CD163 IHC 2, Siglec-9 IHC 3, and sialic acid IHC 3; CD163 IHC 3, Siglec-9 IHC 2, and sialic acid IHC 3; CD163 IHC 2, Siglec-9 IHC 2, and sialic acid IHC 3; CD163 IHC 3, Siglec-9 IHC 3, and sialic acid IHC 2; CD163 IHC 2, Siglec-9 IHC 3, and sialic acid IHC 2; CD163 IHC 3, Siglec-9 IHC 2, and sialic acid IHC 2; or CD163 IHC 2, Siglec-9 IHC 2, and sialic acid IHC 2.

Embodiment 16 is the method of embodiment 10 or 11, wherein the expression levels of CD163, Siglec-9 and CD68 and the level of sialic acid are determined to be elevated, and the IHC scores for CD163, Siglec-9, CD68, and sialic acid are selected from: CD163 IHC 3, Siglec-9 IHC 3, CD68 IHC 3, and sialic acid IHC 3; CD163 IHC 2, Siglec-9 IHC 3, CD68 IHC 3, and sialic acid IHC 3; CD163 IHC 3, Siglec-9 IHC 2, CD68 IHC 3, and sialic acid IHC 3; CD163 IHC 2, Siglec-9 IHC 2, CD68 IHC 3, and sialic acid IHC 3; CD163 IHC 3, Siglec-9 IHC 3, CD68 IHC 2, and sialic acid IHC 3; CD163 IHC 2, Siglec-9 IHC 3, CD68 IHC 2, and sialic acid IHC 3; CD163 IHC 3, Siglec-9 IHC 2, CD68 IHC 2, and sialic acid IHC 3; CD163 IHC 2, Siglec-9 IHC 2, CD68 IHC 2, and sialic acid IHC 3; CD163 IHC 3, Siglec-9 IHC 3, CD68 IHC 3, and sialic acid IHC 2; CD163 IHC 2, Siglec-9 IHC 3, CD68 IHC 3, and sialic acid IHC 2; CD163 IHC 3, Siglec-9 IHC 2, CD68 IHC 3, and sialic acid IHC 2; CD163 IHC 2, Siglec-9 IHC 2, CD68 IHC 3, and sialic acid IHC 2: CD163 IHC 3, Siglec-9 IHC 3, CD68 IHC 2, and sialic acid IHC 2; CD163 IHC 2, Siglec-9 IHC 3, CD68 IHC 2, and sialic acid IHC 2; CD163 IHC 3, Siglec-9 IHC 2, CD68 IHC 2, or sialic acid IHC 2; CD163 IHC 2, Siglec-9 IHC 2, CD68 IHC 2, and sialic acid IHC 2.

Embodiment 17 is the method of any one of embodiments 10-16, wherein IHC for Siglec-9 is conducted with an anti-Siglec-9 antibody comprising an HVR-L1 comprising the amino acid sequence of SEQ ID NO: 240, an HVR-L2 comprising the amino acid sequence of SEQ ID NO: 241, an HVR-L3 comprising the amino acid sequence of SEQ ID NO: 242, an HVR-H1 comprising the amino acid sequence of SEQ ID NO: 243, an HVR-H2 comprising the amino acid sequence of SEQ ID NO: 244, and an HVR-H3 comprising the amino acid sequence of SEQ ID NO: 245.

Embodiment 18 is the method of any one of embodiments 10-17, wherein IHC for Siglec-9 is conducted with an anti-Siglec-9 antibody comprising a heavy chain variable domain comprising the amino acid sequence of SEQ ID NO: 247 and a light chain variable domain comprising the amino acid sequence of SEQ ID NO: 246, optionally wherein the antibody is a murine IgG2A antibody.

Embodiment 19 is the method of any one of embodiments 1-18, wherein the cancer is a solid tumor associated with a tumor microenvironment comprising myeloid cells.

Embodiment 20 is the method of any one of embodiments 1-19, wherein the cancer is selected from renal cell carcinoma, sarcoma, pancreatic cancer, glioblastoma, ovarian cancer, colorectal cancer, lung cancer, melanoma, head and neck cancer, breast cancer, and gastric cancer.

Embodiment 21 is the method of any one of embodiments 1-20, wherein the cancer is metastatic.

Embodiment 22 is the method of any one of embodiments 1-21, wherein the Siglec-9 ECD fusion polypeptide comprises a Siglec-9 IgV domain comprising an amino acid sequence selected from any one of SEQ ID NOs: 109-137 and 214-226.

Embodiment 23 is the method of embodiment 22, wherein the polypeptide comprises a Siglec-9 ECD comprising the Siglec-9 IgV domain, a C2 type 1 (C2T1) domain, and a C2 type 2 (C2T2) domain.

Embodiment 24 is the method of embodiment 22 or embodiment 23, wherein the polypeptide comprises an amino acid sequence selected from any one of SEQ ID NOs: 79-107 and 194-206.

Embodiment 25 is the method of any one of embodiments 22-24, wherein the polypeptide further comprises an Fc domain.

Embodiment 26 is the method of embodiment 25, wherein the Fc domain is located at the C-terminus of the polypeptide.

Embodiment 27 is the method of embodiment 25 or embodiment 26, wherein the Fc domain has a human IgG1 isotype.

Embodiment 28 is the method of embodiment 27, wherein the Fc domain has a human IgG1 isotype that has:
a) reduced binding to FcγRIII;
b) reduced antibody-dependent cellular cytotoxicity (ADCC) and/or reduced complement binding activity;
c) increased binding to FecγRIIa; or
d) any combination of a), b), and/or c).
   relative to the IgG1 polypeptide of SEQ ID No: 142.

Embodiment 29 is the method of embodiment 25 or 26, wherein the Fc domain comprises an amino acid sequence selected from any one of SEQ ID NOs: 142-144 and 234-239.

Embodiment 30 is the method of embodiment 29, wherein the Fc domain comprises the amino acid sequence of SEQ ID NO: 142 or 143.

Embodiment 31 is the method of embodiment 25 or 26, wherein the polypeptide comprises an amino acid sequence selected from any one of SEQ ID NOs: 11-39, 148-160, and 168-170.

Embodiment 32 is the method of embodiment 31, wherein the polypeptide comprises an amino acid sequence selected from any one of SEQ ID NOs: 49-77, 171-183, and 191-193.

Embodiment 33 is the method of any one of embodiments 1-21, wherein the Siglec-9 ECD fusion polypeptide comprising an amino acid sequence selected from any one of SEQ ID NOs: 49-77 and 171-193, lacking its signal peptide.

Embodiment 34 is the method of embodiment 25 or embodiment 26, wherein the Fc domain has a human IgG4 isotype.

Embodiment 35 is the method of embodiment 34, wherein the Fc domain comprises the amino acid sequence of SEQ ID NO: 145 or 146.

Embodiment 36 is the method of any one of embodiments 1-21, wherein the Siglec-9 ECD fusion polypeptide comprises the amino acid sequence of SEQ ID NO: 138.

Embodiment 37 is the method of embodiment 36, wherein the polypeptide further comprises an Fc domain.

Embodiment 38 is the method of embodiment 37, wherein the Fc domain is located at the C-terminus of the polypeptide.

Embodiment 39 is the method of embodiment 38, wherein the Fc domain has a human IgG1 isotype.

Embodiment 40 is the method of embodiment 39, comprising a linker sequence.

Embodiment 41 is the method of embodiment 37 or 38, wherein the Fc domain comprises an amino acid sequence selected from any one of SEQ ID NOs: 142-144.

Embodiment 42 is the method of embodiment 41, wherein the Fc domain comprises the amino acid sequence of SEQ ID NO: 142 or 143.

Embodiment 43 is the method of embodiment 39, wherein the polypeptide comprises the amino acid sequence of SEQ ID NO: 139.

Embodiment 44 is the method of embodiment 37, wherein the Fc domain has a human IgG4 isotype.

Embodiment 45 is the method of embodiment 44, wherein the Fc domain comprises the amino acid sequence of SEQ ID NO: 145 or 146.

Embodiment 46 is the method of any one of embodiments 1-21, wherein the Siglec-9 ECD fusion polypeptide comprises SEQ ID NO: 78 joined at its C-terminus to an Fc domain.

Embodiment 47 is the method of embodiment 46, wherein the Fc domain has a human IgG1 or IgG4 isotype.

Embodiment 48 is the method of embodiment 47, wherein the Fc domain has a human IgG1 isotype that has:
e) reduced binding to FcγRIII;
f) reduced antibody-dependent cellular cytotoxicity (ADCC) and/or reduced complement binding activity;
g) increased binding to FcγRIIa; or
h) any combination of a), b), and/or c),
   relative to the IgG1 polypeptide of SEQ ID No: 142.

Embodiment 49 is the method of embodiment 47 or 48, wherein the Fc domain has a human IgG1 isotype and comprises an amino acid sequence selected from any one of SEQ ID NOs: 142-144 and 234-239.

Embodiment 50 is the method of embodiment 47, wherein the Fc domain comprises the amino acid sequence of SEQ ID NO: 142.

Embodiment 51 is the method of embodiment 47, wherein the polypeptide comprises the amino acid sequence of SEQ ID NO: 10.

Embodiment 52 is the method of embodiment 47 or 48, wherein the Fc domain comprises the amino acid sequence of SEQ ID NO: 143.

Embodiment 53 is the method of embodiment 47, wherein the polypeptide comprises the amino acid sequence of SEQ ID NO: 227.

Embodiment 54 is the method of embodiment 47, wherein the Fc domain has a human IgG4 isotype and comprises the amino acid sequence of SEQ ID NO: 145 or 146.

Embodiment 55 is the method of embodiment 46, wherein the polypeptide comprises an amino acid sequence selected from any one of SEQ ID NOs: 45-48 and 228-233, lacking its associated signal peptide.

Embodiment 56 is the method of embodiment 55, wherein the polypeptide comprises an amino acid sequence selected from any one of SEQ ID NOs: 45-48 and 228-233.

Embodiment 57 is the method of embodiment 55, wherein the polypeptide comprises the amino acid sequence of SEQ ID NO: 45, lacking its associated signal peptide.

Embodiment 58 is the method of embodiment 57, wherein the polypeptide comprises the amino acid sequence of SEQ ID NO: 45.

Embodiment 59 is the method of embodiment 55, wherein the polypeptide comprises the amino acid sequence of SEQ ID NO: 48, lacking its associated signal peptide.

Embodiment 60 is the method of any one of embodiments 1-21, wherein the Siglec-9 ECD fusion polypeptide comprises an amino acid sequence selected from any one of SEQ ID NOs: 207-213 and an Fc domain located at the C-terminus of the polypeptide.

Embodiment 61 is the method of embodiment 60, wherein the Fc domain has a human IgG1 or IgG4 isotype.

Embodiment 62 is the method of embodiment 61, wherein the Fc domain has a human IgG1 isotype that has:
i) reduced binding to FcγRIII;
j) reduced antibody-dependent cellular cytotoxicity (ADCC) and/or reduced complement binding activity;
k) increased binding to FcγRIIa; or
l) any combination of a), b), and/or c),
   relative to the IgG1 polypeptide of SEQ ID No: 142.

Embodiment 63 is the method of embodiment 61 or 62, wherein the Fc domain has a human IgG1 isotype and comprises an amino acid sequence selected from any one of SEQ ID NOs: 142-144 and 234-239.

Embodiment 64 is the method of embodiment 60, comprising an amino acid sequence selected from any one of SEQ ID NOs: 161-167.

Embodiment 65 is the method of embodiment 61, comprising an amino acid sequence selected from any one of SEQ ID NOs: 184-190, lacking its associated signal peptide.

Embodiment 66 is the method of embodiment 65, comprising an amino acid sequence selected from any one of SEQ ID NOs: 184-190.

Embodiment 67 is the method of embodiment 61, wherein the Fc domain has a human IgG4 isotype and comprises the amino acid sequence of SEQ ID NO: 145 or 146.

Embodiment 68 is the method of any one of embodiments 1 or 4-67, further comprising administering an antagonist of PD-1 or PD-L1, optionally wherein the antagonist of PD-1 or PD-L1 is an antibody that binds to PD-1 or PD-L1, respectively.

Embodiment 69 is the method of any one of embodiments 1 or 4-68, further comprising administering a chemotherapeutic agent.

### BRIEF DESCRIPTION OF THE DRAWINGS

FIG. 1 shows a schematic depicting the synapse between a hypersialylated tumor cell and an innate immune cell, such as an MDSC, displaying exemplary immune inhibitory Siglecs, sialic acids (Siglec ligand), and macrophage markers, such as CD68 and CD163.

FIG. 2 shows a diagram of the rationale for identification and optimization of potential predictive biomarkers. IHC, immunohistochemistry: RNA, ribonucleic acid.

FIG. 3 shows relative expression of inhibitory Siglecs across tumor types. FPKM, fragments per kilobase million. NHSCC, head and neck squamous cell carcinoma.

FIG. 4 shows representative immunohistochemistry (IHC) staining for sialic acid. CD163. CD68, and Siglec-9 in the same gastric cancer sample. 10841 FIG. 5 shows representative IHC staining for sialic acid. CD163. CD68, and Siglec-9 in the same lung squamous cell carcinoma sample.

FIG. 6 shows representative IHC staining for sialic acid. CD163. CD68, and Siglec-9 in the same ovarian cancer sample.

FIG. 7 shows IHC profiling by IHC staining of immune cells associated with tumors and of tumor cells, in each case from a variety of tumor types. CLL, chronic lymphocytic leukemia: DLBCL, diffuse large B-cell lymphoma; ER+, estrogen receptor positive; GIST, gastrointestinal stromal tumor; HER2+, human epidermal growth factor receptor 2 positive; HNSCC, head and neck squamous cell carcinoma; SCC, squamous cell carcinoma; TNBC, triple negative breast cancer. IHC 0, <10 stained cells per high power field: IHC 1, 10-20 stained cells per high power field: IHC 2, <20-100 stained cells per high power field; IHC 3, >100 stained cells per high power field.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DETAILED DESCRIPTION

Provided herein are methods of treating certain types of cancer using Siglec-9 ECDs or Siglec-9 ECD fusion molecules, including cancers expressing high levels of CD163 and/or Siglec-9 in tumor or immune cells, and methods of identifying cancers responsive to a Siglec-9 ECD or Siglec-9 ECD fusion molecule, for example by determining the level of CD163 and/or Siglec-9, and in some embodiments also CD68 and/or sialic acid expression in tumor or immune cells.

### Definitions

The terms “Siglec-9 extracellular domain” and “Siglec-9 ECD” refer to an extracellular domain polypeptide of Siglec-9 or a fragment thereof that binds sialic acid on the surface of cells. The terms include natural and engineered variants thereof. In some embodiments, a Siglec-9 ECD comprises the IgV domain of Siglec-9. In some embodiments, a Siglec-9 ECD comprises the IgV domain and the C2 type 1 (C2T1) domain and the C2 type 2 (C2T2) domain of Siglec-9. Nonlimiting exemplary Siglec-9 ECDs are shown in SEQ ID NOs: 78-138.

The term “Siglec-9 ECD fusion molecule” or “Siglec-9 ECD fusion polypeptide” interchangeably refer to a molecule comprising a Siglec-9 ECD and a covalently-attached fusion partner, such as an Fc domain, albumin, or polyethylene glycol (PEG). In some embodiments, the fusion partner is attached to the C-terminus of the Siglec-9 ECD. A Siglec-9 ECD fusion molecule in which the fusion partner is an Fc domain may also be referred to herein as a “Siglec-9 ECD-Fc fusion molecule,” “Siglec-9 ECD-Fc fusion polypeptide,” a “Siglec-9 ECD-Fc,” or a “Siglec-9-Fc.” Nonlimiting exemplary Siglec-9 ECD-Fc fusion molecules are shown in the amino acid sequences of SEQ ID NOs: 10-77 and 139, including those sequences with or without their associated signal peptides.

The term “specific binding” or “specifically binds” or is “specific for” a target moiety means binding that is measurably different from a non-specific interaction. Specific binding can be measured, for example, by determining binding of a test molecule for the target moiety compared to binding of the test molecule for a control moiety. The test molecule specifically binds the target moiety if the binding affinity for the target moiety is at least 2-fold, or at least 3-fold, or at least 5-fold, or at least 10-fold stronger than the binding affinity for the control moiety. For the avoidance of doubt, specific binding does not require that a test molecule does not bind any other moieties.

An “amino acid modification” at a specified position, e.g., of a Siglec-9 ECD of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

The term “Fc region” herein is used to mean a C-terminal region of an immunoglobulin heavy chain, including native sequence Fc regions and variant Fc regions. Although the boundaries of the Fc region of an immunoglobulin heavy chain might vary, the human IgG heavy chain Fc region is generally defined as including a polypeptide from an amino acid residue at position Cys226 or from Pro230, to the carboxyl-terminus thereof. The C-terminal lysine (residue 447 according to the EU numbering system) of the Fc region may be removed, for example, during production or purification of an Fc region-containing polypeptide, or by recombinantly engineering the nucleic acid encoding the Fc region-containing polypeptide. Suitable native-sequence Fc regions for use in the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); a native sequence human IgG2 Fe region; a native sequence human IgG3 Fc region; and a native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region, e.g. from about one to about ten amino acid substitutions, and preferably from about one to about five amino acid substitutions in a native sequence Fe region. The variant Fc region herein will preferably possess at least about 80% homology with a native sequence Fc region, and most preferably at least about 90% homology therewith, more preferably at least about 95% homology therewith.

“Fe receptor” or “FcR” describes a receptor that binds to the Fc region. The preferred FcR is a native sequence human FcR. Moreover, a preferred FcR is one which binds an IgG Fc region (a gamma receptor) and includes receptors of the FeγRI, FeγRII, and FcγRIII subclasses, including allelic variants and alternatively spliced forms of these receptors. FeγRII receptors include FeγRIIA (an “activating receptor”) and FeγRIIB (an “inhibiting receptor”), which have similar amino acid sequences that differ primarily in the cytoplasmic domains thereof. Activating receptor FeγRIIA contains an immunoreceptor tyrosine-based activation motif (“ITAM”) in its cytoplasmic domain. Inhibiting receptor FeγRIIB contains an immunoreceptor tyrosine-based inhibition motif (“ITIM”) in its cytoplasmic domain. Other FcRs are encompassed by the term “FcR” herein. FcRs can also increase the serum half-life of molecules that comprise Fc regions.

Binding to FcR in vivo and serum half-life of human FcR high-affinity binding polypeptides can be assayed, e.g., in transgenic mice or transfected human cell lines expressing human FcR, or in primates to which the polypeptides having a variant Fe region are administered. WO 2004/42072 (Presta) describes Fe region variants with improved or diminished binding to FcRs. See also, e.g., Shields et al., J. Biol. Chem. 9(2):6591-6604 (2001).

As used herein, “percent (%) amino acid sequence identity” and “homology” with respect to a reference polypeptide sequence refers to the percentage of amino acid residues in a query sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST. BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

An “isolated” nucleic acid molecule encoding a polypeptide, such as a polypeptide comprising a Siglec-9 ECD of the present disclosure, is a nucleic acid molecule that is identified and separated from at least one contaminant molecule with which it is ordinarily associated in the environment in which it was produced. Preferably, the isolated nucleic acid is free of association with most or substantially all components associated with the production environment. The isolated nucleic acid molecules encoding the polypeptides herein are distinguished from nucleic acids existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can contain or contains a vector(s) or other exogenous nucleic acid, e.g., that incorporates a polynucleotide insert(s). In some embodiments, the vector or other exogenous nucleic acid is incorporated into the genome of the host cell. Host cells include progeny of a single host cell, and the progeny may not necessarily be completely identical (in morphology or in genomic DNA complement) to the original parent cell due to natural, accidental, or deliberate mutation. A host cell includes cells comprising (e.g., transfected with) a polynucleotide(s) of this invention.

As used herein, “expression level” and “level” refer to a measurement that is made using any analytical method for detecting a nucleic acid, a protein, or other molecule in a biological sample and that indicates the presence, absence, absolute amount or concentration, relative amount or concentration, titer, ratio of measured levels, or the like, of, for, or corresponding to a protein, such as CD163, CD68, or Siglec-9, or to another molecule or moiety, such as sialic acid, in the biological sample. The exact nature of the “level” depends on the specific design and components of the particular analytical method employed for detection. In some embodiments, an expression level of a protein or level of another molecule or moiety is determined by immunohistochemistry (IHC).

As used herein, “elevated expression level” or “elevated level” refers to a measurement that shows an increased concentration or amount of a protein, molecule or moiety in a biological sample, such as a tumor sample, relative to a reference biological sample or falling within a specified range predetermined to indicate an increased concentration or amount. In some embodiments, the measurement may be performed by immunohistochemistry (IHC). As described in certain exemplary embodiments herein, an elevated expression level or elevated level as determined by IHC may be characterized by a score of IHC 2 or IHC 3.

The term “detecting” as used herein encompasses quantitative or qualitative detection.

A “tumor sample” herein refers to a sample comprising or expected to comprise tumor cells that is obtained from a patient. A tumor sample may further comprise immune cells, e.g., innate immune cells, such as myeloid-derived suppressor cells (MDSCs) or macrophages, and/or adaptive immune cells, such as T-cells. By way of example, a tumor sample can be a biopsy obtained from a solid tumor, or a tumor sample can be a blood or plasma sample in the case of a blood-based cancer. In some cases, the tumor sample can be fixed onto a slide for analysis of levels of certain proteins or other molecules in cells from the sample.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins: hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA: sugar alcohols such as mannitol or sorbitol: salt-forming counterions such as sodium; and/or nonionic surfactants such as TWEEN™, polyethylene glycol (PEG), and PLURONICS™.

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the terms “treat,” “treatment,” “treating,” and the like refer to clinical intervention designed to alter the natural course of a clinical pathology in the individual being treated. Desirable effects of treatment include decreasing the rate of progression, ameliorating or palliating the pathological state, remission or improved prognosis, and/or alleviating or lessening the symptoms of a particular disease, disorder, or condition. An individual is successfully “treated”, for example, if one or more symptoms associated with a particular disease, disorder, or condition are mitigated or eliminated. In certain embodiments, a patient is successfully “treated” for cancer according to the methods of the present invention if the patient shows one or more of the following: a reduction in the number of or complete absence of cancer cells; a reduction in the tumor size: inhibition of or an absence of cancer cell infiltration into peripheral organs including, for example, the spread of cancer into soft tissue and bone; inhibition of or an absence of tumor metastasis; inhibition of or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), overall survival (OS), complete response (CR), partial response (PR), or stable disease (SD): a decrease in progressive disease (PD); reduced time to progression (TTP); or any combination thereof.

The terms “administer,” “administering,” “administration,” and the like refer to methods that may be used to enable delivery of a therapeutic agent such as a Siglec-9 ECD (e.g., a Siglec-9 ECD-Fc fusion molecule). Administration techniques that can be employed with the agents and methods described herein are found in e.g., Goodman and Gilman, The Pharmacological Basis of Therapeutics, current edition, Pergamon; and Remington's, Pharmaceutical Sciences, current edition. Mack Publishing Co., Easton, Pa.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” or “subject” or “patient” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the “individual” or “subject” or “patient” is human.

The terms “cancer” and “cancerous” refer to or describe the physiological condition in mammals in which a population of cells are characterized by unregulated cell growth. The cancer may be a primary tumor or may be advanced or metastatic cancer. A “refractory” cancer is one that progresses even though an anti-tumor treatment has been administered to the cancer patient. A “recurrent” cancer, or a cancer that has “recurred,” is one that has regrown, either at the initial site or at a distant site, after a response to initial therapy. A “relapsed” patient is one who has signs or symptoms of cancer after remission. Optionally, the patient has relapsed after adjuvant or neoadjuvant therapy.

As used herein, administration of an agent or composition “in conjunction” or “in combination” with another agent or composition includes simultaneous administration and/or administration at different times. Administration in conjunction also encompasses administration as a co-formulation or administration as separate compositions, including at different dosing frequencies or intervals, and using the same route of administration or different routes of administration. In some embodiments, administration in conjunction means administration as a part of the same treatment regimen. In some embodiments, administration of an agent in combination with another agent results in “synergy” or a “synergistic effect,” i.e., the effect achieved when the agents are used together is greater than the sum of the effects that result from using the agents separately. In some embodiments, administration of an agent in combination with another agent results in an “additive” effect, i.e., the effect achieved when the agents are used together is equal to the sum of the effects that result from using the agents separately.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise.

It is understood that aspect and embodiments of the present disclosure described herein include “comprising,” “consisting,” and “consisting essentially of” aspects and embodiments.

### Uses of a Siglec-9 Extracellular Domain (ECD) or Siglec-9 ECD Fusion Molecule in Cancer Treatments

As disclosed herein, Siglec-9 ECD, e.g., Siglec-9 ECD-Fc fusion molecules, of the present disclosure may be used as a therapeutic agent, e.g., for treating cancer. A therapeutic regimen may carried out by identifying a subject, e.g., a human patient suffering from a disease or disorder that would benefit from treatment with a Siglec-9 ECD or Siglec-9 ECD fusion molecule, such as cancer.

In some embodiments, the Siglec-9 ECD or Siglec-9 ECD fusion molecule may be used for treating cancer. In some embodiments, the cancer may have an elevated expression level of one or more molecules or proteins. In some embodiments, the expression level of one or more molecules or proteins may be measured in a tumor sample from the subject. In some embodiments, the expression level of CD163 and/or Siglec-9 may be detected in a tumor sample from the subject. In certain embodiments, the Siglec-9 ECD or Siglec-9 ECD fusion molecules may be used for treating cancer in a subject in which an elevated expression level of CD163 and/or Siglec-9 has been detected in a tumor sample from the subject.

In certain embodiments, methods of predicting a response to treatment of cancer with a Siglec-9 ECD or Siglec-9 ECD fusion molecule in a subject having cancer are disclosed. The methods in some cases comprise determining an expression level of CD163 and/or Siglec-9 in a tumor sample from the subject, wherein an elevated expression level of CD163 and/or Siglec-9 predicts responsiveness to treatment with a Siglec-9 ECD or Siglec-9 ECD fusion molecule. In some cases, the method may further comprise administering a Siglec-9 ECD or Siglec-9 ECD fusion molecule to a subject determined to have cancer with an elevated expression level of CD163 and/or Siglec-9.

In certain embodiments, methods of selecting a subject with cancer for treatment with a Siglec-9 ECD or Siglec-9 ECD fusion molecule are disclosed. The method comprises identifying a subject with cancer with an elevated expression level of CD163 and/or Siglec-9 in a tumor sample. The method may in some cases further comprise administering a Siglec-9 ECD or Siglec-9 ECD fusion molecule to a subject determined to have cancer with an elevated expression level of CD163 and/or Siglec-9.

In certain embodiments, methods of treating cancer in a subject in need thereof are provided, comprising determining that an elevated expression level of CD163 and/or Siglec-9 that has been detected in a tumor sample from the subject, and administering to the subject a Siglec-9 ECD or Siglec-9 ECD fusion molecule

In some embodiments, the expression levels of both CD163 and Siglec-9 are detected in the tumor sample. In some cases, the CD163 and/or Siglec-9 expression levels are detected in immune cells from the tumor sample. In some embodiments, the expression level of CD68 is also detected in the tumor sample, such as in immune cells from the tumor sample. In some embodiments, expression levels of CD68 may be detected in the tumor sample and tumor samples having elevated levels of CD68 identified. In some embodiments, the level of sialic acid is detected in the tumor sample. In some embodiments, levels of sialic acid may be detected in the tumor sample and tumor samples having elevated levels of sialic acid identified. Thus, in some cases, CD163 and/or Siglec-9 as well as one or both of CD68 and sialic acid levels are detected, and tumor samples having elevated expression levels of one or more of CD163, and/or Siglec-9, and/or CD68 identified. In certain embodiments, the level of CD163. Siglec-9, CD68, and/or sialic acid is determined by immunohistochemistry (IHC).

In certain embodiments, the tumor sample is obtained from a tumor biopsy. For example, in some embodiments, the tumor sample is a paraffin-embedded fixed biopsy sample. In some embodiments, the biopsy sample is fresh or frozen.

Exemplary methods of determining expression levels by IHC, which may be used in these methods, are described below.

In certain embodiments, the cancer to be treated is a solid tumor. The solid tumor may be associated with a tumor microenvironment comprising myeloid cells, e.g., macrophages, monocytes, microglia (in the CNS), dendritic cells, neutrophils, and/or granulocytes. In certain embodiments, the tumor microenviromnent comprises macrophages and monocytes. In certain embodiments, myeloid cells create an immunosuppressive tumor microenvironment in which a tumor can evade the immune system. Treatment with a Siglec-9 ECD fusion molecule herein may alleviate this suppression by activating mycloid cells and promoting an anti-tumor immune response.

In certain embodiments, a cancer to be treated by the methods of the present disclosure includes, without limitation, squamous cell carcinoma (e.g., epithelial squamous cell carcinoma), lung cancer, small-cell lung cancer, non-small cell lung cancer (NSCLC), squamous non-small cell lung cancer, adenocarcminoma of the lung, squamous carcinoma of the lung, non-squamous NSCLC, mesothelioma, glioma, cancer of the peritoneum, hepatocellular cancer, gastric cancer or stomach cancer including gastrointestinal cancer and gastrointestinal stromal cancer, renal cancer (e.g. clear cell carcinoma), ovarian cancer, liver cancer, colon cancer, colorectal cancer, small bowel cancer, urothelial cancer, endometrial cancer, hepatic carcinoma, kidney cancer (e.g., renal cell carcinoma (RCC)), prostate cancer (e.g. hormone refractory prostate adenocarcinoma), gallbladder cancer, thyroid cancer, neuroblastoma, sarcoma, pancreatic cancer, brain cancer (e.g., astrocytoma such as glioblastoma (glioblastoma multiforme)), cervical cancer, bladder cancer, hepatoma, breast cancer (e.g., triple negative breast cancer (TNBC). HER2+, ER+), and head and neck cancer (squamous cell carcinoma of the head and neck), melanoma (e.g., metastatic malignant melanoma, such as cutaneous or intraocular malignant melanoma), bone cancer, skin cancer, uterine cancer, anal cancer, testicular cancer, carcinoma of the fallopian tubes, vulval cancer, cholangiocarcinoma, mantle cell, and esophageal cancer. In certain embodiments, the cancer is selected from kidney cancer (e.g., renal cell carcinoma), sarcoma, pancreatic cancer, glioblastoma, ovarian cancer, colorectal cancer, lung cancer, melanoma, bladder cancer, head and neck cancer, breast cancer and uterine cancer. In some embodiments, the cancer is squamous cell lung cancer, colorectal cancer, ovarian cancer, or kidney cancer (such as renal cell carcinoma or clear cell renal cell carcinoma).

In certain embodiments, a cancer includes, without limitation, a hematopoietic cancer, such as a leukemia, lymphoma, or myeloma.

In some embodiments, the cancer may be an early stage cancer or a late stage cancer. In some embodiments, the cancer may be a primary tumor. In some embodiments, the cancer may be a metastatic tumor at a second site derived from any of the above types of cancer.

In some embodiments, a Siglec-9 ECD or Siglec-9 ECD fusion molecule may be administered in combination with an additional therapeutic agent that is used to treat the disease or pathology provided herein. The terms “in combination” and “in conjunction” are used interchangeably in the present disclosure. The additional therapeutic agent being administered in combination with the Siglec-9 ECD fusion molecule may be administered before, after, or concurrently with the Siglec-9 ECD fusion molecule.

In some embodiments, the cancer is refractory to checkpoint inhibitor therapy. In certain embodiments, the individual to be treated has a cancer that is refractory to therapy with a PD-1 or PD-L1 antagonist, e.g., a PD-1 or PD-L1 antibody, such as those provided below.

In some embodiments, the cancer to be treated has recurred after checkpoint inhibitor therapy. In certain embodiments, the individual to be treated has a cancer that has recurred after therapy with a PD-1 or PD-L1 antagonist, e.g., a PD-1 or PD-L1 antibody, such as those provided below.

In some embodiments, a Siglec-9 ECD, e.g., a Siglec-9 ECD-Fc fusion molecule, may be administered in conjunction with an antagonist of an inhibitory immune checkpoint molecule. In some embodiments, the inhibitory checkpoint molecule is PD-1 (programmed cell death protein-1) or its ligand PD-L1 (programmed death ligand-1). In some embodiments, an antagonist of PD-1 is an antibody to PD-1. PD-1 antibodies include, for example, OPDIVO (nivolumab), KEYTRUDA (pembrolizumab), MEDI-0680 (AMP-514; WO2012/145493), camrelizumab (SHR-1210), tislelizumab (BGB-A317), or spartalizumab (NPVPDR001, NVS240118, PDR00). A recombinant protein composed of the extracellular domain of PD-L2 (B7-DC) used to the Fc portion of IgG1, called AMP-224, can also be used to antagonize the PD-1 receptor. In some embodiments, an antagonist of PD-L1 is an antibody to PD-L1. PD-L1 antibodies include, for example. TECENTRIQ (atezolizumab), durvalumab (MEDI4736), BMS-936559 (WO2007/005874). MSB0010718C (WO2013/79174) or rHigM12B7. In some embodiments, a Siglec-9 ECD or Siglec-9 ECD fusion molecule of the present invention is administered in combination with radiation therapy and/or a chemotherapeutic agent.

### Administration

A Siglec-9 ECD or Siglec-9 ECD-Fc fusion molecule can be administered by any suitable means, including parenteral, intrapulmonary, intranasal, intratumoral, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous administration. In some embodiments, the administration is subcutaneous. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of disease, the appropriate dosage of a Siglec-9 ECD fusion molecule of the invention, such as a Siglec-9 ECD-Fc fusion molecule, when used alone or in combination with one or more other additional therapeutic agents, will depend on the type of disease to be treated, the type of fusion molecule, the severity and course of the disease, whether the fusion molecule is administered for preventive or therapeutic purposes, previous therapy, the patient's clinical history and response to the fusion molecule, and the discretion of the attending physician. The fusion molecule is suitably administered to the patient at one time or over a series of treatments.

### Methods of Determining CD163, Siglec-9, CD68, and Sialic Acid Levels

In some embodiments, the detection of expression levels may be quantitative or qualitative detection, e.g., determining CD163, Siglec-9, CD68, and/or sialic acid levels. Such detection in tumor samples may occur with any method known in the art, including immunofluorescence microscopy, immunocytochemistry, immunohistochemistry, ELISA, FACS analysis, immunoprecipitation, or micro-positron emission tomography. In certain embodiments, the detection is by immunohistochemistry (IHC).

In some embodiments, an expression level determined by IHC may be categorized by an IHC score. In some embodiments, the IHC score comprises a 4-point scale. In some such cases, the score is either 0, 1, 2, or 3, where IHC 0 comprises less than 10 stained cells per high power field, IHC 1 comprises 10-20 stained cells per high power field, IHC 2 comprises 20-100 stained cells per high power field, and IHC 3 comprises greater than 100 stained cells per high power field. In some embodiments, an elevated expression level comprises an IHC score of either 3 or 2 for each molecule or protein measured. In some embodiments, a “high power filed” is a tumor sample section, such as plated on a slide, that is measured at ×400 magnification with conventional light microscopy.

In some embodiments. CD163 and/or Siglec-9, and optionally further CD68 may be detected individually and an elevated expression level determined. Sialic acid may be detected individually and an elevated level determined. In some embodiments, CD163 and Siglec-9 are detected and an elevated expression level determined. In some embodiments, elevated expression levels of CD163, Siglec-9, CD68, and/or sialic acid levels may be detected according to the IHC scores shown in Table A.

TABLE A
Elevated IHC Scores

| | | CD68 | Sialic acid |
|---|---|---|---|
| CD163 | Siglec-9 | (optional) | (optional) |
| 3 | 3 | 3 | 3 |
| 2 | 3 | 3 | 3 |
| 3 | 2 | 3 | 3 |
| 2 | 2 | 3 | 3 |
| 3 | 3 | 2 | 3 |
| 2 | 3 | 2 | 3 |
| 3 | 2 | 2 | 3 |
| 2 | 2 | 2 | 3 |
| 3 | 3 | 3 | 2 |
| 2 | 3 | 3 | 2 |
| 3 | 2 | 3 | 2 |
| 2 | 2 | 3 | 2 |
| 3 | 3 | 2 | 2 |
| 2 | 3 | 2 | 2 |
| 3 | 2 | 2 | 2 |
| 2 | 2 | 2 | 2 |

In some embodiments, detection of CD163, Siglec-9. CD68, and/or sialic acid levels may be conducted with a commercially available antibody. In some embodiments, detection of Siglec-9 may be conducted with an anti-Siglec-9 antibody such as antibody 2D4, described in WO 2017/075432. In some embodiments, the anti-Siglec-9 antibody comprises an HVR-L1 comprising the amino acid sequence of SEQ ID NO: 240, an HVR-L2 comprising the amino acid sequence of SEQ ID NO: 241, an HVR-L3 comprising the amino acid sequence of SEQ ID NO: 242, an HVR-H1 comprising the amino acid sequence of SEQ ID NO: 243, an HVR-H2 comprising the amino acid sequence of SEQ ID NO: 244, and an HVR-H3 comprising the amino acid sequence of SEQ ID NO: 245. In some embodiments, the anti-Siglec-9 antibody comprises a heavy chain variable domain comprising the amino acid sequence of SEQ ID NO: 247 and a light chain variable domain comprising the amino acid sequence of SEQ ID NO: 246. In some embodiments, the anti-Siglec-9 antibody is a murine IgG1, IgG2A, or IgG2B antibody. In some embodiments, the anti-Siglec-9 antibody is a murine IgG2A antibody, and thus, comprises a murine IgG2A constant region. In some cases, sialic acid is detected using a Siglec-9 FCD-murine IgG1 Fc fusion polypeptide as IHC detection reagent. In some cases, CD163 is detected by IHC using a rabbit anti-human monoclonal antibody D6U1J (Cell Signaling Cat. No. 93498). In some cases, CD68 is detected by IHC using a rabbit anti-human CD68 monoclonal antibody (Cell Signaling, Cat #76437, Lot #1).

In some cases, using IHC, contrasting staining may be used to distinguish and co-localize the different proteins or markers such as CD163, Siglec-9, CD68, and sialic acid in a tumor sample. For example, different color fluorescent stains may be used for detection of different proteins within the sample. In some cases, and IHC score may then be determined by counting the apparent number of cells stained by the stain for the particular protein or molecule to be detected.

In certain embodiments, the detection of one or more of CD163, Siglec-9, CD68, or sialic acid may occur within the subject, for example with 18F and subsequently detected utilizing micro-positron emission tomography analysis. CD163, Siglec-9, CD68, expression levels or sialic acid levels may also be quantified in a subject, such as in a tumor in the subject, by non-invasive techniques such as positron emission tomography (PET), X-ray computed tomography, single-photon emission computed tomography (SPECT), computed tomography (CT), and computed axial tomography (CAT).

### Exemplary Siglec-9 ECDs and Siglec-9 ECD Fusion Molecules for Use in Methods Herein

In some embodiments, a Siglec-9 ECD or Siglec-9 ECD fusion molecule for use in methods herein may incorporate any of the features, singly or in combination, as described herein. Exemplary Siglec-9 ECDs and Siglec-9 ECD fusion molecule are described in US 2021-0284710 A1, which is incorporated by reference herein.

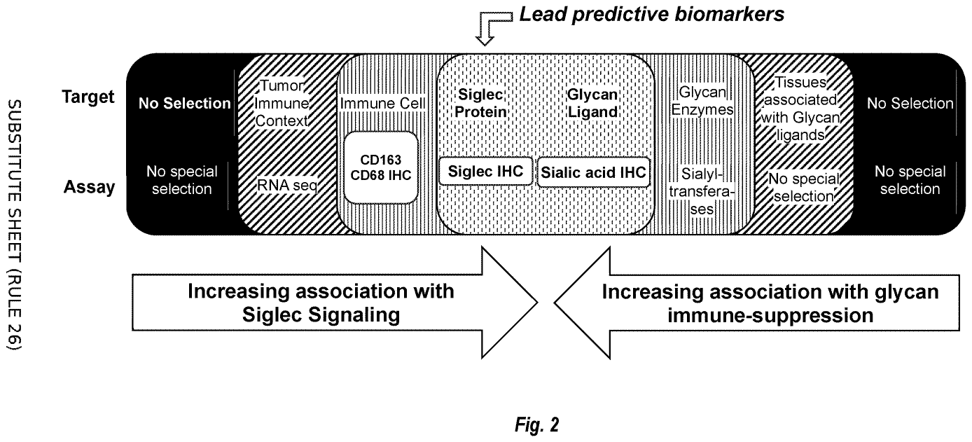

Examples are polypeptides comprising a Siglec-9 IgV domain. In certain embodiments, the Siglec-9 IgV domain comprises amino acids 20-140 of human Siglec-9 of SEQ ID NO: 1. See FIG. 2. As shown in Example 2 herein, the IgV domain of Siglec-9 is sufficient for binding to sialic acid on the surface of cells. In some embodiments, polypeptides are provided that comprise a Siglec-9 extracellular domain (ECD) comprising the IgV domain, the C2 type 1 (C2T1) domain, and the C2 type 2 (C2T2) domain. The Siglec-9 C2T1 domain comprises amino acids 146-229 of human Siglec-9 of SEQ ID NO: 1, and the Siglec-9 C2T2 domain comprises amino acids 236-336 of human Siglec-9 of SEQ ID NO: 1. In some embodiments, a Siglec-9 ECD comprises amino acids 20-336 of SEQ ID NO: 1, optionally with one or more amino acid modifications. In some embodiments, a Siglec-9 ECD comprises amino acids 20-336 of SEQ ID NO: 1, optionally with one or more amino acid modifications, and optionally with one to five amino acid deletions or additions on the N-terminus and/or C-terminus. In some embodiments, the Siglec-9 ECD may comprise the IgV, C2T1 and C2T2 domains, but may lack, for example, the last one, two, three, four, five, six, seven, eight, nine, ten, eleven or twelve C-terminal (membrane proximal) amino acids of the ECD. The twelve C-terminal (membrane proximal) amino acids of the ECD are shown in SEQ ID NO: 147. An example is SEQ ID NO: 78, for instance, which comprises the IgV, C2T1 and C2T2 domains and which lacks the C-terminal membrane proximal region.

In some embodiments, a polypeptide comprises a Siglec-9 IgV domain comprising one or more amino acid substitutions that improve stability of the polypeptide, improve the binding affinity for sialic acid, improve the function of the polypeptide, improve the pharmacokinetic properties of the polypeptide (e.g., half-life, Cmax, or AUC), or any combination of the foregoing. In some embodiments, a polypeptide comprises a Siglec-9 IgV domain having an amino acid sequence selected from any one of SEQ ID NOs: 108-137 and 214-226. In some embodiments, a polypeptide comprises a Siglec-9 IgV domain having an amino acid sequence selected from any one of SEQ ID NOs: 109-137 and 214-226. In some embodiments, a polypeptide comprises a Siglec-9 IgV domain having an amino acid sequence selected from any one of SEQ ID NOs: 108-137 and 214-226, optionally with one to five amino acid deletions or additions on the N-terminus and/or C-terminus. In some embodiments, a polypeptide comprises a Siglec-9 IgV domain having an amino acid sequence selected from any one of SEQ ID NOs: 109-137 and 214-226, optionally with one to five amino acid deletions or additions on the N-terminus and/or C-terminus. In some embodiments, a polypeptide comprises a Siglec-9 ECD with one or more substitutions C-terminal to the IgV domain. For example, in some embodiments the polypeptide comprises a Siglec-9 ECD of any one of SEQ ID Nos: 207-213. The sequence table below depicts the sequences corresponding to SEQ ID Nos listed herein. In many cases, locations of amino acid substitutions are shown in the table, such as by underlining, or by bolding and underlining, mutated residues.

In some embodiments, a polypeptide comprises a Siglec-9 ECD comprising one or more amino acid substitutions that improve stability of the polypeptide, improve the binding affinity for sialic acid, improve the function of the polypeptide, improve the pharmacokinetic properties of the polypeptide, or any combination of the foregoing. In some embodiments, a polypeptide comprises a Siglec-9 ECD having an amino acid sequence selected from any one of SEQ ID NOs: 78-107, 138, and 194-206. In some embodiments, a polypeptide comprises a Siglec-9 ECD having an amino acid sequence selected from any one of SEQ ID NOs: 78-107, 138, 194-206, optionally with one to five amino acid deletions or additions on the N-terminus and/or C-terminus.

In any of the embodiments provided herein, a polypeptide may further comprise a fusion partner. Nonlimiting exemplary fusion partners include Fe domains, albumin, and polyethylene glycol (PEG). In some embodiments, the fusion partner is covalently linked to the C-terminus of a Siglec-9 ECD. In some aspects, the fusion partner comprises an Fc domain. In some embodiments, a polypeptide comprising a Siglec-9 ECD and an Fc domain is provided herein, wherein the Fc domain is optionally fused to the C-terminus of the Siglec-9 ECD with or without an intervening linker sequence. A “linker sequence” as used herein refers to a polypeptide sequence not found in a native Siglec-9 ECD or its fusion partner (e.g., an Fc domain), wherein such polypeptide sequence is disposed between the Siglec-9 ECD and its fusion partner. In some embodiments, a linker sequence may be between about 4 and 25 amino acids. In some embodiments, the Fc domain is fused to the C-terminus without a linker sequence. In various embodiments, a polypeptide comprises a Siglec-9 ECD and an IgG1 Fc domain, e.g., the IgG1 Fc domain of SEQ ID NO: 142. In some embodiments, a polypeptide comprising a Siglec-9 ECD comprises an IgG1 Fc domain comprising NSLF substitutions, e.g., SEQ ID NO: 143. In some embodiments, a polypeptide comprising a Siglec-9 ECD comprises an IgG1 Fc domain comprising a K322A substitution, e.g., SEQ ID NO: 144. In some embodiments, a polypeptide comprising a Siglec-9 ECD comprises an IgG4 Fc domain or an IgG4 Fc domain comprising a S228P substitution, e.g., as shown in SEQ ID NOs: 145 or 146, respectively.

In some embodiments, a Siglec-9 ECD fusion molecule comprises an amino acid sequence selected from any one of SEQ ID NOs: 10-39, 148-160, and 168-170. In some embodiments, a Siglec-9 ECD fusion molecule comprises an amino acid sequence selected from any one of SEQ ID NOs: 40-77, 171-183, and 191-193, optionally lacking the signal sequence.

In some embodiments, a Siglec-9 ECD or a Siglec-9 ECD IgV domain of a Siglec-9 ECD fusion molecule comprises an amino acid sequence selected from any one of SEQ ID Nos: 109-137 and 214-226. In some cases, the Siglec-9 ECD comprises the IgV, C2T1, and C2T2 domains. In some embodiments, the Siglec-9 ECD lacks the membrane proximal region sequence of SEQ ID NO: 147 (MPR). In some embodiments, the Siglec-9 ECD comprises the IgV, C2T1, and C2T2 domains and lacks the MPR. In some embodiments, a Siglec-9 ECD comprises an amino acid sequence selected from any one of SEQ ID Nos: 79-107 and 194-206. In some embodiments, a Siglec-9 ECD comprises an amino acid sequence selected from any one of SEQ ID Nos: 79-107 and 194-206 and lacks the MPR of SEQ ID NO: 147. In some embodiments, a Siglec-9 ECD consists of an amino acid sequence selected from any one of SEQ ID Nos: 79-107 and 194-206. In some aspects, the Siglec-9 ECD is part of a Siglec-9 ECD fusion molecule, comprising the ECD and a fusion partner. In some embodiments, the fusion partner is an Fc, albumin, or PEG. In some embodiments, the fusion partner is an Fc. In some embodiments, the fusion partner is an Fe and it is located at the C-terminus of the molecule (i.e., the Fe is attached to the C-terminus of the Siglec-9 ECD either directly or via a linker). In some embodiments, the Fc is a human IgG1 (hIgG1). In some embodiments, the Fe comprises the amino acid sequence of any one of SEQ ID Nos: 142-144 and 234-239. In some embodiments, the Fc comprises the amino acid sequence of SEQ ID Nos: 142. In some embodiments, the Fc domain has an hIgG1 isotype that has: a) reduced binding to FcγRIII; b) reduced antibody-dependent cellular cytotoxicity (ATCC) and/or reduced complement binding activity; c) increased binding to FcγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fc comprises the amino acid sequence of SEQ ID No: 143. In some embodiments, the Fe is a human IgG4, with or without an S228P substitution. Thus, in some embodiments, the Fc comprises the amino acid sequence of SEQ ID NO: 145 or 146.

In some embodiments, a Siglec-9 ECD fusion molecule comprises an amino acid sequence selected from any one of SEQ ID Nos: 49-77 and 171-193, lacking a signal sequence. In some embodiments, a Siglec-9 ECD fusion molecule comprises an amino acid sequence selected from any one of SEQ ID Nos: 49-77 and 171-193, including a signal sequence. In some embodiments, a Siglec-9 ECD fusion molecule consists of an amino acid sequence selected from any one of SEQ ID Nos: 49-77 and 171-193, lacking a signal sequence. In some embodiments, a Siglec-9 ECD fusion molecule consists of an amino acid sequence selected from any one of SEQ ID Nos: 49-77 and 171-193, including a signal sequence.

In some embodiments, a Siglec-9 ECD or a Siglec-9 ECD fusion molecule comprises the amino acid sequence of SEQ ID No: 138. In some embodiments, the Siglec-9 ECD lacks the membrane proximal region (MPR) sequence of SEQ ID NO: 147. In some cases, the Siglec-9 ECD consists of the amino acid sequence of SEQ ID NO: 138. In some cases, the Siglec-9 ECD comprises or consists of the amino acid sequence of SEQ ID NO: 138 lacking the signal sequence, but wherein the Siglec-9 ECD has been expressed from a nucleic acid encoding SEQ ID NO: 138 including the signal sequence. In some cases, the Siglec-9 ECD is a Siglec-9 ECD fusion molecule comprising the ECD and a fusion partner. In some such embodiments, the fusion partner may be an Fc, albumin, or PEG. In some embodiments, the fusion partner is an Fc. In some embodiments, the fusion partner is an Fc and it is located at the C-terminus of the molecule (i.e., the Fc is attached to the C-terminus of the Siglec-9 ECD either directly or via a linker). In some embodiments, the Fc is a human IgG1 (hIgG1). In some embodiments, the Fc comprises the amino acid sequence of any one of SEQ ID Nos: 142-144 and 234-239. In some embodiments, the Fc comprises the amino acid sequence of SEQ ID NO: 142. In some embodiments, the Fc domain has an hIgG1 isotype that has: a) reduced binding to FecγRIII; b) reduced antibody-dependent cellular cytotoxicity (ATCC) and/or reduced complement binding activity; c) increased binding to FecγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fc comprises the amino acid sequence of SEQ ID NO: 143. In some embodiments, the Fe is a human IgG4, with or without an S228P substitution. Thus, in some embodiments, the Fc comprises the amino acid sequence of SEQ ID NO: 145 or 146. In some embodiments, the Siglec-9 ECD fusion molecule comprises the amino acid sequence of SEQ ID NO: 139.

In some embodiments, a Siglec-9 ECD or Siglec-9 ECD fusion molecule comprises the sequence of SEQ ID NO: 78. In some embodiments, the Siglec-9 ECD lacks the membrane proximal region sequence of SEQ ID NO: 147 (MPR). In some cases, the Siglec-9 ECD consists of the amino acid sequence of SEQ ID NO: 78. In some cases, the Siglec-9 ECD is a Siglec-9 ECD fusion molecule comprising the ECD and a fusion partner. In some such embodiments, the fusion partner may be an Fe, albumin, or PEG. In some embodiments, the fusion partner is an Fe. In some embodiments, the fusion partner is an Fe and it is located at the C-terminus of the molecule (i.e., the Fc is attached to the C-terminus of the Siglec-9 ECD either directly or via a linker). In some embodiments, the Fe is a human IgG1 (hIgG1). In some embodiments, the Fc comprises the amino acid sequence of any one of SEQ ID Nos: 142-144 and 234-239. In some embodiments, the Fc comprises the amino acid sequence of SEQ ID NO: 142. In some embodiments, the Fc domain has an hIgG1 isotype that has: a) reduced binding to FeγRIII: b) reduced antibody-dependent cellular cytotoxicity (ATCC) and/or reduced complement binding activity; c) increased binding to FeγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fe comprises the amino acid sequence of SEQ ID NO: 143. In some embodiments, the Fc is a human IgG4, with or without an S228P substitution. Thus, in some embodiments, the Fc comprises the amino acid sequence of SEQ ID NO: 145 or 146.

In some cases, a Siglec-9 ECD fusion molecule comprises the Siglec-9 ECD of SEQ ID NO: 78 joined at its C-terminus to an Fc domain or another fusion partner such as albumin or PEG, optionally via a linker or directly. In some embodiments, SEQ ID NO: 78 is directly linked at its C-terminus to an Fc domain. In some embodiments SEQ ID NO: 78 is joined at its C-terminus to an Fc domain via a linker. In some embodiments, a Siglec-9 ECD fusion molecule comprises the sequence of SEQ ID NO: 78 joined at its C-terminus to a human IgG1 or IgG4 isotype Fc domain, such as an Fc comprising any one of SEQ ID Nos: 142-144 and 234-239. In some embodiments, the Fc comprises the amino acid sequence of SEQ ID NO: 142. In some embodiments, the Fc domain has an hIgG1 isotype that has: a) reduced binding to FcγRIII; b) reduced antibody-dependent cellular cytotoxicity (ATCC) and/or reduced complement binding activity: c) increased binding to FcγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fc domain comprises SEQ ID NO: 143. In some embodiments, the Fc is a human IgG4, with or without an S228P substitution. In some embodiments, the Fc domain comprises SEQ ID NO: 145. In some embodiments, the Fc domain comprises SEQ ID NO: 146. In some embodiments, the Siglec-9 ECD fusion molecule comprises the amino acid sequence of SEQ ID NO: 10. In some embodiments, the Siglec-9 ECD fusion molecule consists of the amino acid sequence of SEQ ID NO: 10. In some embodiments, the Siglec-9 ECD fusion molecule comprises the amino acid sequence of SEQ ID NO: 227. In some embodiments, the Siglec-9 ECD fusion molecule consists of the amino acid sequence of SEQ ID NO: 227.

In some embodiments, a Siglec-9 ECD fusion molecule comprises the amino acid sequence of SEQ ID NO: 78 joined at its C-terminus to an Fc domain, wherein the molecule comprises an amino acid sequence selected from any one of SEQ ID Nos: 45-48 and 228-233, lacking its associated signal peptide. In some embodiments, a Siglec-9 ECD fusion molecule comprises the amino acid sequence of SEQ ID NO: 78 joined at its C-terminus to an Fc domain, wherein the molecule comprises an amino acid sequence selected from any one of SEQ ID Nos: 45-48 and 228-233, including its associated signal peptide. In some embodiments, the molecule comprises the amino acid sequence of SEQ ID NO: 45. In some embodiments, the molecule consists of the amino acid sequence of SEQ ID NO: 45. In some embodiments, the molecule comprises the amino acid sequence of SEQ ID NO: 48. In some embodiments, the molecule consists of the amino acid sequence of SEQ ID NO: 48. In some embodiments, the molecule comprises the amino acid sequence of SEQ ID NO: 228. In some embodiments, the molecule consists of the amino acid sequence of SEQ ID NO: 228. In some embodiments, the molecule comprises the amino acid sequence of SEQ ID NO: 229. In some embodiments, the molecule consists of the amino acid sequence of SEQ ID NO: 229. In some embodiments, the molecule comprises the amino acid sequence of SEQ ID NO: 230. In some embodiments, the molecule consists of the amino acid sequence of SEQ ID NO: 230. In some embodiments, the molecule comprises the amino acid sequence of SEQ ID NO: 231. In some embodiments, the molecule consists of the amino acid sequence of SEQ ID NO: 231. In some embodiments, the molecule comprises the amino acid sequence of SEQ ID NO: 232. In some embodiments, the molecule consists of the amino acid sequence of SEQ ID NO: 232. In some embodiments, the molecule comprises the amino acid sequence of SEQ ID NO: 233. In some embodiments, the molecule consists of the amino acid sequence of SEQ ID NO: 233.

In some embodiments, a Siglec-9 ECD comprises the sequence of SEQ ID NO: 218. In some embodiments, the Siglec-9 ECD comprises the sequence of SEQ ID NO: 198. In some embodiments, the Siglec-9 ECD comprises the sequence of SEQ ID NO: 218 or 198, and lacks the membrane proximal region (MPR) sequence of SEQ ID NO: 147. In some cases, the Siglec-9 ECD consists of the amino acid sequence of SEQ ID NO: 198. In some cases, the Siglec-9 ECD is a Siglec-9 ECD fusion molecule comprising the ECD and a fusion partner. In some such embodiments, the fusion partner may be an Fc, albumin, or PEG. In some embodiments, the fusion partner is an Fc. In some embodiments, the fusion partner is an Fc and it is located at the C-terminus of the molecule (i.e., the Fc is attached to the C-terminus of the Siglec-9 ECD cither directly or via a linker). In some embodiments, the Fc is a human IgG1 (hIgG1). In some embodiments, the Fc comprises the amino acid sequence of any one of SEQ ID Nos: 142-144 and 234-239. In some embodiments, the Fc comprises the amino acid sequence of SEQ ID NO: 142. In some embodiments, the Fc domain has an hIgG1 isotype that has: a) reduced binding to FcγRIII; b) reduced antibody-dependent cellular cytotoxicity (ATCC) and/or reduced complement binding activity: c) increased binding to FcγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fc comprises the amino acid sequence of SEQ ID NO: 143. In some embodiments, the Fc is a human IgG4, with or without an S228P substitution. Thus, in some embodiments, the Fc comprises the amino acid sequence of SEQ ID NO: 145 or 146. In some embodiments, the Siglec-9 ECD or Siglec-9 ECD fusion molecule comprises a signal sequence. In other embodiments, it does not. In some embodiments, the Siglec-9 ECD fusion molecule comprises the amino acid sequence of SEQ ID NO: 152. In some embodiments, the Siglec-9 ECD fusion molecule consists of the amino acid sequence of SEQ ID NO: 152. In some embodiments, the Siglec-9 ECD fusion molecule comprises the amino acid sequence of SEQ ID NO: 168. In some embodiments, the Siglec-9 ECD fusion molecule consists of the amino acid sequence of SEQ ID NO: 168. In some embodiments, the Siglec-9 ECD fusion molecule comprises the amino acid sequence of SEQ ID NO: 175. In some embodiments, the Siglec-9 ECD fusion molecule consists of the amino acid sequence of SEQ ID NO: 175. In some embodiments, the Siglec-9 ECD fusion molecule comprises the amino acid sequence of SEQ ID NO: 191. In some embodiments, the Siglec-9 ECD fusion molecule consists of the amino acid sequence of SEQ ID NO: 191.

In some embodiments, a Siglec-9 ECD comprises the sequence of SEQ ID NO: 219. In some embodiments, the Siglec-9 ECD comprises the sequence of SEQ ID NO: 199. In some embodiments, the Siglec-9 ECD comprises the sequence of SEQ ID NO: 219 or 199, and lacks the membrane proximal region (MPR) sequence of SEQ ID NO: 147. In some cases, the Siglec-9 ECD consists of the amino acid sequence of SEQ ID NO: 199. In some cases, the Siglec-9 ECD is a Siglec-9 ECD fusion molecule comprising the ECD and a fusion partner. In some such embodiments, the fusion partner may be an Fc, albumin, or PEG. In some embodiments, the fusion partner is an Fc. In some embodiments, the fusion partner is an Fc and it is located at the C-terminus of the molecule (i.e., the Fc is attached to the C-terminus of the Siglec-9 ECD either directly or via a linker). In some embodiments, the Fc is a human IgG1 (hIgG1). In some embodiments, the Fc comprises the amino acid sequence of any one of SEQ ID Nos: 142-144 and 234-239. In some embodiments, the Fc comprises the amino acid sequence of SEQ ID NO: 142. In some embodiments, the Fc domain has an hIgG1 isotype that has: a) reduced binding to FcγRIII: b) reduced antibody-dependent cellular cytotoxicity (ATCC) and/or reduced complement binding activity; c) increased binding to FcγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fc comprises the amino acid sequence of SEQ ID NO: 143. In some embodiments, the Fc is a human IgG4, with or without an S228P substitution. Thus, in some embodiments, the Fc comprises the amino acid sequence of SEQ ID NO: 145 or 146. In some embodiments, the Siglec-9 ECD or Siglec-9 ECD fusion molecule comprises a signal sequence. In other embodiments, it does not. In some embodiments, the Siglec-9 ECD fusion molecule comprises the amino acid sequence of SEQ ID NO: 153. In some embodiments, the Siglec-9 ECD fusion molecule consists of the amino acid sequence of SEQ ID NO: 153. In some embodiments, the Siglec-9 ECD fusion molecule comprises the amino acid sequence of SEQ ID NO: 169. In some embodiments, the Siglec-9 ECD fusion molecule consists of the amino acid sequence of SEQ ID NO: 169. In some embodiments, the Siglec-9 ECD fusion molecule comprises the amino acid sequence of SEQ ID NO: 176. In some embodiments, the Siglec-9 ECD fusion molecule consists of the amino acid sequence of SEQ ID NO: 176. In some embodiments, the Siglec-9 ECD fusion molecule comprises the amino acid sequence of SEQ ID NO: 192. In some embodiments, the Siglec-9 ECD fusion molecule consists of the amino acid sequence of SEQ ID NO: 192.

In some embodiments, a Siglec-9 ECD comprises the sequence of SEQ ID NO: 220. In some embodiments, the Siglec-9 ECD comprises the sequence of SEQ ID NO: 200. In some embodiments, the Siglec-9 ECD comprises the sequence of SEQ ID NO: 220 or 200, and lacks the membrane proximal region (MPR) sequence of SEQ ID NO: 147. In some cases, the Siglec-9 ECD consists of the amino acid sequence of SEQ ID NO: 200. In some cases, the Siglec-9 ECD is a Siglec-9 ECD fusion molecule comprising the ECD and a fusion partner. In some such embodiments, the fusion partner may be an Fc, albumin, or PEG. In some embodiments, the fusion partner is an Fe. In some embodiments, the fusion partner is an Fe and it is located at the C-terminus of the molecule (i.e., the Fe is attached to the C-terminus of the Siglec-9 ECD either directly or via a linker). In some embodiments, the Fe is a human IgG1 (hIgG1). In some embodiments, the Fc comprises the amino acid sequence of any one of SEQ ID Nos: 142-144 and 234-239. In some embodiments, the Fc comprises the amino acid sequence of SEQ ID NO: 142. In some embodiments, the Fc domain has an hIgG1 isotype that has: a) reduced binding to FcγRIII; b) reduced antibody-dependent cellular cytotoxicity (ATCC) and/or reduced complement binding activity: c) increased binding to FcγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fe comprises the amino acid sequence of SEQ ID NO: 143. In some embodiments, the Fe is a human IgG4, with or without an S228P substitution. Thus, in some embodiments, the Fe comprises the amino acid sequence of SEQ ID NO: 145 or 146. In some embodiments, the Siglec-9 ECD or Siglec-9 ECD fusion molecule comprises a signal sequence. In other embodiments, it does not. In some embodiments, the Siglec-9 ECD fusion molecule comprises the amino acid sequence of SEQ ID NO: 154. In some embodiments, the Siglec-9 ECD fusion molecule consists of the amino acid sequence of SEQ ID NO: 154. In some embodiments, the Siglec-9 ECD fusion molecule comprises the amino acid sequence of SEQ ID NO: 170. In some embodiments, the Siglec-9 ECD fusion molecule consists of the amino acid sequence of SEQ ID NO: 170. In some embodiments, the Siglec-9 ECD fusion molecule comprises the amino acid sequence of SEQ ID NO: 177. In some embodiments, the Siglec-9 ECD fusion molecule consists of the amino acid sequence of SEQ ID NO: 177. In some embodiments, the Siglec-9 ECD fusion molecule comprises the amino acid sequence of SEQ ID NO: 193. In some embodiments, the Siglec-9 ECD fusion molecule consists of the amino acid sequence of SEQ ID NO: 193.

In some embodiments, a Siglec-9 ECD fusion molecule comprises the amino acid sequence of any one of SEQ ID Nos: 207-213 joined at its C-terminus to an Fc domain. In some embodiments, the joining is direct. In other cases it is through a linker. In some embodiments, the Fc is a human IgG1 (hIgG1). In some embodiments, the Fc comprises the amino acid sequence of any one of SEQ ID Nos: 142-144 and 234-239. In some embodiments, the Fc comprises the amino acid sequence of SEQ ID NO: 142. In some embodiments, the Fc domain has an hIgG1 isotype that has: a) reduced binding to FcγRIII: b) reduced antibody-dependent cellular cytotoxicity (ATCC) and/or reduced complement binding activity: c) increased binding to FcγRIIa; or any combination of a), b), and/or c), relative to the IgG1 polypeptide of SEQ ID No: 142. In some cases, the Fc domain comprises a human IgG1 isotype with N325S and L328F (NSLF) substitutions. In some embodiments, the Fc comprises the amino acid sequence of SEQ ID NO: 143. In some embodiments, the Fc is a human IgG4, with or without an S228P substitution. Thus, in some embodiments, the Fe comprises the amino acid sequence of SEQ ID NO: 145 or 146. In some embodiments, the Siglec-9 ECD fusion molecule comprises an amino acid sequence selected from any one of SEQ ID Nos: 161-167. In some embodiments, the Siglec-9 ECD fusion molecule comprises an amino acid sequence selected from any one of SEQ ID Nos: 184-190, lacking its associated signal peptide. In some embodiments, the Siglec-9 ECD fusion molecule comprises an amino acid sequence selected from any one of SEQ ID Nos: 184-190, including its associated signal peptide.

### Exemplary Fc Domains

In some embodiments of any of the Siglec-9 ECD fusion molecules for use in methods herein, the fusion molecule may comprise an Fc domain as a fusion partner. In some embodiments, the Fc domain is a human IgG1, IgG2, IgG3, and/or IgG4 isotype.

In certain embodiments of any of the Siglec-9 ECD fusion molecules provided herein, the Fc domain has an IgG1 isotype. In some embodiments, the Siglec-9 ECD fusion molecule contains a murine IgG1 Fc domain. In some embodiments, the Siglec-9 ECD fusion molecule contains a human IgG1 Fc domain (hIgG1), e.g., as provided in SEQ ID NO: 142. In some embodiments, the human IgG1 Fc domain of the Siglec-9 ECD fusion molecule binds an activating Fc receptor. In certain embodiments, the activating Fe receptor is selected from any one or more of FcγRI, FcγRIIa and IIc, and FcγRIIIa and IIIb.

In some embodiments, the human IgG1 Fc domain of the Siglec-9 ECD fusion molecule does not bind or has reduced binding to FecγRIII (CD16) and/or C1q. In some embodiments, the human IgG1 Fc domain of the Siglec-9 ECD fusion molecule has reduced antibody-dependent cellular cytotoxicity (ADCC) and/or complement binding activity, respectively, which in each case may reduce undesired killing of cells, e.g., myeloid cells, to which the Siglec-9 ECD fusion molecule binds. The above effects may be achieved by certain amino acid modifications, e.g., the “NSLF” mutations, in which an IgG1 Fc domain contains the mutations N325S and L328F (by EU numbering of the IgG1 Fc domain), as shown, e.g., in SEQ ID NO: 143. In another embodiment, the human IgG1 Fc domain comprises a mutation corresponding to K322A (EU numbering), e.g., as provided in SEQ ID NO: 144.

Exemplary modifications to the IgG1 Fc domain are listed below in Table B.

TABLE B
Exemplary modifications to the IgG1 Fc domain
Mutation (EU numbering scheme)

N325S and L328F (“NSLF”)
S267E and L328F (“SELF”)
P331S (“PS”)
P331S and E430G (“PSEG”)
K322A
L234A, L235A, and P331S (“LALAPS”) (Substantially
abolishes Fc binding to FcR)

For example, in some embodiments, the Fc domain has a human IgG1 isotype that has: a) reduced binding to FcγRIII: b) reduced antibody-dependent cellular cytotoxicity (ATCC) and/or reduced complement binding activity: c) increased binding to FcγRIIa; or any combination of a), b), and/or c), relative to the IgG1 polypeptide of SEQ ID No: 142. In some cases, the Fc domain comprises SEQ ID NO: 143. In some cases, the Fc domain comprises a human IgG1 isotype with N325S and L328F (NSLF) substitutions.

In some embodiments, substitutions and variations can also be made in the Fc region of a Siglec-9-hIgG1 NSLF (see, e.g., SEQ ID NO:45), for example, to improve its binding to FcRn in vitro, and therefore potentially improve its ability to be recycled in vivo. Exemplary substitutions and variations include the “YTE” and “LS” substitutions, and cysteine-containing loop insertions, as described in Dall'Acqua et al. (2002) J. Immunol. 169:5171-5180; Zalevsky et al. (2010) Nat. Biotechnol. 28:157-159; and U.S. Pat. No. 9,688,756, which are each incorporated herein by reference in their entirety. In some embodiments, an Fc domain may have a sequence as shown in SEQ ID Nos: 228-230 (the substitutions and variations are indicated by double-underlined residues in the sequence table herein). Modified constructs can be tested for improved binding to FcRn in vitro, e.g., via surface plasmon resonance, and then examined for pharmacokinetics (PK) and pharmacodynamics (PD) in vivo. Modified Fc constructs may also contain the “YTE” or “LS” substitution or cysteine-containing loop insertion, but not the NSLF substitution, in the Fc. Such constructs are shown in SEQ ID Nos: 231-233.

In certain embodiments of any of the Siglec-9 ECD fusion molecules for use in methods herein, the Fc domain has an IgG2 isotype. In some embodiments, the Siglec-9 ECD fusion molecule contains a murine IgG2 Fc domain, e.g., murine IgG2a (mIgG2a). In some embodiments, the Siglec-9 ECD fusion molecule contains a human IgG2 Fc domain (hIgG2). In some embodiments, the human IgG2 Fc domain of the Siglec-9 ECD fusion molecule binds an activating Fe receptor. In certain embodiments, the activating Fe receptor is selected from any one or more of FeγRI, FeγRIIa and IIe, and FeγRIIIa and IIIb.

In certain embodiments of any of the Siglec-9 ECD fusion molecules used in methods herein, the Fc domain has an IgG4 isotype. In some embodiments, the Siglec-9 ECD fusion molecule contains a human IgG4 Fc domain (hIgG4), e.g., as provided in SEQ ID NO: 145. In some embodiments, the human IgG4 Fe region of the Siglec-9 ECD fusion molecule binds an activating Fe receptor. In certain embodiments, the activating Fc receptor is selected from any one or more of FeγRI, FeγRIIa and IIe, and FeγRIIIa and IIIb. In certain embodiments, the human IgG4 Fc region comprises a mutation corresponding to S228P (by EU numbering), e.g., as provided in SEQ ID NO: 146.

### Polypeptide Variants

In some embodiments of any of the Siglec-9 ECD polypeptides or polypeptide fusion partners, amino acid sequence variants are contemplated. For example, it may be desirable to improve the binding affinity and/or other biological properties of the polypeptide. In certain embodiments, it is also contemplated that amino acid sequence variants of Siglec-9 ECD polypeptides would maintain or exceed binding affinity of the wild type Siglec-9 ECD polypeptide.

### Substitution, Insertion, and Deletion Variants

In some embodiments, polypeptide variants having one or more amino acid substitutions are provided. Amino acid sequence variants of polypeptide may be prepared by introducing appropriate modifications into the nucleotide sequence encoding the polypeptide, or by peptide synthesis. Such modifications include, for example, deletions from, and/or insertions into and/or substitutions of residues within the amino acid sequences of the polypeptide.

TABLE C
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe: Ile | Leu |
| Phe (F) | Leu; Val: Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Modifications in the biological properties of a polypeptide may be accomplished by selecting substitutions that differ in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Scr, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human polypeptide that are homologous with non-human polypeptides, or into the non-homologous regions of the molecule.

In making changes to the polypeptide described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8): phenylalanine (+2.8); cysteine/cystine (+2.5); methionine (+1.9): alanine (+1.8): glycine (−0.4); threonine (−0.7); serine (−0.8); tryptophan (−0.9); tyrosine (−1.3): proline (−1.6): histidine (−3.2); glutamate (−3.5): glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within +2 is included. In certain embodiments, those which are within +1 are included, and in certain embodiments, those within +0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included.

Amino acid sequence insertions include amino- and/or carboxyl-terminal fusions ranging in length from one residue to polypeptides comprising a hundred or more residues, as well as intra-sequence insertions of single or multiple amino acid residues.

Any cysteine residue not involved in maintaining the proper conformation of the polypeptide also may be substituted, generally with serine, to improve the oxidative stability of the molecule and prevent aberrant crosslinking. Conversely, cysteine bond(s) may be added to a polypeptide to improve its stability.

### Other Polypeptide Modifications

In some embodiments of any of the polypeptides, the polypeptides is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified polypeptide can have a greater circulating half-life than polypeptide that is not chemically modified. In certain embodiments, a chemically modified polypeptide can have improved targeting capacity for desired cells, tissues, and/or organs. In some embodiments, a derivative polypeptide is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative polypeptide comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity of a polypeptide. Certain such methods are discussed, for example, in U.S. Pat. No. 6,133,426, which is hereby incorporated by reference for any purpose.

### Nucleic Acids, Vectors, and Host Cells

A Siglec-9 ECD or Siglec-9 ECD fusion molecule used in methods herein may be produced using recombinant methods and compositions. In some embodiments, isolated nucleic acids having a nucleotide sequence encoding any of the Siglec-9 ECD fusion molecules of the present disclosure are provided. For example, nucleic acids herein may encode a polypeptide of any one of SEQ ID Nos: 10-39, 78, 138, 148-170, and 227. Nucleic acids herein may encode an amino acid sequence selected from any one of SEQ ID Nos: 45-77, 171-193, and 228-233.

In some embodiments, a nucleic acid encodes a Siglec-9 ECD or Siglec-9 ECD fusion molecule that includes a signal sequence. In some embodiments, the signal sequence is a native signal sequence. A native human Siglec-9 signal sequence is shown in SEQ ID NO: 140. In some embodiments, the signal sequence is a non-native signal sequence. One skilled in the art would understand that any signal sequence may be used that appropriately effects intracellular trafficking of the encoded polypeptide, cleavage of the signal sequence, and secretion of the encoded polypeptide from a cell. In some such embodiments, the nucleic acid encodes a Siglec-9 ECD fusion molecule comprising a signal sequence that improves intracellular trafficking of the encoded polypeptide, signal sequence cleavage and/or secretion of the encoded polypeptide (efficiency and/or yield) relative to the native human Siglec-9 signal sequence. In some such embodiments, the nucleic acid encodes a Siglec-9 ECD fusion molecule comprising a signal sequence, wherein the signal sequence comprises the amino acid sequence of SEQ ID NO: 141. In some embodiments, a signal sequence of SEQ ID NO: 141 improves production of the Siglec-9 ECD fusion molecule.

In some embodiments, one or more nucleic acids herein may encode the amino acid sequence of SEQ ID NO: 10. In some embodiments, one or more nucleic acids herein may encode the amino acid sequence of SEQ ID NO: 45. In some embodiments, one or more nucleic acids herein may encode the amino acid sequence of SEQ ID NO: 48. In some embodiments, one or more nucleic acids herein may encode the amino acid sequence of SEQ ID NO: 138. In some embodiments, one or more nucleic acids herein may encode the amino acid sequence of SEQ ID NO: 139. In some embodiments, one or more nucleic acids herein may encode the amino acid sequence of SEQ ID NO: 227. In some embodiments, one or more nucleic acids herein may encode the amino acid sequence of SEQ ID NO: 228. In some embodiments, one or more nucleic acids herein may encode the amino acid sequence of SEQ ID NO: 229. In some embodiments, one or more nucleic acids herein may encode the amino acid sequence of SEQ ID NO: 230. In some embodiments, one or more nucleic acids herein may encode the amino acid sequence of SEQ ID NO: 231. In some embodiments, one or more nucleic acids herein may encode the amino acid sequence of SEQ ID NO: 232. In some embodiments, one or more nucleic acids herein may encode the amino acid sequence of SEQ ID NO: 233. In some embodiments, one or more nucleic acids herein may encode the amino acid sequence of SEQ ID NO: 48. In some embodiments, one or more nucleic acids herein may encode the amino acid sequence of SEQ ID NO: 198. In some embodiments, one or more nucleic acids herein may encode the amino acid sequence of SEQ ID NO: 199. In some embodiments, one or more nucleic acids herein may encode the amino acid sequence of SEQ ID NO: 200. In some embodiments, one or more nucleic acids herein may encode the amino acid sequence of SEQ ID NO: 218. In some embodiments, one or more nucleic acids herein may encode the amino acid sequence of SEQ ID NO: 219. In some embodiments, one or more nucleic acids herein may encode the amino acid sequence of SEQ ID NO: 220. In some embodiments, one or more nucleic acids herein may encode the amino acid sequence of SEQ ID NO: 152. In some embodiments, one or more nucleic acids herein may encode the amino acid sequence of SEQ ID NO: 153. In some embodiments, one or more nucleic acids herein may encode the amino acid sequence of SEQ ID NO: 154. In some embodiments, one or more nucleic acids herein may encode the amino acid sequence of SEQ ID NO: 168. In some embodiments, one or more nucleic acids herein may encode the amino acid sequence of SEQ ID NO: 169. In some embodiments, one or more nucleic acids herein may encode the amino acid sequence of SEQ ID NO: 170. In some embodiments, one or more nucleic acids herein may encode the amino acid sequence of SEQ ID NO: 175. In some embodiments, one or more nucleic acids herein may encode the amino acid sequence of SEQ ID NO: 176. In some embodiments, one or more nucleic acids herein may encode the amino acid sequence of SEQ ID NO: 177. In some embodiments, one or more nucleic acids herein may encode the amino acid sequence of SEQ ID NO: 191. In some embodiments, one or more nucleic acids herein may encode the amino acid sequence of SEQ ID NO: 192. In some embodiments, one or more nucleic acids herein may encode the amino acid sequence of SEQ ID NO: 193.

In some embodiments, any of the above nucleic acids may be incorporated into a vector for expression of the polypeptide. In some embodiments, such a vector may be incorporated into a host cell capable of expressing the polypeptide. In some embodiments, the host cell comprises (e.g., has been transduced with) a vector comprising a nucleic acid that encodes the Siglec-9 ECD fusion molecule. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells. A polypeptide may be produced from a host cell, for example, by culturing a host cell comprising a nucleic acid encoding the Siglec-9 ECD fusion molecule, under conditions suitable for expression of the Siglec-9 ECD fusion molecule. In some embodiments, the Siglec-9 ECD fusion molecule is subsequently recovered from the host cell (or host cell culture medium).

Suitable vectors comprising a nucleic acid sequence encoding any of the Siglec-9 ECD or Siglec-9 ECD fusion molecules of the present disclosure include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells include prokaryotic or eukaryotic cells. For example, Siglec-9 ECD fusion molecules of the present disclosure may be produced in eukaryotes, in particular when glycosylation and Fc effector function contribute to the activity of the molecule. In addition to prokaryotes, eukaryotic microorganisms, such as filamentous fungi or yeast, are also suitable cloning or expression hosts for Siglec-9 ECD or Siglec-9 ECD fusion molecule-encoding vectors, including fungi and yeast strains whose glycosylation pathways have been “humanized.” resulting in the production of a Siglec-9 ECD fusion molecule with a partially or fully human glycosylation pattern (e.g., Gerngross Nat. Biotech. 22:1409-1414 (2004); and Li et al. Nat. Biotech. 24:210-215 (2006)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)), which were used to recombinantly produce the Siglec-9 ECD fusion molecules of the Examples herein: baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76): human cervical carcinoma cells (HELA); canine kidney cells (MDCK; buffalo rat liver cells (BRL 3A); human lung cells (W138): human liver cells (Hep G2); mouse mammary tumor (MMT 060562): TRI cells, as described, e.g., in Mather et al. Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0.

### Exemplary Activities of Siglec-9 ECDs or Siglec-9 ECD Fusion Molecules Used in Methods Herein

In some embodiments, a polypeptide used in methods herein comprises a Siglec-9 ECD, wherein the polypeptide binds sialic acid on the surface of cells. The polypeptide in some cases may be a Siglec-9 ECD-Fc fusion molecule. A polypeptide comprising a Siglec-9 ECD may bind cells comprising sialic acid on the surface with an affinity (Kd) of less than 100 nM, or less than 90 nM, or less than 80 nM, or less than 70 nM, or less than 60 nM, or less than 50 nM, or less than 40 nM, or less than 30 nM. In some embodiments, the polypeptide binds cells comprising sialic acid on the surface with an affinity (Kd) of 0.1-100 nM, or 0.1-90 nM, or 0.1-80 nM, or 0.1-70 nM, or 0.1-60 nM, or 0.1-50 nM, or 0.1-40 nM, or 0.1-30 nM. In some embodiments, the Siglec-9 ECD or Siglec-9 ECD fusion molecule may bind to MDSCs with a Kd of, for example, less than less than 100 nM, or less than 90 nM, or less than 80 nM, or less than 70 nM, or less than 60 nM, or less than 50 nM, or less than 40 nM, or less than 30 nM, or less than 25 nM, or less than 20 nM, or less than 10 nM, or less than 5 nM, or less than 2 nM, or 0.1-50 nM, or 1-50 nM, or 1-25 nM, or 1-20 nM, or 1-10 nM, or 1-5 nM, or 1-2 nM. In various embodiments, the cells are myeloid-derived suppressor cells (MDSCs). In some cases, the MDSCs are human MDSCs.

A nonlimiting exemplary assay for determining affinity is as follows. MDSCs, such as human MDSCs, are isolated and incubated with titrating amounts of a polypeptide comprising a Siglec-9 ECD-Fc fusion molecule. A fluorescently-tagged anti-Fc domain antibody (e.g., an antibody that binds IgG1 Fc domain) is used for detection, and binding is evaluated by flow cytometry. In some embodiments, a non-human Fc domain (e.g., a mouse IgG1 Fc domain) is used in the fusion molecule, in order to reduce background binding of the fluorescently-tagged anti-Fc domain antibody to the MDSCs. An exemplary assay is provided in Example 7.

In some embodiments, a Siglec-9 ECD or Siglec-9 ECD fusion molecule repolarizes myeloid-derived suppressor cells (MDSCs). Repolarization of MDSCs may be determined, for example, by measuring increased chemokine expression from MDSCs incubated with the polypeptides. Nonlimiting exemplary chemokines whose expression may be increased, indicating repolarization of MDSCs, include CCL3, CCL4, CCL5, CCL17, CXCL1, CXCL9, and IL-8. An assay to determine repolarization may measure expression of one, two, three, four, five or more chemokines. Repolarization of MDSCs may also be determined by measuring expression of CD86 and/or CD163 expression on the MDSCs cultured in the presence of a polypeptide comprising a Siglec-9 ECD. CD86 is a pro-inflammatory marker, and an increase in CD86 expression is consistent with repolarization of MDSCs. CD163 is an M2 macrophage marker, and a decrease in CD163 expression is consistent with repolarization of MDSCs toward a pro-inflammatory phenotype.

In some embodiments, a Siglec-9 ECD or Siglec-9 ECD fusion molecule relieves MDSC-mediated suppression of T cells. A nonlimiting exemplary assay for determining relief of MDSC-mediated suppression of T cells is as follows. MDSCs are isolated and cultured, e.g., for 48 hours, with the polypeptide. The MDSCs are then co-cultured with isolated T cells (e.g., CD8+ T cells) and T-cell activator, such as Dynabeads® Human T-Activator CD3/CD28. T cell activation may be determined by measuring IFNγ expression. In some embodiments, IFNγ expression is increased, indicating T cell activation, when MDSCs are incubated with a polypeptide comprising the Siglec-9 ECD, compared to control polypeptide.

In some embodiments, a Siglec-9 ECD or Siglec-9 ECD fusion molecule blocks binding of other Siglecs to MDSCs. In some such embodiments, the polypeptide blocks binding of Siglec-3, Siglec-5, Siglec-7, Siglec-9, and/or Siglec-10 to MDSCs. Binding may be measured, for example, using the flow cytometry assay described herein for measuring Kd.

In some embodiments, a Siglec-9 ECD or Siglec-9 ECD fusion molecule may comprise the amino acid sequence of SEQ ID NO: 78 joined at its C-terminus to an Fc domain, either directly or via a linker molecule, such as the amino acid sequence of SEQ ID NO: 10 or SEQ ID NO: 227, or of any one of SEQ ID NO: 45-48 and 228-233, with or without the signal sequence. In some such cases, the molecule may bind to MDSCs, such as human MDSCs, with a Kd of, for example, less than less than 100 nM, or less than 90 nM, or less than 80 nM, or less than 70 nM, or less than 60 nM, or less than 50 nM, or less than 40 nM, or less than 30 nM, or less than 25 nM, or less than 20 nM, or less than 10 nM, or less than 5 nM, or less than 2 nM, or 0.1-50 nM, or 1-50 nM, or 1-25 nM, or 1-20 nM, or 1-10 nM, or 1-5 nM, or 1-2 nM.

For example, in some embodiments, the Fc domain has a human IgG1 isotype that has: a) reduced binding to FcγRIII; b) reduced antibody-dependent cellular cytotoxicity (ATCC) and/or reduced complement binding activity; c) increased binding to FcγRIIa; or any combination of a), b), and/or c), relative to the IgG1 polypeptide of SEQ ID No: 142. In some cases, the Fc domain comprises SEQ ID NO: 143. In some cases, the Fc domain comprises a human IgG1 isotype with N325S and L328F (NSLF) substitutions. In some such cases, such a molecule may also have increased potency in inducing IFNγ production in the presence of MDSCs compared to a Siglec-9 ECD with the same amino acid sequence, but joined at its C-terminus to an hIgG1 wild-type Fc molecule. In some embodiments, the molecule may relieve MDSC-mediated suppression of T-cells, for example, as determined by measuring an increase in IFNγ expression or an increase in T-cell proliferation. In some cases, such a molecule may increase expression of CD86 on MDSCs and/or may decrease expression of CD206 on MDSCs. In some cases, such a molecule may also bind to MDSCs, such as human MDSCs, with a Kd that is lower than that of a molecule comprising a Siglec-9 ECD of the same amino acid sequence but joined at its C-terminus to an hIgG1 wild-type Fc.

### Pharmaceutical Compositions/Formulations

The Siglec-9 ECD or Siglec-9 ECD fusion molecule used in methods herein may be provided in a pharmaceutical composition, for example, including the Siglec-9 ECD or Siglec-9 ECD fusion molecule and a pharmaceutically acceptable carrier, excipient or stabilizer (see, e.g., Remington's Pharmaceutical Sciences (1990) Mack Publishing Co., Easton, Pa; Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Acceptable carriers, excipients, or stabilizers are nontoxic to recipients at the dosages and concentrations employed.

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The present disclosure will be more fully understood by reference to the following Examples. They should not, however, be construed as limiting the scope of the present disclosure. All citations throughout the disclosure are hereby expressly incorporated by reference.

### EXAMPLES

The following examples are provided by way of illustration only and not by way of limitation. Those of skill in the art will readily recognize a variety of parameters that could be changed or modified to yield essentially similar results.

### Introduction

CD68+CD163+M2-like macrophages have been implicated in cancer-induced immune suppression. Disrupting Siglec-sialic acid signaling could confer a therapeutic benefit in cancer, particularly those cancers with high levels of myeloid-derived suppressor cells (MDSCs), which often display inhibitory Siglecs. (Santegoets et al., Cancer Immunol Immunother 68:937-979 (2019).) In the Examples herein, levels of Siglec-9, CD163, CD68 and sialic acid were assessed using IHC to identify potential predictive biomarkers for clinical use. The relevance of Siglec-9, CD163, CD68 and sialic acid to Siglec signaling and glycan immune suppression is shown in FIG. 2.

### Example 1: IHC Methods for Detecting Levels of Sialic Acid and Expression Levels of Siglec-9, CD/63, and CD68

Tissue microarrays (TriStar Technology Group, USA) with paired solid tumor samples from 432 patients with 33 tumor types were analyzed by immunohistochemistry (IHC) for CD163, CD68, and Siglec-9. Levels of sialic acid were also detected.

Samples were collected from patients with gastric cancer, lung squamous cell carcinoma, and ovarian cancer. Samples were fixed in formalin and embedded in paraffin. Sections were prepared from the FFPE (formalin-fixed paraffin-embedded) samples.

A murine anti-human Siglec-9 antibody (with murine IgG2a isotype), also called 2D4, was the primary antibody used to detect Siglec-9 in the IHC assays. 2D4 binds to the Siglec-9 extracellular domain and is described in U.S. Pat. No. 10,800,844 B2, incorporated herein by reference. A human Siglec-9 ECD fused to murine IgG1 Fc (referred to herein as “S9-mIgG1”) was used to detect sialic acid. A citrate-based pH 6.2 heat-induced epitope retrieval (HIER) was performed off-line after paraffin was removed from the FFPE sections. Citrate-based HIER was performed with S9-mIgG1 at 0.5 μg/ml and 2D4 at 5 μg/ml.

The tissue sections were stained on the Biocare intelliPATH® automated staining platform using the manufacturer's recommended settings. The sections were blocked with Biocare Peroxidase Blocker (Biocare, Cat #PX968) and Background Punisher (Biocare, Cat #BP974M) to block non-specific background.

For the detection of S9-mIgG1 or mouse primary antibodies, MACH4 HRP-polymer Detection System (Biocare. Cat #MRH534) was used. For HRP chromogenic detection, IntelliPATH® FLX DAB chromogen kit (Biocare, Cat #IPK5010) was used: an isotype control (rabbit IgG) was used under the same conditions.

For detection of CD68 by IHC, a citrate-based pH 6.2 HIER was performed to quench the non-specific antibody complexes prior to applying rabbit primary antibody against human CD68. Anti-Human CD68 rabbit monoclonal antibody (Cell Signaling, Cat #76437, Lot #1) was used at 1:400 (0.73 μg/ml) with citrate-based pH 6.2 HIER. For the detection of human CD68 rabbit primary antibodies, MACH4 AP-polymer Detection System (Biocare, Cat #MRAP536) was used. The AP chromogenic detection and counterstaining, IntelliPATH® FLX Warp Red chromogen kit (Biocare, Cat #IPK5024) and IntelliPATH® Hematoxylin (Biocare Medical, Cat #XMF963) were used. To detect CD163, anti-human CD163 rabbit monoclonal antibody D6U1J (Cell Signaling Cat #93498) was used at a concentration of 0.11 μg/ml.

The IHC scoring methodology was pre-specified, focusing on proportion of cells expressing each of CD163, CD68, and Siglec-9. Scoring was on a 4-point scale and based upon the number of cells stained in a high power field (ranging from moderate to dark staining) with the marker of interest in reasonable proximity to the tumor (tissue without tumor was not scored). An IHC score of 0 has less than 10 stained cells per high power field. An IHC score of 1 has 10-20 stained cells per high power field. An IHC score of 2 has 20-100 stained cells per high power field. An IHC score of 3 has greater than 100 stained cells per high power field.

Sequential thin sections of tissue microarrays were assessed for each of the antibody stains providing similar tumor specimen geography for comparative analyses. Immune cell sialic acid staining was estimated, referencing parallel stained sections with CD163. CD68, or CD86. CD86 staining data is not shown in the figures herein.

### Example 2: Relative Expression of Inhibitory Siglecs Across Tumor Types

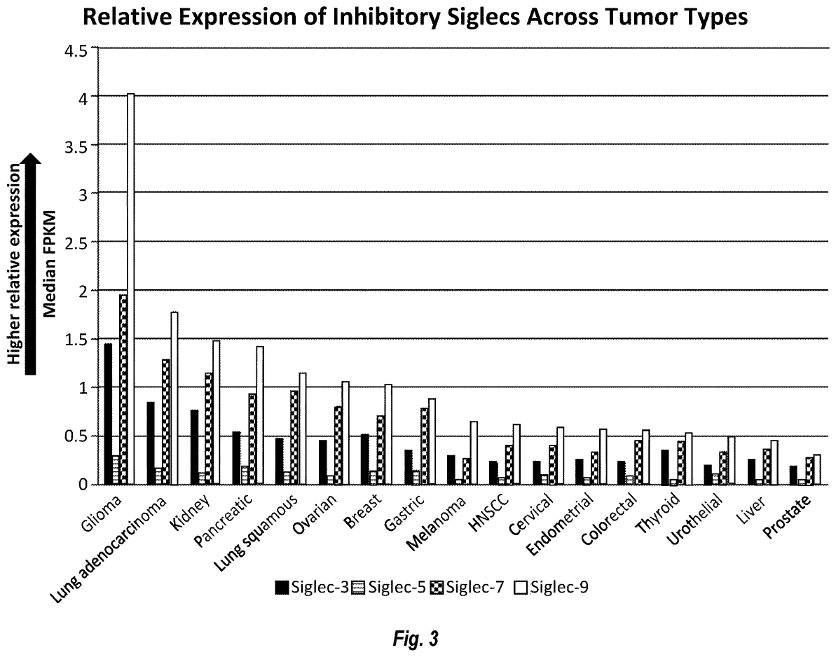

Data from the Cancer Genome Atlas (TCGA) was analyzed for the expression of four inhibitory Siglecs: Siglec-3, Siglec-5, Siglec-7, and Siglec-9. These Siglecs are typically found in myeloid cells, dendritic cells, granulocytes and microglia. Across different tumor types, Siglec-9 has higher expression levels when compared to Siglec-3. Siglec-5, and Siglec-7 (FIG. 3). The expression of each of Siglec-3. Siglec-5, Siglec-7, and Siglec-9 relative to each other appears proportionately consistent across the tumor samples (FIG. 3). Therefore, it was appropriate to use Siglec-9 expression as representative of the expression of multiple inhibitory Siglecs.

Patient tumor samples were then analyzed for levels of the Siglec ligand sialic acid, and the expression of Siglec-9, CD163, and CD68 as described in Example 1 above. CD68 is a pan-macrophage marker (i.e., present on both M1 and M2 macrophages), while CD163 is an M2-specific marker. Therefore, CD68 is often used herein as a control when IHC is performed.

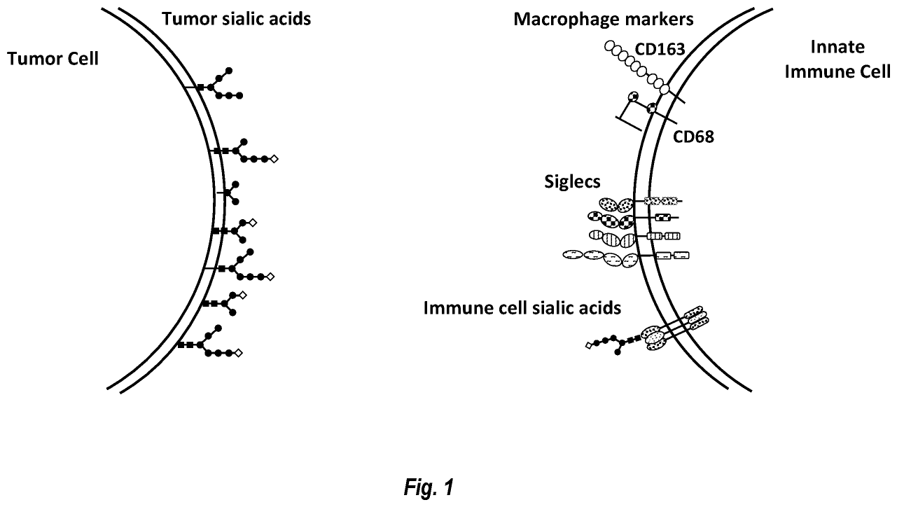

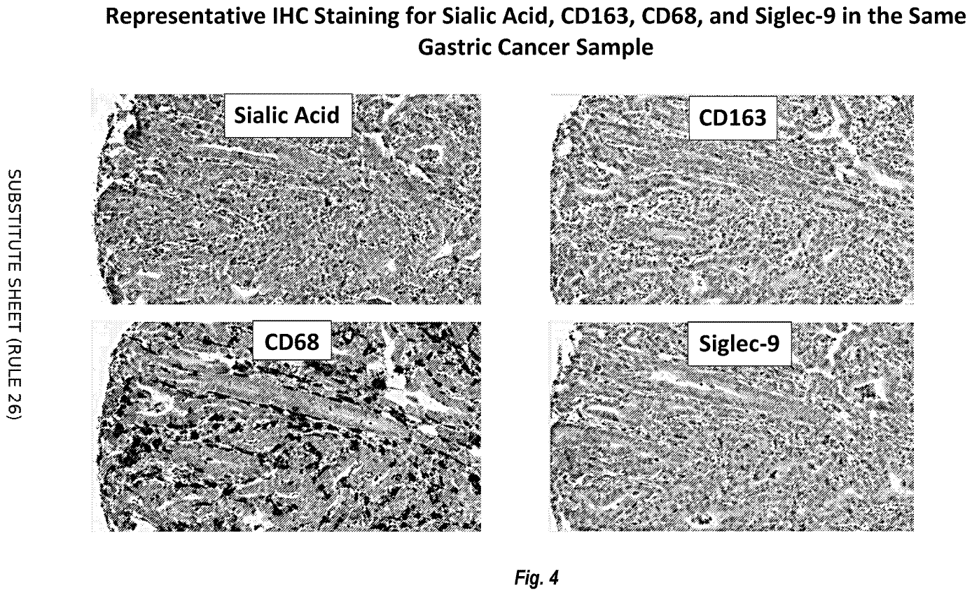

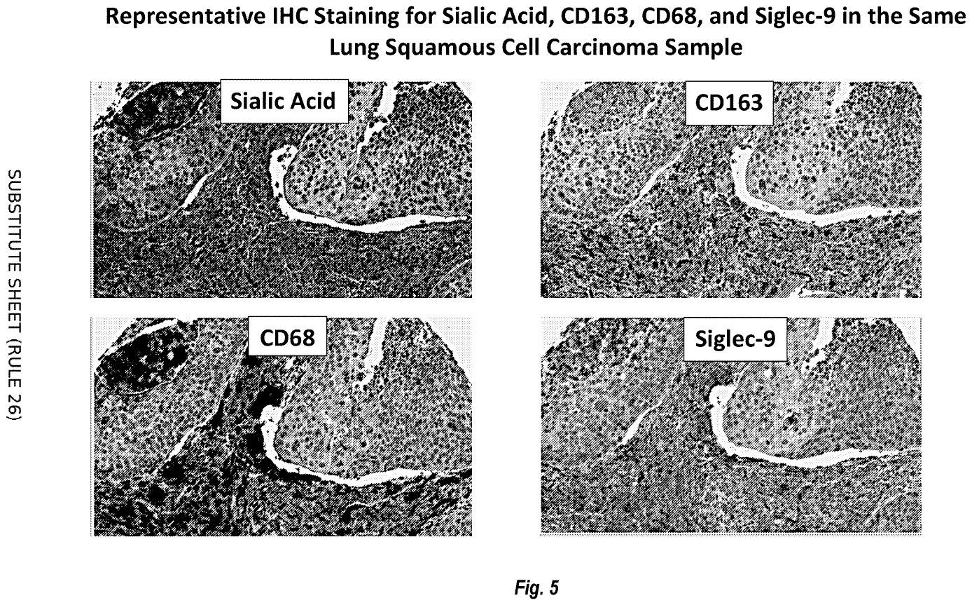

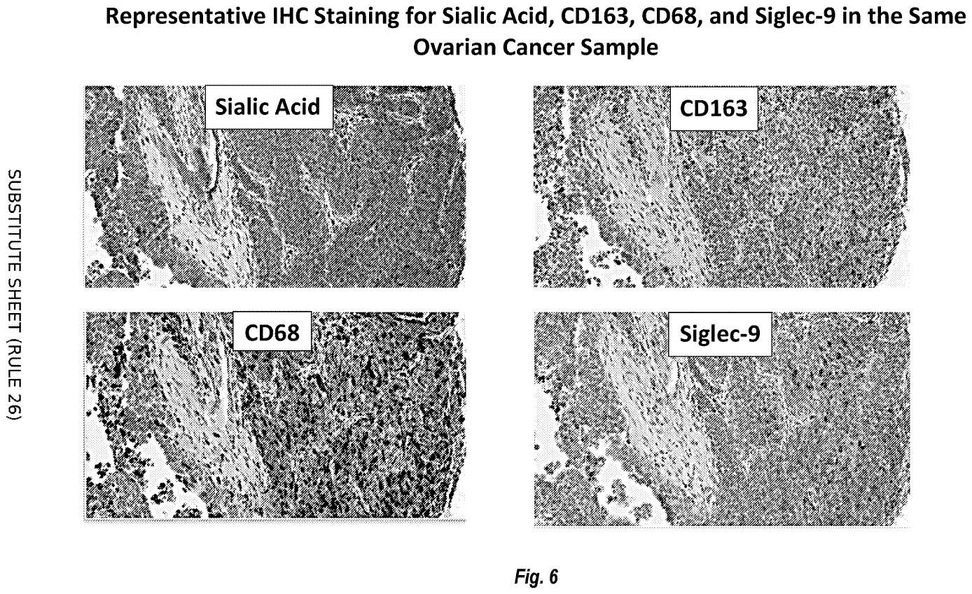

IHC showed high levels of Siglec-9, sialic acid (Siglec ligand), CD163, and CD68 (control) in gastric cancer (FIG. 4), lung squamous cell carcinoma (FIG. 5), and ovarian cancer samples (FIG. 6). The results indicate that IHC detected tumor types that are rich in MDSCs, including MDSCs with an M2 immunosuppressive phenotype. FIG. 1 (right panel) shows a schematic of such an MDSC, which expresses inhibitory Siglecs (such as Siglec-9), sialic acids, and macrophage markers (such as CD163 and CD68).

### Example 3: IHC Profiling of Tumor Types

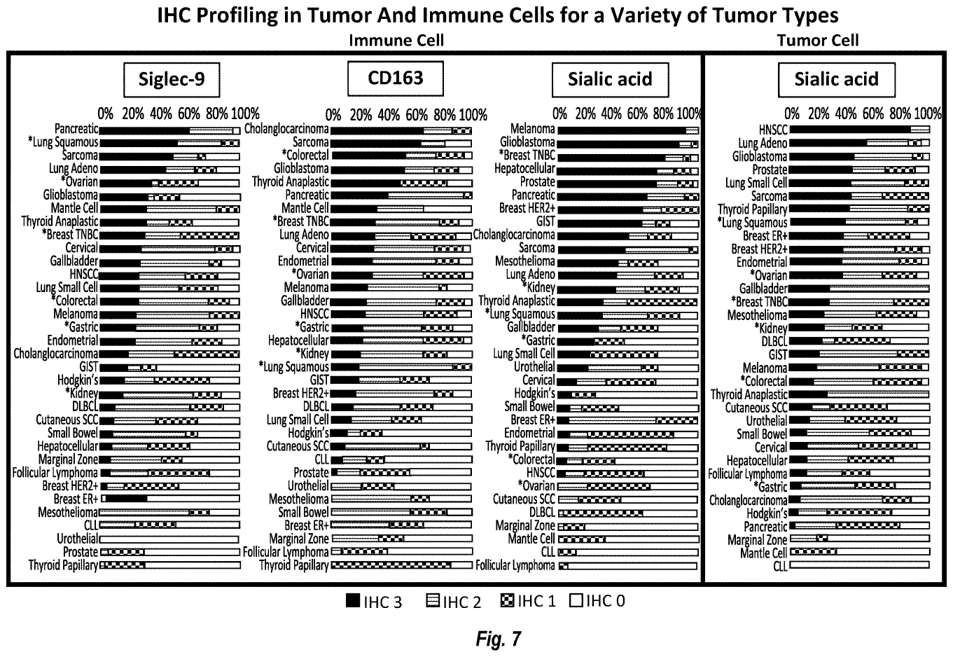

FIG. 7 shows tumor profiling of a variety of tumor types by IHC. Immune cells associated with tumors were stained for Siglec-9, CD163, and sialic acid (left panel), and tumor cells were stained for sialic acid (right panel). The left panel of FIG. 7 shows that immune cells from certain tumor types show high levels of expression of both Siglec-9 and CD163. For example, ˜70% of gastric tumors had immune cells with a score ≥IHC 2 for Siglec-9, and ˜65% had immune cells with a score of ≥IHC 2 for CD163. Similarly, ˜90% of lung (squamous) tumors had immune cells with a score ≥IHC 2 for Siglec-9, and −90% had immune cells with a score of ≥IHC 2 for CD163.

These results suggest that tumors that include immune cells with a score of at least IHC 2 for Siglec-9 would respond to an agent, such as a Siglec-9 extracellular domain (ECD) fusion polypeptide, that targets Siglec 9 or multiple Siglecs (including Siglec-9). Certain Siglec-9 extracellular domain (ECD) fusion polypeptides have been shown to repolarize MDSCs toward a pro-inflammatory phenotype, i.e., from an M2 immunosuppressive phenotype to an M1 activating phenotype, by targeting not only Siglec-9 but also other Siglecs. (See US 2021-0284710 A1, which is incorporated by reference herein.) Accordingly, such agents would be particularly suited for potentially treating tumors having immune cells that express CD163 (indicative of an M2 phenotype), as well as Siglec-9.

### Table of Certain Sequences

In the table below, bold and underlined residues in certain SEQ ID Nos show variant Siglec-9 ECD sequences represent residues that differ from the native Siglec-9 ECD sequence. Double-underlined residues in SEQ ID Nos: 228-233 show variant Fc domain residues. In some cases, residue numbers used in the name for a particular Siglec-9 variant in the “Description” column (e.g. S35X) may not match the numbering of the residues in the SEQ ID Nos of the “Sequence” column, (for example, due to the absence or presence of a signal sequence), as can be seen when comparing the bold and underlined mutated residue to its position within the SEQ ID NO below.

| SEQ ID | | |
|---|---|---|
| NO | Description | Sequence |
| 1 | Human Siglec-9 | MLLLLLPLLW GRERAEGQTS KLLTMQSSVT VQEGLCVHVP |
| | (with signal sequence) | |
| | | CSFSYPSHGW IYPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILIPGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQGVV GGAGATALVF |
| | | LSFCVIFVVV RSCRKKSARP AAGVGDTGIE DANAVRGSAS |
| | | QGPLTEPWAE DSPPDQPPPA SARSSVGEGE LQYASLSFQM |
| | | VKPWDSRGQE ATDTEYSEIK IHR |
| 2 | Mature human Siglec-9 | SKLLTMQSSV TVQEGLCVHV PCSFSYPSHG WIYPGPVVHG |
| | | YWFREGANTD QDAPVATNNP ARAVWEETRD RFHLLGDPHT |
| | | KNCTLSIRDA RRSDAGRYFF RMEKGSIKWN YKHHRLSVNV |
| | | TALTHRPNIL IPGTLESGCP QNLTCSVPWA CEQGTPPMIS |
| | | WIGTSVSPLD PSTTRSSVLT LIPQPQDHGT SLTCQVTEPG |
| | | ASVTTNKTVH LNVSYPPQNL TMTVFQGDGT VSTVLGNGSS |
| | | LSLPEGQSLR SVCAVDAVDS NPPARLSLSW RGLTLCPSQP |
| | | SNPGVLELPW VHLRDAAEFT CRAQNPLGSQ QVYLNVSLQS |
| | | KATSGVTQGV VGGAGATALV FLSFCVIFVV VRSCRKKSAR |
| | | PAAGVGDTGI EDANAVRGSA SQGPLTEPWA EDSPPDQPPP |
| | | ASARSSVGEG ELQYASLSFQ MVKPWDSRGQ EATDTEYSEI |
| | | KIHR |
| 3 | linker | ALTHR |
| 4 | linker | LNVSYP |
| 5 | ITIM motif | LQYASL |
| 6 | SLAM-like motif | TEYSEI |
| 7 | Human Siglec-9 IgV | SKLLTMQSSV TVQEGLCVHV PCSFSYPSHG WIYPGPVVHG |
| | domain | |
| | | YWFREGANTD QDAPVATNNP ARAVWEETRD RFHLLGDPHT |
| | | KNCTLSIRDA RRSDAGRYFF RMEKGSIKWN YKHHRLSVNV T |
| 8 | Siglec-7 loop | VDSQTDSD |
| 9 | Siglec-9 loop | SHGWIYPG |
| 10 | Siglec-9-Fc parental | S KLLTMQSSVT VQEGLCVHVP CSFSYPSHGW IYPGPVVHGY |
| | (WIYP (SEQ ID NO: | |
| | 254)); S9.1-hIgG1 | WFREGANTDQ DAPVATNNPA RAVWEETRDR FHLLGDPHTK |
| | (without signal | |
| | sequence) | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP SVTTNKTVHL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 11 | Siglec-9-Fc DIEG (SEQ | S KLLTMQSSVT VQEGLCVHVP CSFSYPSHGD IEGGPVVHGY |
| | ID NO: 248); S9.2- | |
| | hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 12 | Siglec-9-Fc SIET (SEQ | S KLLTMQSSVT VQEGLCVHVP CSFSYPSHGS IETGPVVHGY |
| | ID NO: 249); S9.3- | |
| | hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 13 | Siglec-9-Fc SIEP (SEQ | S KLLTMQSSVT VQEGLCVHVP CSFSYPSHGS IEPGPVVHGY |
| | ID NO: 250); S9.4- | |
| | hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 14 | Siglec-9-Fc DIEP (SEQ | S KLLTMQSSVT VQEGLCVHVP CSFSYPSHGD IEPGPVVHGY |
| | ID NO: 251); S9.5- | |
| | hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 15 | Siglec-9-Fc YQES | S KLLTMQSSVT VQEGLCVHVP CSFSYPSHGY QESGPVVHGY |
| | (SEQ ID NO: 252); | |
| | S9.6-hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 16 | Siglec-9-Fc THET | S KLLTMQSSVT VQEGLCVHVP CSFSYPSHGT HETGPVVHGY |
| | (SEQ ID NO: 253); | |
| | S9.7-hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 17 | Siglec-9-Fc L23T H26S | S KLLTMQSSVT VQEGTCVSVP CSFSYPSHGW IYPGPVVHGY |
| | H80Y L82E; S9.8- | |
| | hIgG1 | WFREGANTDQ DAPVATNNPA RAVWEETRDR FYLEGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VELFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 18 | Siglec-9-Fc L23T H26T | S KLLTMQSSVT VQEGTCVTVP CSFSYPSHGW IYPGPVVHGY |
| | H80Y L82D: S9.9- | |
| | hIgG1 | WFREGANTDQ DAPVATNNPA RAVWEETRDR FYLDGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 19 | Siglec-9-Fc S35D | S KLLTMQSSVT VQEGLCVHVP CSFSYPDHGT IYPGPVVHGY |
| | W38T; S9.10-hIgG1 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 20 | Siglec-9-Fc S35D | S KLLTMQSSVT VQEGLCVHVP CSFSYPDHGE IYPGPVVHGY |
| | W38E; S9.11-hIgG1 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 21 | Siglec-9-Fc W38S I39H | S KLLTMQSSVT VQEGLCVHVP CSFSYPSHGS HHPGPVVHGY |
| | Y40H: S9.12-hIgG1 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 22 | Siglec-9-Fc S35D | S KLLTMQSSVT VQEGLCVHVP CSFSYPDHGQ HEPGPVVHGY |
| | W38Q 139H Y40E; | |
| | S9.13-hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 23 | Siglec-9-Fc S35T | S KLLTMQSSVT VQEGLCVHVP CSFSYPTHGS HEPGPVVHGY |
| | W38Q 139H Y40E; | |
| | S9.14-hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 24 | Siglec-9-Fc S35D | S KLLTMQSSVT VQEGLCVHVP CSFSYPDHGE TYPGPVVHGY |
| | W38E I39T; S9.15- | |
| | hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 25 | Siglec-9-Fc S35N | S KLLTMQSSVT VQEGLCVHVP CSFSYPNHGT EYPGPVVHGY |
| | W38E I39T; S9.16- | |
| | hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 26 | Siglec-9-Fc S35H | S KLLTMQSSVT VQEGLCVHVP CSFSYPHHGT TTPGPVVHGY |
| | W38T 139T Y 40T; | |
| | S9.17-hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VELFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 27 | Siglec-9-Fc S35H | S KLLTMQSSVT VQEGLCVHVP CSFSYPHHGS TTPGPVVHGY |
| | W38S I39T Y40T; | |
| | S9.18-hIgG1 | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 28 | Siglec-9-Fc W38G I39T | S KLLTMQSSVT VQEGLCVHVP CSFSYPSHGG TEPGPVVHGY |
| | Y40E; S9.19-hIgG1 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 29 | Siglec-9-Fc S8D K9Y | D YTLTMQSSVT VQEGLCVHVP CSFSYPSHGW IYPGPVVHGY |
| | L10T W116E; S9.20- | |
| | hIgG1 | WFREGANTDQ DAPVATNNPA RAVWEETRDR FHLLGDPHTK |
| | | NCTLSIRDAR RSDAGRYFFR MEKGSIKENY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 30 | Siglec-9-Fc S8D K9Y | D YQLTMQSSVT VQEGLCVHVP CSFSYPSHGW IYPGPVVHGY |
| | L10Q W116N: S9.21- | |
| | hIgG1 | WFREGANTDQ DAPVATNNPA RAVWEETRDR FHLLGDPHTK |
| | | NCTLSIRDAR RSDAGRYFFR MEKGSIKNNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 31 | Siglec-9-Fc S8E K9Y | E YTLTMQSSVT VQEGLCVHVP CSFSYPSHGW IYPGPVVHGY |
| | L10T W116E; S9.22- | |
| | hIgG1 | WFREGANTDQ DAPVATNNPA RAVWEETRDR FHLLGDPHTK |
| | | NCTLSIRDAR RSDAGRYFFR MEKGSIKENY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 32 | Siglec-9-Fc WIYP | S KLLTMQSSVT VQEGLCVHVP CSFSYPSHGQ TDSGPVVHGY |
| | (SEQ ID NO: 254) to | |
| | QTDS (SEQ ID NO: | WFREGANTDQ DAPVATNNPA RAVWEETRDR FHLLGDPHTK |
| | 255); S9.23-hIgG1 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 33 | Siglec-9-Fc GWIYP | S KLLTMQSSVT VQEGLCVHVP CSFSYPSHSQ TDSGPVVHGY |
| | (SEQ ID NO: 256) to | |
| | SQTDS (SEQ ID NO: | WFREGANTDQ DAPVATNNPA RAVWEETRDR FHLLGDPHTK |
| | 257); S9.24-hIgG1 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 34 | Siglec-9-Fc | S KLLTMQSSVT VQEGLCVHVP CSFSYPVDSQ TDSDPVVHG |
| | SHGWIYPG (SEQ ID | |
| | NO:9) to VDSQTDSD | YWFREGANTD QDAPVATNNP ARAVWEETRD RFHLLGDPHT |
| | (SEQ ID NO:8); S9.25- | |
| | hIgG1 | KNCTLSIRDA RRSDAGRYFF RMEKGSIKWN YKHHRLSVNV |
| | | TALTHRPNIL IPGTLESGCP QNLTCSVPWA CEQGTPPMIS |
| | | WIGTSVSPLD PSTTRSSVLT LIPQPQDHGT SLTCQVTFPG |
| | | ASVTTNKTVH LNVSYPPQNL TMTVFQGDGT VSTVLGNGSS |
| | | LSLPEGQSLR JVCAVDAVDS NPPARLSLSW RGLTLCPSQP |
| | | SNPGVLELPW VHLRDAAEFT CRAQNPLGSQ QVYLNVSEPK |
| | | SCDKTHTCPP CPAPELLGGP SVFLFPPKPK DTLMISRTPE |
| | | VTCVVVDVSH EDPEVKENWY VDGVEVHNAK TKPREEQYNS |
| | | TYRVVSVLTV THQDWLNGKE YKCKVSNKAL PAPIEKTISK |
| | | AKGQPREPQV YTLPPSRDEL TKNQVSLTCL VKGFYPSDIA |
| | | VEWESNGQPE NNYKTTPPVL DSDGSFFLYS KLTVDKSRWQ |
| | | QGNVFSCSVM HEALHNHYTQ KSLSLSPGK |
| 35 | Siglec-9-Fc | S KLLTMQSSVT VQEGLCVHVP CSFSYPVHGQ IDSDPVVHGY |
| | SHGWIYPG (SEQ ID | |
| | NO:9) to VHGQIDSD | WFREGANTDQ DAPVATNNPA RAVWEETRDR FHLLGDPHTK |
| | (SEQ ID NO: 258); | |
| | S9.26-hIgG1 | NCTLSIRDAR RSDAGRYFFR MEKGSIKWNY KHHRLSVNVT |
| | | IGTSVSPLDP STTRSSVLTL IPQPQDHGTS LTCQVTFPGA |
| | | ALTHRPNILI PGTLESGCPQ NLTCSVPWAC EQGTPPMISW |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VELFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 36 | Siglec-9-Fc | S KLLTMQSSVT VQEGLCVHVP CSFSYPVHSQ IDSDPVVHGY |
| | SHGWIYPG (SEQ ID | |
| | NO:9) to VHSQIDSD | WFREGANTDQ STTRSSVLTL RAVWEETRDR LTCQVTFPGA |
| | (SEQ ID NO: 259); | |
| | S9.27-hIgG1 | NCTLSIRDAR DAPVATNNPA MEKGSIKWNY FHLLGDPHTK |
| | | IGTSVSPLDP RSDAGRYFFR IPQPQDHGTS KHHRLSVNVT |
| | | ALTHRPNILI PGTLESGCPQ NLTCSVPWAC EQGTPPMISW |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 37 | Siglec-9-Fc | S KLLTMQSSVT VQEGLCVHVP CSFSYPVDSQ IDSDPVVHGY |
| | SHGWIYPG (SEQ ID | |
| | NO:9) to VDSQIDSD | WFREGANTDQ DAPVATNNPA RAVWEETRDR FHLLGDPHTK |
| | (SEQ ID NO: 260); | |
| | S9.28-hIgG1 | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 38 | Siglec-9-Fc | S KLLTMQSSVT VQEGLCVHVP CSFSYPSDSQ IDSDPVVHGY |
| | SHGWIYPG (SEQ ID | |
| | NO:9) to SDSQIDSD | WFREGANTDQ DAPVATNNPA RAVWEETRDR FHLLGDPHTK |
| | (SEQ ID NO: 261); | |
| | S9.29-hIgG1 | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 39 | Siglec-9-Fc | S KLLTMQSSVT VQEGLCVHVP CSFSYPVDGQ IDSDPVVHGY |
| | SHGWIYPG (SEQ ID | |
| | NO:9) to VDGQIDSD | WFREGANTDQ DAPVATNNPA RAVWEETRDR FHLLGDPHTK |
| | (SEQ ID NO: 262); | |
| | S9.30-hIgG1 | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 40 | S9.A-hIgG1 (signal | MLLLLLPLLW GRERAEGQTS KLLTMQSSVT VQEGLCVHVP |
| | sequence is amino acids | |
| | 1-19; mature sequence | CSFSYPSHGW IYPGPVVHGY WFREGANTDQ DAPVATNNPA |
| | is amino acids 20-586) | |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQGDI EGRMDPKSCD |
| | | KTHTCPPCPA PELLGGPSVF LFPPKPKDTL MISRTPEVTC |
| | | VVVDVSHEDP EVKENWYVDG VEVHNAKTKP REEQYNSTYR |
| | | VVSVLTVLHQ DWLNGKEYKC KVSNKALPAP IEKTISKAKG |
| | | QPREPQVYTL PPSRDELTKN QVSLTCLVKG FYPSDIAVEW |
| | | ESNGQPENNY KTTPPVLDSD GSFFLYSKLT VDKSRWQQGN |
| | | VFSCSVMHEA LHNHYTQKSL SLSPGK |
| 41 | S9.A-hIgG1 NSLF | MLLLLLPLLW GRERAEGQTS KLLTMQSSVT VQEGLCVHVP |
| | (signal sequence is | |
| | amino acids 1-19; | CSFSYPSHGW IYPGPVVHGY WFREGANTDQ DAPVATNNPA |
| | mature sequence is | |
| | amino acids 20-586) | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQGDI EGRMDPKSCD |
| | | KTHTCPPCPA PELLGGPSVF LFPPKPKDTL MISRTPEVTC |
| | | VVVDVSHEDP EVKFNWYVDG VEVHNAKTKP REEQYNSTYR |
| | | VVSVLTVLHQ DWLNGKEYKC KVSSKAFPAP IEKTISKAKG |
| | | QPREPQVYTL PPSRDELTKN QVSLTCLVKG FYPSDIAVEW |
| | | ESNGQPENNY KTTPPVLDSD GSFFLYSKLT VDKSRWQQGN |
| | | VFSCSVMHEA LHNHYTQKSL SLSPGK |
| 42 | S9.A-hIgGI LALAPS | MLLLLLPLLW GRERAEGQTS KLLTMQSSVT VQEGLCVHVP |
| | (signal sequence is | |
| | amino acids 1-19; | CSFSYPSHGW IYPGPVVHGY WFREGANTDQ DAPVATNNPA |
| | mature sequence is | |
| | amino acids 20-586) | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTJ |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQGDI EGRMDPKSCD |
| | | KTHTCPPCPA PEAAGGPSVF LFPPKPKDTL MISRTPEVTC |
| | | VVVDVSHEDP EVKFNWYVDG VEVHNAKTKP REEQYNSTYR |
| | | VVSVLTVLHQ DWLNGKEYKC KVSNKALPAS IEKTISKAKG |
| | | QPREPQVYTL PPSRDELTKN QVSLTCLVKG FYPSDIAVEW |
| | | ESNGQPENNY KTTPPVLDSD GSFFLYSKLT VDKSRWQQGN |
| | | VFSCSVMHEA LHNHYTQKSL SLSPGK |
| 43 | S9.A-mIgG1 (signal | MLLLLLPLLW GRERAEGQTS KLLTMQSSVT VQEGLCVHVP |
| | sequence is amino acids | |
| | 1-19; mature sequence | CSFSYPSHGW IYPGPVVHGY WFREGANTDQ DAPVATNNPA |
| | is amino acids 20-576) | |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQGDI EGRMDCKPCI |
| | | CTVPEVSSVF IFPPKPKDVL TITLTPKVTC VVVDISKDDP |
| | | EVQFSWFVDD VEVHTAQTQP REEQENSTER SVSELPIMHQ |
| | | DWLNGKEFKC RVNSAAFPAP IEKTISKTKG RPKAPQVYTI |
| | | PPPKEQMAKD KVSLTCMITD FFPEDITVEW QWNGQPAENY |
| | | KNTQPIMNTN GSYFVYSKLN VQKSNWEAGN TFTCSVLHEG |
| | | LHNHHTEKSL SHSPGK |
| 44 | S9.B-mlgG2a (signal | MGWSCIILFL VATATGVHSS KLLTMQSSVT VQEGLCVHVP |
| | sequence is amino acids | |
| | 1-19; mature sequence | CSFSYPSHGW IYPGPVVHGY WFREGANTDQ DAPVATNNPA |
| | is amino acids 20-587) | |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQGGG GGSIEPRGPT |
| | | IKPCPPCKCP APNLLGGPSV FIFPPKIKDV LMISLSPIVT |
| | | CVVVDVSEDD PDVQISWFVN NVEVHTAQTQ THREDYNSTL |
| | | RVVSALPIQH QDWMSGKEFK CKVNNKDLPA PIERTISKPK |
| | | GSVRAPQVYV LPPPEEEMTK KQVTLTCMVT DFMPEDIYVE |
| | | WTNNGKTELN YKNTEPVLDS DGSYFMYSKL RVEKKNWVER |
| | | NSYSCSVVHE GLHNHHTTKS FSRTPGK |
| 45 | S9.1-hIgG1 NSLF | MGWSCIILFL VATATGVHSS KLLTMQSSVT VQEGLCVHVP |
| | | CSFSYPSHGW IYPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTZ |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSSKAFP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVESCSVMH EALHNHYTQK |
| | | SLSLSPGK |
| 46 | S9.1-hIgG4 S228P | MGWSCIILFL VATATGVHSS KLLTMQSSVT VQEGLCVHVP |
| | | CSFSYPSHGW IYPGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQGES KYGPPCPPCP |
| | | APEFLGGPSV FLFPPKPKDT LMISRTPEVT CVVVDVSQED |
| | | PEVQFNWYVD GVEVHNAKTK PREEQFNSTY RVVSVLTVLH |
| | | QDWLNGKEYK CKVSNKGLPS SIEKTISKAK GQPREPQVYT |
| | | LPPSQEEMTK NQVSLTCLVK GFYPSDIAVE WESNGQPENN |
| | | YKTTPPVLDS DGSFFLYSRL TVDKSRWQEG NVFSCSVMHE |
| | | ALHNHYTQKS LSLSLGK |
| 47 | S9.1-hIgG1 K322A | MGWSCIILEL VATATGVHSS KLLTMQSSVT VQEGLCVHVP |
| | | CSFSYPSHGW IYPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCAVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVESCSVMH EALHNHYTQK |
| | | SLSLSPGK |
| 48 | Signal sequence (SS)- | MGWSCIILFL VATATGVHSS KLLTMQSSVT VQEGLCVHVP |
| | Siglec-9-Fc parental | |
| | (WIYP (SEQ ID NO: | CSFSYPSHGW IYPGPVVHGY WFREGANTDQ DAPVATNNPA |
| | 254)); SS-S9.1-hIgG1 | |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 49 | SS-Siglec-9-Fc DIEG | MGWSCIILFL VATATGVHSS KLLTMQSSVT VQEGLCVHVP |
| | (SEQ ID NO: 248); SS- | |
| | S9.2-hIgG1 | CSFSYPSHGD IEGGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWINGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 50 | SS-Siglec-9-Fc SIET | MGWSCIILFL VATATGVHSS KLLTMQSSVT VQEGLCVHVP |
| | (SEQ ID NO: 249); SS- | |
| | S9.3-hIgG1 | CSFSYPSHGS IETGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVESCSVMH EALHNHYTQK |
| | | SLSLSPGK |
| 51 | SS-Siglec-9-Fc SIEP | MGWSCIILFL VATATGVHSS KLLTMQSSVT VQEGLCVHVP |
| | (SEQ ID NO: 250); SS- | |
| | S9.4-hIgG1 | CSFSYPSHGS IEPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTJ |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVESCSVMH EALHNHYTQK |
| | | SLSLSPGK |
| 52 | SS-Siglec-9-Fc DIEP | MGWSCIILFL VATATGVHSS KLLTMQSSVT VQEGLCVHVP |
| | (SEQ ID NO: 251); SS- | |
| | S9.5-hIgG1 | CSFSYPSHGD IEPGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 53 | SS-Siglec-9-Fc YQES | MGWSCIILFL VATATGVHSS KLLTMQSSVT VQEGLCVHVP |
| | (SEQ ID NO: 252); SS- | |
| | S9.6-hIgG1 | CSFSYPSHGY QESGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWINGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 54 | SS-Siglec-9-Fc THET | MGWSCIILFL VATATGVHSS KLLTMQSSVT VQEGLCVHVP |
| | (SEQ ID NO: 253); SS- | |
| | S9.7-hIgG1 | CSFSYPSHGT HETGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 55 | SS-Siglec-9-Fc L23T | MGWSCIILFL VATATGVHSS KLLTMQSSVT VQEGTCVSVP |
| | H26S H80Y L82E; SS- | |
| | S9.8-hIgG1 | CSFSYPSHGW IYPGPVVHGY WFREGANTDQ DAPVATNNPA |
| | | RAVWEETRDR FYLEGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 56 | SS-Siglec-9-Fc L23T | MGWSCIILFL VATATGVHSS KLLTMQSSVT VQEGTCVTVP |
| | H26T H80Y L82D; SS- | |
| | S9.9-hIgG1 | CSFSYPSHGW IYPGPVVHGY WFREGANTDQ DAPVATNNPA |
| | | RAVWEETRDR FYLDGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 57 | SS-Siglec-9-Fc S35D | MGWSCIILFL VATATGVHSS KLLTMQSSVT VQEGLCVHVP |
| | W38T; SS-S9.10-hIgG1 | |
| | | CSFSYPDHGT IYPGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 58 | SS-Siglec-9-Fc S35D | MGWSCIILFL VATATGVHSS KLLTMQSSVT VQEGLCVHVP |
| | W38E; SS-S9.11-hIgG1 | |
| | | CSFSYPDHGE IYPGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 59 | SS-Siglec-9-Fc W38S | MGWSCIILFL VATATGVHSS KLLTMQSSVT VQEGLCVHVP |
| | I39H Y40H; SS-S9.12- | |
| | hIgG1 | CSFSYPSHGS HHPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 60 | SS-Siglec-9-Fc S35D | MGWSCIILFL VATATGVHSS KLLTMQSSVT VQEGLCVHVP |
| | W38Q 139H Y40E: SS- | |
| | S9.13-hIgG1 | CSFSYPDHGQ HEPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS STCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVESCSVMH EALHNHYTQK |
| | | SLSLSPGK |
| 61 | SS-Siglec-9-Fc S35T | MGWSCIILFL VATATGVHSS KLLTMQSSVT VQEGLCVHVP |
| | W38Q 139H Y40E; SS- | |
| | S9.14-hIgG1 | CSFSYPTHGS HEPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 62 | SS-Siglec-9-Fc S35D | MGWSCIILFL VATATGVHSS KLLTMQSSVT VQEGLCVHVP |
| | W38E I39T; SS-S9.15- | |
| | hIgG1 | CSFSYPDHGE TYPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 63 | SS-Siglec-9-Fc S35N | MGWSCIILFL VATATGVHSS KLLTMQSSVT VQEGLCVHVP |
| | W38E I39T; SS-S9.16- | |
| | hIgG1 | CSFSYPNHGT EYPGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVESCSVMH EALHNHYTQK |
| | | SLSLSPGK |
| 64 | SS-Siglec-9-Fc S35H | MGWSCIILFL VATATGVHSS KLLTMQSSVT VQEGLCVHVP |
| | W38T 139T Y 40T; SS- | |
| | S9.17-hIgG1 | CSFSYPHHGT TTPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 65 | SS-Siglec-9-Fc S35H | MGWSCIILEL VATATGVHSS KLLTMQSSVT VQEGLCVHVP |
| | W38S 139T Y40T; SS- | |
| | S9.18-hIgG1 | CSFSYPHHGS TTPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 66 | SS-Siglec-9-Fc W38G | MGWSCIILFL VATATGVHSS KLLTMQSSVT VQEGLCVHVP |
| | I39T Y40E; SS-S9.19- | |
| | hIgG1 | CSFSYPSHGG TEPGPVVHGY WFREGANTDQ DAPVATNNPA |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVESCSVMH EALHNHYTQK |
| | | SLSLSPGK |
| 67 | SS-Siglec-9-Fc S8D | MGWSCIILFL VATATGVHSD YTLTMQSSVT VQEGLCVHVP |
| | K9Y L10T W116E; SS- | |
| | S9.20-hIgG1 | CSFSYPSHGW IYPGPVVHGY WFREGANTDQ DAPVATNNPA |
| | | RAVWEETRDR FHLLGDPHTK NCTLSIRDAR RSDAGRYFFR |
| | | MEKGSIKENY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 68 | SS-Siglec-9-Fc S8D | MGWSCIILEL VATATGVHSD YQLTMQSSVT VQEGLCVHVP |
| | K9Y L10Q W116N; | |
| | SS-S9.21-hIgG1 | CSFSYPSHGW IYPGPVVHGY WFREGANTDQ DAPVATNNPA |
| | | RAVWEETRDR FHLLGDPHTK NCTLSIRDAR RSDAGRYFFR |
| | | MEKGSIKNNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 69 | SS-Siglec-9-Fc S8E | MGWSCIILFL VATATGVHSE YTLTMQSSVT VQEGLCVHVP |
| | K9Y L10T W116E; SS- | |
| | S9.22-hIgG1 | CSFSYPSHGW IYPGPVVHGY WFREGANTDQ DAPVATNNPA |
| | | RAVWEETRDR FHLLGDPHTK NCTLSIRDAR RSDAGRYFFR |
| | | MEKGSIKENY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 70 | SS-Siglec-9-Fc WIYP | MGWSCIILFL VATATGVHSS KLLTMQSSVT VQEGLCVHVP |
| | (SEQ ID NO: 254) to | |
| | QTDS (SEQ ID NO: | CSFSYPSHGQ TDSGPVVHGY WFREGANTDQ DAPVATNNPA |
| | 255); SS-S9.23-hIgG1 | |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 71 | SS-Siglec-9-Fc GWIYP | MGWSCIILFL VATATGVHSS KLLTMQSSVT VQEGLCVHVP |
| | (SEQ ID NO: 256) to | |
| | SQTDS (SEQ ID NO: | CSFSYPSHSQ TDSGPVVHGY WFREGANTDQ DAPVATNNPA |
| | 257); SS-S9.24-hIgG1 | |
| | | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 72 | SS-Siglec-9-Fc | MGWSCIILFL VATATGVHSS KLLTMQSSVT VQEGLCVHVP |
| | SHGWIYPG (SEQ ID | |
| | NO:9) to VDSQTDSD | CSFSYPVDSQ TDSDPVVHG YWFREGANTD QDAPVATNNP |
| | (SEQ ID NO:8); SS- | |
| | S9.25-hIgG1 | ARAVWEETRD RFHLLGDPHT KNCTLSIRDA RRSDAGRYFF |
| | | RMEKGSIKWN YKHHRLSVNV TALTHRPNIL IPGTLESGCP |
| | | QNLTCSVPWA CEQGTPPMIS WIGTSVSPLD PSTTRSSVLT |
| | | LIPQPQDHGT SLTCQVTFPG ASVTTNKTVH LNVSYPPQNL |
| | | TMTVFQGDGT VSTVLGNGSS LSLPEGQSLR LVCAVDAVDS |
| | | NPPARLSLSW RGLTLCPSQP SNPGVLELPW VHLRDAAEFT |
| | | CRAQNPLGSQ QVYLNVSEPK SCDKTHTCPP CPAPELLGGP |
| | | SVFLFPPKPK DTLMISRTPE VTCVVVDVSH EDPEVKENWY |
| | | VDGVEVHNAK TKPREEQYNS TYRVVSVLTV LHQDWLNGKE |
| | | YKCKVSNKAL PAPIEKTISK AKGQPREPQV YTLPPSRDES |
| | | TKNQVSLTCL VKGFYPSDIA VEWESNGQPE NNYKTTPPVL |
| | | DSDGSFFLYS KLTVDKSRWQ QGNVFSCSVM HEALHNHYTQ |
| | | KSLSLSPGK |
| 73 | SS-Siglec-9-Fc | MGWSCIILFL VATATGVHSS KLLTMQSSVT VQEGLCVHVP |
| | SHGWIYPG (SEQ ID | |
| | NO:9) to VHGQIDSD | CSFSYPVHGQ IDSDPVVHGY WFREGANTDQ DAPVATNNPA |
| | (SEQ ID NO: 258); SS- | |
| | S9.26-hIgG1 | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 74 | SS-Siglec-9-Fc | MGWSCIILFL VATATGVHSS KLLTMQSSVT VQEGLCVHVP |
| | SHGWIYPG (SEQ ID | |
| | NO:9) to VHSQIDSD | CSFSYPVHSQ IDSDPVVHGY WFREGANTDQ DAPVATNNPA |
| | (SEQ ID NO: 259); SS- | |
| | S9.27-hIgG1 | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWINGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 75 | SS-Siglec-9-Fc | MGWSCIILFL VATATGVHSS KLLTMQSSVT VQEGLCVHVP |
| | SHGWIYPG (SEQ ID | |
| | NO:9) to VDSQIDSD | CSFSYPVDSQ IDSDPVVHGY WFREGANTDQ DAPVATNNPA |
| | (SEQ ID NO: 260); SS- | |
| | S9.28-hIgG1 | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWINGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 76 | SS-Siglec-9-Fc | MGWSCIILFL VATATGVHSS KLLTMQSSVT VQEGLCVHVP |
| | SHGWIYPG (SEQ ID | |
| | NO:9) to SDSQIDSD | CSFSYPSDSQ IDSDPVVHGY WFREGANTDQ DAPVATNNPA |
| | (SEQ ID NO: 261); SS- | |
| | S9.29-hIgG1 | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWINGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 77 | SS-Siglec-9-Fc | MGWSCIILFL VATATGVHSS KLLTMQSSVT VQEGLCVHVP |
| | SHGWIYPG (SEQ ID | |
| | NO:9) to VDGQIDSD | CSFSYPVDGQ IDSDPVVHGY WFREGANTDQ DAPVATNNPA |
| | (SEQ ID NO: 262); SS- | |
| | S9.30-hIgG1 | RAVWEETRDR FHLLGDPHTK NCTLSIRDAR RSDAGRYFFR |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWINGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 78 | Siglec-9 parental | S KLLTMQSSVT VQEGLCVHVP CSFSYPSHGW IYPGPVVHGY |
| | (WIYP(SEQ ID NO: | |
| | 254)); S9.1 (without | WFREGANTDQ DAPVATNNPA RAVWEETRDR FHLLGDPHTK |
| | signal sequence) | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 79 | Siglec-9 DIEG (SEQ ID | S KLLTMQSSVT VQEGLCVHVP CSFSYPSHGD IEGGPVVHGY |
| | NO: 248); S9.2 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 80 | Siglec-9 SIET (SEQ ID | S KLLTMQSSVT VQEGLCVHVP CSFSYPSHGS IETGPVVHGY |
| | NO: 249); S9.3 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 81 | Siglec-9 SIEP (SEQ ID | S KLLTMQSSVT VQEGLCVHVP CSFSYPSHGS IEPGPVVHGY |
| | NO: 250); S9.4 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 82 | Siglec-9 DIEP (SEQ ID | S KLLTMQSSVT VQEGLCVHVP CSFSYPSHGD IEPGPVVHGY |
| | NO: 251); S9.5 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 83 | Siglec-9 YQES (SEQ | S KLLTMQSSVT VQEGLCVHVP CSFSYPSHGY QESGPVVHGY |
| | ID NO: 252); S9.6 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 84 | Siglec-9 THET (SEQ | S KLLTMQSSVT VQEGLCVHVP CSFSYPSHGT HETGPVVHGY |
| | ID NO: 253); S9.7 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 85 | Siglec-9 L23T H26S | S KLLTMQSSVT VQEGTCVSVP CSFSYPSHGW IYPGPVVHGY |
| | H80Y L82E; S9.8 | |
| | | WFREGANTDQ DAPVATNNPA RAVWEETRDR FYLEGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSZ |
| | | SLPEGQSLRL VCAVDAVDSN PPARLSLSWR GLTLCPSQPS |
| | | NPGVLELPWV HLRDAAEFTC RAQNPLGSQQ VYLNVS |
| 86 | Siglec-9 L23T H26T | S KLLTMQSSVT VQEGTCVTVP CSFSYPSHGW IYPGPVVHGY |
| | H80Y L82D; S9.9 | |
| | | WFREGANTDQ DAPVATNNPA RAVWEETRDR FYLDGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVS |
| 87 | Siglec-9 S35D W38T; | S KLLTMQSSVT VQEGLCVHVP CSFSYPDHGT IYPGPVVHGY |
| | S9.10 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 88 | Siglec-9 S35D W38E; | S KLLTMQSSVT VQEGLCVHVP CSFSYPDHGE IYPGPVVHGY |
| | S9.11 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 89 | Siglec-9 W38S I39H | S KLLTMQSSVT VQEGLCVHVP CSFSYPSHGS HHPGPVVHGY |
| | Y40H; S9.12 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 90 | Siglec-9 S35D W38Q | S KLLTMQSSVT VQEGLCVHVP CSFSYPDHGQ HEPGPVVHGY |
| | I39H Y40E; S9.13 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 91 | Siglec-9 S35T W38Q | S KLLTMQSSVT VQEGLCVHVP CSFSYPTHGS HEPGPVVHGY |
| | I39H Y40E; S9.14 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 92 | Siglec-9 S35D W38E | S KLLTMQSSVT VQEGLCVHVP CSFSYPDHGE TYPGPVVHGY |
| | I39T; S9.15 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 93 | Siglec-9 S35N W38E | S KLLTMQSSVT VQEGLCVHVP CSFSYPNHGT EYPGPVVHGY |
| | I39T; S9.16 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 94 | Siglec-9 S35H W38T | S KLLTMQSSVT VQEGLCVHVP CSFSYPHHGT TTPGPVVHGY |
| | I39T Y40T; S9.17 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 95 | Siglec-9 S35H W38S | S KLLTMQSSVT VQEGLCVHVP CSFSYPHHGS TTPGPVVHGY |
| | I39T Y40T; S9.18 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVS |
| 96 | Siglec-9 W38G I39T | S KLLTMQSSVT VQEGLCVHVP CSFSYPSHGG TEPGPVVHGY |
| | Y40E; S9.19 | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 97 | Siglec-9 S8D K9Y | D YTLTMQSSVT VQEGLCVHVP CSFSYPSHGW IYPGPVVHGY |
| | L10T W116E; S9.20 | |
| | | WFREGANTDQ DAPVATNNPA RAVWEETRDR FHLLGDPHTK |
| | | NCTLSIRDAR RSDAGRYFFR MEKGSIKENY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 98 | Siglec-9 S8D K9Y | D YQLTMQSSVT VQEGLCVHVP CSFSYPSHGW IYPGPVVHGY |
| | L10Q W116N; S9.21 | |
| | | WFREGANTDQ DAPVATNNPA RAVWEETRDR FHLLGDPHTK |
| | | NCTLSIRDAR RSDAGRYFFR MEKGSIKNNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 99 | Siglec-9 S8E K9Y | E YTLTMQSSVT VQEGLCVHVP CSFSYPSHGW IYPGPVVHGY |
| | L10T W116E; S9.22 | |
| | | WFREGANTDQ DAPVATNNPA RAVWEETRDR FHLLGDPHTK |
| | | NCTLSIRDAR RSDAGRYFFR MEKGSIKENY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSS |
| | | SLPEGQSLRL VCAVDAVDSN PPARLSLSWR GLTLCPSQPS |
| | | NPGVLELPWV HLRDAAEFTC RAQNPLGSQQ VYLNVS |
| 100 | Siglec-9 WIYP (SEQ | S KLLTMQSSVT VQEGLCVHVP CSFSYPSHGQ TDSGPVVHGY |
| | ID NO: 254) to QTDS | |
| | (SEQ ID NO: 255); | WFREGANTDQ DAPVATNNPA RAVWEETRDR FHLLGDPHTK |
| | S9.23 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 101 | Siglec-9 GWIYP (SEQ | S KLLTMQSSVT VQEGLCVHVP CSFSYPSHSQ TDSGPVVHGY |
| | ID NO: 256) to SQTDS | |
| | (SEQ ID NO: 257); | WFREGANTDQ DAPVATNNPA RAVWEETRDR FHLLGDPHTK |
| | S9.24 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 102 | Siglec-9 SHGWIYPG | S KLLTMQSSVT VQEGLCVHVP CSFSYPVDSQ TDSDPVVHG |
| | (SEQ ID NO:9) to | |
| | VDSQTDSD (SEQ ID | YWFREGANTD QDAPVATNNP ARAVWEETRD RFHLLGDPHT |
| | NO:8); S9.25 | |
| | | KNCTLSIRDA RRSDAGRYFF RMEKGSIKWN YKHHRLSVNV |
| | | TALTHRPNIL IPGTLESGCP QNLTCSVPWA CEQGTPPMIS |
| | | WIGTSVSPLD PSTTRSSVLT LIPQPQDHGT SLTCQVTEPG |
| | | ASVTTNKTVH LNVSYPPQNL TMTVFQGDGT VSTVLGNGSS |
| | | LSLPEGQSLR LVCAVDAVDS NPPARLSLSW RGLTLCPSQP |
| | | SNPGVLELPW VHLRDAAEFT CRAQNPLGSQ QVYLNVS |
| 103 | Siglec-9 SHGWIYPG | S KLLTMQSSVT VQEGLCVHVP CSFSYPVHGQ IDSDPVVHGY |
| | (SEQ ID NO:9) to | |
| | VHGQIDSD (SEQ ID | WFREGANTDQ DAPVATNNPA RAVWEETRDR FHLLGDPHTK |
| | NO: 258); S9.26 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 104 | Siglec-9 SHGWIYPG | S KLLTMQSSVT VQEGLCVHVP CSFSYPVHSQ IDSDPVVHGY |
| | (SEQ ID NO:9) to | |
| | VHSQIDSD (SEQ ID | WFREGANTDQ DAPVATNNPA RAVWEETRDR FHLLGDPHTK |
| | NO: 259); S9.27 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 105 | Siglec-9 SHGWIYPG | S KLLTMQSSVT VQEGLCVHVP CSFSYPVDSQ IDSDPVVHGY |
| | (SEQ ID NO:9) to | |
| | VDSQIDSD (SEQ ID | WFREGANTDQ DAPVATNNPA RAVWEETRDR FHLLGDPHTK |
| | NO: 260); S9.28 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 106 | Siglec-9 SHGWIYPG | S KLLTMQSSVT VQEGLCVHVP CSFSYPSDSQ IDSDPVVHGY |
| | (SEQ ID NO:9) to | |
| | SDSQIDSD (SEQ ID | WFREGANTDQ DAPVATNNPA RAVWEETRDR FHLLGDPHTK |
| | NO: 261); S9.29 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTEPGA |
| | | SVTTNKTVHL NVSYPPQNLT MTVFQGDGTV STVLGNGSSZ |
| | | SLPEGQSLRL VCAVDAVDSN PPARLSLSWR GLTLCPSQPS |
| | | NPGVLELPWV HLRDAAEFTC RAQNPLGSQQ VYLNVS |
| 107 | Siglec-9 SHGWIYPG | S KLLTMQSSVT VQEGLCVHVP CSFSYPVDGQ IDSDPVVHGY |
| | (SEQ ID NO:9) to | |
| | VDGQIDSD (SEQ ID | WFREGANTDQ DAPVATNNPA RAVWEETRDR FHLLGDPHTK |
| | NO: 262); S9.30 | |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVS |
| 108 | Siglec-9 parental | S KLLTMQSSVT VQEGLCVHVP CSFSYPSHGW IYPGPVVHGY |
| | (WIYP (SEQ ID NO: | |
| | 254)) IgV domain; | WFREGANTDQ DAPVATNNPA RAVWEETRDR FHLLGDPHTK |
| | S9.1-IgV (without | |
| | signal sequence) | NCTLSIRDAR RSDAGRYFFR MEKGSIKWNY KHHRLSVNVT |
| 109 | Siglec-9 DIEG (SEQ ID | S KLLTMQSSVT VQEGLCVHVP CSFSYPSHGD IEGGPVVHGY |
| | NO: 248) IgV domain; | |
| | S9.2-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 110 | Siglec-9 SIET (SEQ ID | S KLLTMQSSVT VQEGLCVHVP CSFSYPSHGS IETGPVVHGY |
| | NO: 249) IgV domain; | |
| | S9.3-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 111 | Siglec-9 SIEP (SEQ ID | S KLLTMQSSVT VQEGLCVHVP CSFSYPSHGS IEPGPVVHGY |
| | NO: 250) IgV domain; | |
| | S9.4-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 112 | Siglec-9 DIEP (SEQ ID | S KLLTMQSSVT VQEGLCVHVP CSFSYPSHGD IEPGPVVHGY |
| | NO: 251)IgV domain; | |
| | S9.5-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 113 | Siglec-9 YQES (SEQ | S KLLTMQSSVT VQEGLCVHVP CSFSYPSHGY QESGPVVHGY |
| | ID NO: 252) IgV | |
| | domain; S9.6-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 114 | Siglec-9 THET (SEQ | S KLLTMQSSVT VQEGLCVHVP CSFSYPSHGT HETGPVVHGY |
| | ID NO: 253) IgV | |
| | domain; S9.7-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 115 | Siglec-9 L23T H26S | S KLLTMQSSVT VQEGTCVSVP CSFSYPSHGW IYPGPVVHGY |
| | H80Y L82E IgV | |
| | domain; S9.8-IgV | WFREGANTDQ DAPVATNNPA RAVWEETRDR FYLEGDPHTK |
| | | NCTLSIRDAR RSDAGRYFFR MEKGSIKWNY KHHRLSVNVT |
| 116 | Siglec-9 L23T H26T | S KLLTMQSSVT VQEGTCVTVP CSFSYPSHGW IYPGPVVHGY |
| | H80Y L82D IgV | |
| | domain; S9.9-IgV | WFREGANTDQ DAPVATNNPA RAVWEETRDR FYLDGDPHTK |
| | | NCTLSIRDAR RSDAGRYFFR MEKGSIKWNY KHHRLSVNVT |
| 117 | Siglec-9 S35D W38T | S KLLTMQSSVT VQEGLCVHVP CSFSYPDHGT IYPGPVVHGY |
| | IgV domain; S9.10-IgV | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| 118 | Siglec-9 S35D W38E | S KLLTMQSSVT VQEGLCVHVP CSFSYPDHGE IYPGPVVHGY |
| | IgV domain; S9.11-IgV | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| 119 | Siglec-9 W38S 139H | S KLLTMQSSVT VQEGLCVHVP CSFSYPSHGS HHPGPVVHGY |
| | Y40H IgV domain; | |
| | S9.12-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 120 | Siglcc-9 S35D W38Q | S KLLTMQSSVT VQEGLCVHVP CSFSYPDHGQ HEPGPVVHGY |
| | 139H Y40E IgV | |
| | domain; S9.13-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 121 | Siglec-9 S35T W38Q | S KLLTMQSSVT VQEGLCVHVP CSFSYPTHGS HEPGPVVHGY |
| | I39H Y40E IgV | |
| | domain; S9.14-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 122 | Siglec-9 S35D W38E | S KLLTMQSSVT VQEGLCVHVP CSFSYPDHGE TYPGPVVHGY |
| | 139T IgV domain; | |
| | S9.15-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 123 | Siglec-9 S35N W38E | S KLLTMQSSVT VQEGLCVHVP CSFSYPNHGT EYPGPVVHGY |
| | I39T IgV domain; | |
| | S9.16-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 124 | Siglec-9 S35H W38T | S KLLTMQSSVT VQEGLCVHVP CSFSYPHHGT TTPGPVVHGY |
| | I39T Y40T IgV domain; | |
| | S9.17-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 125 | Siglec-9 S35H W38S | S KLLTMQSSVT VQEGLCVHVP CSFSYPHHGS TTPGPVVHGY |
| | I39T Y40T IgV domain; | |
| | S9.18-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 126 | Siglcc-9 W38G I39T | S KLLTMQSSVT VQEGLCVHVP CSFSYPSHGG TEPGPVVHGY |
| | Y40E IgV domain; | |
| | S9.19-IgV | WFREGANTDQ DAPVATNNPA RAVWEETRDR FHLLGDPHTK |
| | | NCTLSIRDAR RSDAGRYFFR MEKGSIKWNY KHHRLSVNVT |
| 127 | Siglec-9 S8D K9Y | D YTLTMQSSVT VQEGLCVHVP CSFSYPSHGW IYPGPVVHGY |
| | L10T W116E IgV | |
| | domain; S9.20-IgV | WFREGANTDQ DAPVATNNPA RAVWEETRDR FHLLGDPHTK |
| | | NCTLSIRDAR RSDAGRYFFR MEKGSIKENY KHHRLSVNVT |
| 128 | Siglcc-9 S8D K9Y | D YQLTMQSSVT VQEGLCVHVP CSFSYPSHGW IYPGPVVHGY |
| | L10Q W116N IgV | |
| | domain; S9.21-IgV | WFREGANTDQ DAPVATNNPA RAVWEETRDR FHLLGDPHTK |
| | | NCTLSIRDAR RSDAGRYFFR MEKGSIKNNY KHHRLSVNVT |
| 129 | Siglec-9 S8E K9Y | E YTLTMQSSVT VQEGLCVHVP CSFSYPSHGW IYPGPVVHGY |
| | L10T W116E IgV | |
| | domain; S9.22-IgV | WFREGANTDQ DAPVATNNPA RAVWEETRDR FHLLGDPHTK |
| | | NCTLSIRDAR RSDAGRYFFR MEKGSIKENY KHHRLSVNVT |
| 130 | Siglcc-9 WIYP (SEQ | S KLLTMQSSVT VQEGLCVHVP CSFSYPSHGQ TDSGPVVHGY |
| | ID NO: 254) to QTDS | |
| | (SEQ ID NO: 255) IgV | WFREGANTDQ DAPVATNNPA RAVWEETRDR FHLLGDPHTK |
| | domain; S9.23-IgV | |
| | | NCTLSIRDAR RSDAGRYFFR MEKGSIKWNY KHHRLSVNVT |
| 131 | Siglec-9 GWIYP (SEQ | S KLLTMQSSVT VQEGLCVHVP CSFSYPSHSQ TDSGPVVHGY |
| | ID NO: 256) to SQTDS | |
| | (SEQ ID NO: 257) IgV | WFREGANTDQ DAPVATNNPA RAVWEETRDR FHLLGDPHTK |
| | domain; S9.24-IgV | |
| | | NCTLSIRDAR RSDAGRYFFR MEKGSIKWNY KHHRLSVNVT |
| 132 | Siglec-9 SHGWIYPG | S KLLTMQSSVT VQEGLCVHVP CSFSYPVDSQ TDSDPVVHG |
| | (SEQ ID NO:9) to | |
| | VDSQTDSD (SEQ ID | YWFREGANTD QDAPVATNNP ARAVWEETRD RFHLLGDPHT |
| | NO:8) IgV domain; | |
| | S9.25-IgV | KNCTLSIRDA RRSDAGRYFF RMEKGSIKWN YKHHRLSVNV T |
| 133 | Siglcc-9 SHGWIYPG | S KLLTMQSSVT VQEGLCVHVP CSFSYPVHGQ IDSDPVVHGY |
| | (SEQ ID NO:9) to | |
| | VHGQIDSD (SEQ ID | WFREGANTDQ DAPVATNNPA RAVWEETRDR FHLLGDPHTK |
| | NO: 258) IgV domain; | |
| | S9.26-Ig V | NCTLSIRDAR RSDAGRYFFR MEKGSIKWNY KHHRLSVNVT |
| 134 | Siglec-9 SHGWIYPG | S KLLTMQSSVT VQEGLCVHVP CSFSYPVHSQ IDSDPVVHGY |
| | (SEQ ID NO:9) to | |
| | VHSQIDSD (SEQ ID | WFREGANTDQ DAPVATNNPA RAVWEETRDR FHLLGDPHTK |
| | NO: 259) IgV domain; | |
| | S9.27-IgV | NCTLSIRDAR RSDAGRYFFR MEKGSIKWNY KHHRLSVNVT |
| 135 | Siglec-9 SHGWIYPG | S KLLTMQSSVT VQEGLCVHVP CSFSYPVDSQ IDSDPVVHGY |
| | (SEQ ID NO:9) to | |
| | VDSQIDSD (SEQ ID | WFREGANTDQ DAPVATNNPA RAVWEETRDR FHLLGDPHTK |
| | NO: 260) IgV domain; | |
| | S9.28-IgV | NCTLSIRDAR RSDAGRYFFR MEKGSIKWNY KHHRLSVNVT |
| 136 | Siglec-9 SHGWIYPG | S KLLTMQSSVT VQEGLCVHVP CSFSYPSDSQ IDSDPVVHGY |
| | (SEQ ID NO:9) to | |
| | SDSQIDSD (SEQ ID | WFREGANTDQ DAPVATNNPA RAVWEETRDR FHLLGDPHTK |
| | NO: 261) IgV domain; | |
| | S9.29-IgV | NCTLSIRDAR RSDAGRYFFR MEKGSIKWNY KHHRLSVNVT |
| 137 | Siglec-9 SHGWIYPG | S KLLTMQSSVT VQEGLCVHVP CSFSYPVDGQ IDSDPVVHGY |
| | (SEQ ID NO:9) to | |
| | VDGQIDSD (SEQ ID | WFREGANTDQ DAPVATNNPA RAVWEETRDR FHLLGDPHTK |
| | NO: 262) IgV domain; | |
| | S9.30-IgV | NCTLSIRDAR RSDAGRYFFR MEKGSIKWNY KHHRLSVNVT |
| 138 | SSopt-S9.A ECD (with | MGWSCIILFL VATATGVHSS KLLTMQSSVT VQEGLCVHVP |
| | optimized signal | |
| | sequence) | CSFSYPSHGW IYPGPVVHGY WFREGANTDQ DAPVATNNPA |
| | | RAVWEETRDR FHLLGDPHTK NCTLSIRDAR RSDAGRYFER |
| | | MEKGSIKWNY KHHRLSVNVT ALTHRPNILI PGTLESGCPQ |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | RAQNPLGSQQ VYLNVSLQSK ATSGVTQG |
| 139 | SSopt-S9.A ECD- | MGWSCIILEL VATATGVHSS KLLTMQSSVT VQEGLCVHVP |
| | hIgG1 (with optimized | |
| | signal sequence from | CSFSYPSHGW IYPGPVVHGY WFREGANTDQ DAPVATNNPA |
| | aa | |
| | 1-19 and linker | RAVWEETRDR FHLLGDPHTK NCTLSIRDAR RSDAGRYFFR |
| | sequence (boxed) | |
| | between ECD and | MEKGSIKWNY KHHRLSVNVT ALTHRPNILI PGTLESGCPQ |
| | hIgG1 Fc domain) | |
| | | NLTCSVPWAC EQGTPPMISW IGTSVSPLDP STTRSSVLTL |
| | | IPQPQDHGTS LTCQVTFPGA SVTTNKTVHL NVSYPPQNLT |
| | | MTVFQGDGTV STVLGNGSSL SLPEGQSLRL VCAVDAVDSN |
| | | PPARLSLSWR GLTLCPSQPS NPGVLELPWV HLRDAAEFTC |
| | | 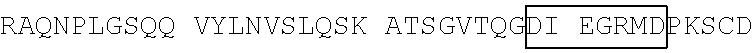 |
| | | KTHTCPPCPA PELLGGPSVF LFPPKPKDTL MISRTPEVTC |
| | | VVVDVSHEDP EVKENWYVDG VEVHNAKTKP REEQYNSTYR |
| | | VVSVLTVLHQ DWLNGKEYKC KVSNKALPAP IEKTISKAKG |
| | | QPREPQVYTL PPSRDELTKN QVSLTCLVKG FYPSDIAVEW |
| | | ESNGQPENNY KTTPPVLDSD GSFFLYSKLT VDKSRWQQGN |
| | | VFSCSVMHEA SHNHYTQKSL SLSPGK |
| 140 | Native signal sequence | MLLLLLPLLW GRERAEGQT |
| 141 | Optimized signal | MGWSCIILFL VATATGVHS |
| | sequence | |
| 142 | IgG1 wild-type | EPKS CDKTHTCPPC PAPELLGGPS VFLFPPKPKD |
| | | TLMISRTPEV TCVVVDVSHE DPEVKENWYV DGVEVHNAKT |
| | | KPREEQYNST YRVVSVLTVL HQDWLNGKEY KCKVSNKALP |
| | | APIEKTISKA KGQPREPQVY TLPPSRDELT KNQVSLTCLV |
| | | KGFYPSDIAV EWESNGQPEN NYKTTPPVLD SDGSFFLYSK |
| | | LTVDKSRWQQ GNVFSCSVMH EALHNHYTQK SLSLSPGK |
| 143 | IgG1 NSLF (N to S and | EPKS CDKTHTCPPC PAPELLGGPS VFLFPPKPKD |
| | L to F substitutions | |
| | underlined) | TLMISRTPEV TCVVVDVSHE DPEVKENWYV DGVEVHNAKT |
| | | KPREEQYNST YRVVSVLTVL HQDWLNGKEY KCKVSSKAFP |
| | | APIEKTISKA KGQPREPQVY TLPPSRDELT KNQVSLTCLV |
| | | KGFYPSDIAV EWESNGQPEN NYKTTPPVLD SDGSFFLYSK |
| | | LTVDKSRWQQ GNVFSCSVMH EALHNHYTQK SLSLSPGK |
| 144 | IgG1 K322A | EPKS CDKTHTCPPC PAPELLGGPS VFLFPPKPKD |
| | | TLMISRTPEV TCVVVDVSHE DPEVKENWYV DGVEVHNAKT |
| | | KPREEQYNST YRVVSVLTVL HQDWLNGKEY KCAVSNKALP |
| | | APIEKTISKA KGQPREPQVY TLPPSRDELT KNQVSLTCLV |
| | | KGFYPSDIAV EWESNGQPEN NYKTTPPVLD SDGSFFLYSK |
| | | LTVDKSRWQQ GNVFSCSVMH EALHNHYTQK SLSLSPGK |
| 145 | IgG4 | ES KYGPPCPSCP APEFLGGPSV FLFPPKPKDT |
| | | LMISRTPEVT CVVVDVSQED PEVQFNWYVD GVEVHNAKTK |
| | | PREEQFNSTY RVVSVLTVLH QDWLNGKEYK CKVSNKGLPS |
| | | SIEKTISKAK GQPREPQVYT LPPSQEEMTK NQVSLTCLVK |
| | | GFYPSDIAVE WESNGQPENN YKTTPPVLDS DGSFFLYSRS |
| | | TVDKSRWQEG NVFSCSVMHE ALHNHYTQKS LSLSLGK |
| 146 | IgG4 S228P | ES KYGPPCPPCP APEFLGGPSV FLFPPKPKDT |
| | | LMISRTPEVT CVVVDVSQED PEVQFNWYVD GVEVHNAKTK |
| | | PREEQFNSTY RVVSVLTVLH QDWLNGKEYK CKVSNKGLPS |
| | | SIEKTISKAK GQPREPQVYT LPPSQEEMTK NQVSLTCLVK |
| | | GFYPSDIAVE WESNGQPENN YKTTPPVLDS DGSFFLYSRL |
| | | TVDKSRWQEG NVFSCSVMHE ALHNHYTQKS LSLSLGK |
| 147 | Membrane proximal | LQSKATSGVTQG |
| | region of Siglec-9 ECD | |
| 148 | S9.32-W38T-hIgGI | SKLLTMQSSVTVQEGLCVHVPCSFSYPSHGTIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSlTCQVTFPGASVTTNKTVHLNVSYPPQNLTMTVF |
| | | QGDGTVSTVLGNGSSLSLPEGQSLRLVCAVDAVDSNPPARLSlSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 149 | S9.33-W38E-hIgG1 | SKLLTMQSSVTVQEGLCVHVPCSFSYPSHGEIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSESW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 150 | S9.34-W38S-hIgG1 | SKLLTMQSSVTVQEGLCVHVPCSFSYPSHGSIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 151 | S9.35-W38A-hIgG1 | SKLLTMQSSVTVQEGLCVHVPCSFSYPSHGAIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 152 | S9.36-W38R-hIgG1 | SKLLTMQSSVTVQEGLCVHVPCSFSYPSHGRIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 153 | S9.37-W38Q-hIgG1 | SKLLTMQSSVTVQEGLCVHVPCSFSYPSHGQIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 154 | S9.38-W38K-hIgG1 | SKLLTMQSSVTVQEGLCVHVPCSFSYPSHGKIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 155 | S9.39-W38S_Y40T- | SKLLTMQSSVTVQEGLCVHVPCSFSYPSHGSITPGPVVHGYWFRE |
| | hIgG1 | |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVE |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVELFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 156 | S9.41-L_ER_R (SEQ | SKLLTMQSSVTVQEGLCVHVPCSFSYPSHGERYPGPVVHGYWFRE |
| | ID NO: 263)-hIgG1 | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVE |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 157 | S9.42-D_SI_R (SEQ | SKDLTMQSSVTVQEGLCVHVPCSFSYPSHGSIYPGPVVHGYWFRE |
| | ID NO: 264)-hIgG1 | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 158 | S9.43-L KI_R (SEQ | SKLLTMQSSVTVQEGLCVHVPCSFSYPSHGKIYPGPVVHGYWFRE |
| | ID NO: 265)-hIgG1 | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 159 | S9.44-T_EI_E (SEQ | SKTLTMQSSVTVQEGLCVHVPCSFSYPSHGEIYPGPVVHGYWFRE |
| | ID NO: 266)-hIgG1 | |
| | | GANTDQDAPVATNNPARAVWEETRDRFHLLGDPHTKNCTLSIRDA |
| | | RRSDAGRYFFRMEKGSIKENYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 160 | S9.45-S_QI_R (SEQ | SKSLTMQSSVTVQEGLCVHVPCSFSYPSHGQIYPGPVVHGYWFRE |
| | ID NO: 267)-hIgG1 | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 161 | S9.47-R_SS_I_T (SEQ | SKLLTMQSSVTVQEGLCVHVPCSFSYPSHGWIYPGPVVHGYWFRE |
| | ID NO: 268)-hIgG1 | |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESRCPQNLTCSVPWACEQGTPPMISWIGTSSSPLDPSTTRSSVLT |
| | | LIPTPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 162 | S9.48-R DS I T | SKLLTMQSSVTVQEGLCVHVPCSFSYPSHGWIYPGPVVHGYWFRE |
| | (SEQ ID NO: 269)- | |
| | hIgG1 | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESRCPQNLTCSVPWACEQGTPPMISWIGTSDSPLDPSTTRSSVLT |
| | | LIPTPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 163 | S9.49-G_VT_Q_T | SKLLTMQSSVTVQEGLCVHVPCSFSYPSHGWIYPGPVVHGYWFRE |
| | (SEQ ID NO: 270)- | |
| | hIgG1 | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVTPLDPSTTRSSVLT |
| | | LQPTPQDHGTSLTCQVTFPGASVTTNKTVHLNVSYPPQNLTMTVF |
| | | QGDGTVSTVLGNGSSLSLPEGQSLRLVCAVDAVDSNPPARLSSSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 164 | S9.50-N_TT_I_Q | SKLLTMQSSVTVQEGLCVHVPCSFSYPSHGWIYPGPVVHGYWFRE |
| | (SEQ ID NO: 271)- | |
| | hIgG1 | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESNCPQNLTCSVPWACEQGTPPMISWIGTSTTPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 165 | S9.51-G_VK_I_E | SKLLTMQSSVTVQEGLCVHVPCSFSYPSHGWIYPGPVVHGYWFRE |
| | (SEQ ID NO: 272)- | |
| | hIgG1 | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVKPLDPSTTRSSVLT |
| | | LIPEPQDHGTSLTCQVTFPGASVTTNKTVHLNVSYPPQNLTMTVF |
| | | QGDGTVSTVLGNGSSLSLPEGQSLRLVCAVDAVDSNPPARLSESW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 166 | S9.52-R_SS_Q_T | SKLLTMQSSVTVQEGLCVHVPCSFSYPSHGWIYPGPVVHGYWFRE |
| | (SEQ ID NO: 273)- | |
| | hIgG1 | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESRCPQNLTCSVPWACEQGTPPMISWIGTSSSPLDPSTTRSSVLT |
| | | LQPTPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 167 | S9.53-G_SK_Q_E | SKLLTMQSSVTVQEGLCVHVPCSFSYPSHGWIYPGPVVHGYWFRE |
| | (SEQ ID NO: 274)- | |
| | hIgG1 | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSSKPLDPSTTRSSVLT |
| | | LQPEPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 168 | S9.36-W38R-hIgG1 | SKLLTMQSSVTVQEGLCVHVPCSFSYPSHGRIYPGPVVHGYWFRE |
| | NSLF | |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSSKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 169 | S9.37-W38Q-hIgG1 NSLF | SKLLTMQSSVTVQEGLCVHVPCSFSYPSHGQIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSSKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 170 | S9.38-W38K-hIgG1 NSLF | SKLLTMQSSVTVQEGLCVHVPCSFSYPSHGKIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VSEPKSCDKTHTCPPCPAPELLGGPSVFLFPPKPKDTLMISRTPE |
| | | VTCVVVDVSHEDPEVKENWYVDGVEVHNAKTKPREEQYNSTYRVV |
| | | SVLTVLHQDWLNGKEYKCKVSSKAFPAPIEKTISKAKGQPREPQV |
| | | YTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQ |
| | | KSLSLSPGK |
| 171 | S9.32 (SS)-W38T-hIgG1 | MGWSCIILFLVATATGVHSSKLLTMQSSVTVQEGLCVHVPCSFSY |
| | | PSHGTIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 172 | S9.33 (SS)-W38E-hIgG1 | MGWSCIILFLVATATGVHSSKLLTMQSSVTVQEGLCVHVPCSFSY |
| | | PSHGEIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 173 | S9.34 (SS)-W38S- | MGWSCIILFLVATATGVHSSKLLTMQSSVTVQEGLCVHVPCSFSY |
| | hIgG1 | |
| | | PSHGSIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 174 | S9.35 (SS)-W38A-hIgG1 | MGWSCIILFLVATATGVHSSKLLTMQSSVTVQEGLCVHVPCSFSY |
| | | PSHGAIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSERL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWINGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 175 | S9.36 (SS)-W38R-hIgG1 | MGWSCIILFLVATATGVHSSKLLTMQSSVTVQEGLCVHVPCSFSY |
| | | PSHGRIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 176 | S9.37 (SS)-W38Q-hIgG1 | MGWSCIILFLVATATGVHSSKLLTMQSSVTVQEGLCVHVPCSFSY |
| | | PSHGQIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKFNWYVDGVEV |
| | | HNAKTKPREEQYNSTYRVVSVLTVLHQDWINGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 177 | S9.38 (SS)-W38K-hIgG1 | MGWSCIILFLVATATGVHSSKLLTMQSSVTVQEGLCVHVPCSFSY |
| | | PSHGKIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 178 | S9.39 (SS)-W38S_Y40T-hIgG1 | MGWSCIILFLVATATGVHSSKLLTMQSSVTVQEGLCVHVPCSFSY |
| | | PSHGSITPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 179 | S9.41 (SS)-L_ER_R | MGWSCIILFLVATATGVHSSKLLTMQSSVTVQEGLCVHVPCSFSY |
| | (SEQ ID NO: 263)- | |
| | hIgG1 | PSHGERYPGPVVHGYWFREGANTDQDAPVATNNPARAVWEETRDR |
| | | FHLLGDPHTKNCTLSIRDARRSDAGRYFFRMEKGSIKRNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 180 | S9.42 (SS)-D_SI_R | MGWSCIILFLVATATGVHSSKDLTMQSSVTVQEGLCVHVPCSFSY |
| | (SEQ ID NO: 264)- | |
| | hIgG1 | PSHGSIYPGPVVHGYWFREGANTDQDAPVATNNPARAVWEETRDR |
| | | FHLLGDPHTKNCTLSIRDARRSDAGRYFFRMEKGSIKRNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 181 | S9.43 (SS)-L_KI_R | MGWSCIILFLVATATGVHSSKLLTMQSSVTVQEGLCVHVPCSFSY |
| | (SEQ ID NO: 265)- | |
| | hIgG1 | PSHGKIYPGPVVHGYWFREGANTDQDAPVATNNPARAVWEETRDR |
| | | FHLLGDPHTKNCTLSIRDARRSDAGRYFFRMEKGSIKRNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTEMISRTPEVTCVVVDVSHEDPEVKFNWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 182 | S9.44 (SS)-T_EI E | MGWSCIILFLVATATGVHSSKTLTMQSSVTVQEGLCVHVPCSFSY |
| | (SEQ ID NO: 266)- | |
| | hIgG1 | PSHGEIYPGPVVHGYWFREGANTDQDAPVATNNPARAVWEETRDR |
| | | FHLLGDPHTKNCTLSIRDARRSDAGRYFFRMEKGSIKENYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSURL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTAMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 183 | S9.45 (SS)-S_QI_R | MGWSCIILFLVATATGVHSSKSLTMQSSVTVQEGLCVHVPCSFSY |
| | (SEQ ID NO: 267)- | |
| | hIgG1 | PSHGQIYPGPVVHGYWFREGANTDQDAPVATNNPARAVWEETRDR |
| | | FHLLGDPHTKNCTLSIRDARRSDAGRYFFRMEKGSIKRNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTAMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 184 | S9.47 (SS)-R_SS_I_T | MGWSCIILFLVATATGVHSSKLLTMQSSVTVQEGLCVHVPCSFSY |
| | (SEQ ID NO: 268)- | |
| | hIgG1 | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESRCPQNLTCSVPWACEQGTPPMISW |
| | | IGTSSSPLDPSTTRSSVLTLIPTPQDHGTSLTCQVTFPGASVTTN |
| | | KTVHLNVSYPPQNLTMTVFQGDGTVSTVLGNGSSLSLPEGQSERL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTIMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 185 | S9.48 (SS)-R_DS_I_T | MGWSCIILFLVATATGVHSSKLLTMQSSVTVQEGLCVHVPCSFSY |
| | (SEQ ID NO: 269)- | |
| | hIgG1 | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESRCPQNLTCSVPWACEQGTPPMISW |
| | | IGTSDSPLDPSTTRSSVLTLIPTPQDHGTSLTCQVTFPGASVTTN |
| | | KTVHLNVSYPPQNLTMTVFQGDGTVSTVLGNGSSLSLPEGQSERL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTZMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 186 | S9.49 (SS)- | MGWSCIILFLVATATGVHSSKLLTMQSSVTVQEGLCVHVPCSFSY |
| | G_VT_Q_T (SEQ ID | |
| | NO: 270)-hIgG1 | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVTPLDPSTTRSSVLTLQPTPQDHGTSLTCQVTFPGASVTTN |
| | | KTVHLNVSYPPQNLTMTVFQGDGTVSTVLGNGSSLSLPEGQSLRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 187 | S9.50 (SS)-N_TT_I_Q | MGWSCIILFLVATATGVHSSKLLTMQSSVTVQEGLCVHVPCSFSY |
| | (SEQ ID NO: 271)- | |
| | hIgG1 | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESNCPQNLTCSVPWACEQGTPPMISW |
| | | IGTSTTPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTSMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 188 | S9.51 (SS)-G_VK_I_E | MGWSCIILFLVATATGVHSSKLLTMQSSVTVQEGLCVHVPCSFSY |
| | (SEQ ID NO: 272)- | |
| | hIgG1 | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVKPLDPSTTRSSVLTLIPEPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 189 | S9.52 (SS)-R_SS_Q_T | MGWSCIILFLVATATGVHSSKLLTMQSSVTVQEGLCVHVPCSFSY |
| | (SEQ ID NO: 273)- | |
| | hIgG1 | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESRCPQNLTCSVPWACEQGTPPMISW |
| | | IGTSSSPLDPSTTRSSVLTLQPTPQDHGTSLTCQVTFPGASVTTN |
| | | KTVHLNVSYPPQNLTMTVFQGDGTVSTVLGNGSSLSLPEGQSLRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 190 | S9.53 (SS)- | MGWSCIILFLVATATGVHSSKLLTMQSSVTVQEGLCVHVPCSFSY |
| | G_SK_Q_E (SEQ ID | |
| | NO: 274)-hIgG1 | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSSKPLDPSTTRSSVLTLQPEPQDHGTSLTCQVTFPGASVTTN |
| | | KTVHLNVSYPPQNLTMTVFQGDGTVSTVLGNGSSLSLPEGQSLRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 191 | S9.36 (SS)-W38R- | MGWSCIILFLVATATGVHSSKLLTMQSSVTVQEGLCVHVPCSFSY |
| | hIgG1 NSLF | |
| | | PSHGRIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSSKAFP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 192 | S9.37 (SS)-W38Q- | MGWSCIILFLVATATGVHSSKLLTMQSSVTVQEGLCVHVPCSFSY |
| | hIgG1 NSLF | |
| | | PSHGQIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWINGKEYKCKVSSKAFP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 193 | S9.38 (SS)-W38K- | MGWSCIILFLVATATGVHSSKLLTMQSSVTVQEGLCVHVPCSFSY |
| | hIgG1 NSLF | |
| | | PSHGKIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSERL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTAMISRTPEVTCVVVDVSHEDPEVKENWYVDGVEV |
| | | HNAKTKPREEQYNSTYRVVSVLTVLHQDWLNGKEYKCKVSSKAFP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 194 | S9.32 (EC)-W38T | SKLLTMQSSVTVQEGLCVHVPCSFSYPSHGTIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSESW |
| | | RGLTLCPSQPSNPGVLELPWVHLRDAAEFTCRAQNPLGSQQVYLN |
| | | VS |
| 195 | S9.33 (EC)-W38E | SKLLTMQSSVTVQEGLCVHVPCSFSYPSHGEIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 196 | S9.34 (EC)-W38S | SKLLTMQSSVTVQEGLCVHVPCSFSYPSHGSIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 197 | S9.35 (EC)-W38A | SKLLTMQSSVTVQEGLCVHVPCSFSYPSHGAIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 198 | S9.36 (EC)-W38R | SKLLTMQSSVTVQEGLCVHVPCSFSYPSHGRIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSSSW |
| | | RGLTLCPSQPSNPGVLELPWVHLRDAAEFTCRAQNPLGSQQVYLN |
| | | VS |
| 199 | S9.37 (EC)-W38Q | SKLLTMQSSVTVQEGLCVHVPCSFSYPSHGQIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 200 | S9.38 (EC)-W38K | SKLLTMQSSVTVQEGLCVHVPCSFSYPSHGKIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 201 | S9.39 (EC)- | SKLLTMQSSVTVQEGLCVHVPCSFSYPSHGSITPGPVVHGYWFRE |
| | W38S_Y40T | |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 202 | S9.41 (EC)-L_ER_R | SKLLTMQSSVTVQEGLCVHVPCSFSYPSHGERYPGPVVHGYWFRE |
| | (SEQ ID NO: 263) | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSSSW |
| | | RGLTLCPSQPSNPGVLELPWVHLRDAAEFTCRAQNPLGSQQVYLN |
| | | VS |
| 203 | S9.42 (EC)-D_SI_R | SKDLTMQSSVTVQEGLCVHVPCSFSYPSHGSIYPGPVVHGYWFRE |
| | (SEQ ID NO: 264) | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSESW |
| | | RGLTLCPSQPSNPGVLELPWVHLRDAAEFTCRAQNPLGSQQVYLN |
| | | VS |
| 204 | S9.43 (EC)-L_KI_R | SKLLTMQSSVTVQEGLCVHVPCSFSYPSHGKIYPGPVVHGYWFRE |
| | (SEQ ID NO: 265) | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVE |
| | | QGDGTVSTVLGNGSSLSLPEGQSLRLVCAVDAVDSNPPARLSESW |
| | | RGLTLCPSQPSNPGVLELPWVHLRDAAEFTCRAQNPLGSQQVYLN |
| | | VS |
| 205 | S9.44 (EC)-T_EI_E | SKTLTMQSSVTVQEGLCVHVPCSFSYPSHGEIYPGPVVHGYWFRE |
| | (SEQ ID NO: 266) | |
| | | GANTDQDAPVATNNPARAVWEETRDRFHLLGDPHTKNCTLSIRDA |
| | | RRSDAGRYFFRMEKGSIKENYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 206 | S9.45 (EC)-S_QI_R | SKSLTMQSSVTVQEGLCVHVPCSFSYPSHGQIYPGPVVHGYWFRE |
| | (SEQ ID NO: 267) | |
| | | GANTDQDAPVATNNPARAVWEETRDRFHLLGDPHTKNCTLSIRDA |
| | | RRSDAGRYFFRMEKGSIKRNYKHHRLSVNVTALTHRPNILIPGTL |
| | | ESGCPQNLTCSVPWACEQGTPPMISWIGTSVSPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 207 | S9.47 (EC)-R_SS_I_T | SKLLTMQSSVTVQEGLCVHVPCSFSYPSHGWIYPGPVVHGYWFRE |
| | (SEQ ID NO: 268) | |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESRCPQNLTCSVPWACEQGTPPMISWIGTSSSPLDPSTTRSSVLT |
| | | LIPTPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 208 | S9.48 (EC)-R_DS I T | SKLLTMQSSVTVQEGLCVHVPCSFSYPSHGWIYPGPVVHGYWFRE |
| | (SEQ ID NO: 269) | |
| | | GANTDQDAPVATNNPARAVWEETRDREHLLGDPHTKNCTLSIRDA |
| | | RRSDAGRYFFRMEKGSIKWNYKHHRLSVNVTALTHRPNILIPGTL |
| | | ESRCPQNLTCSVPWACEQGTPPMISWIGTSDSPLDPSTTRSSVLT |
| | | LIPTPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 209 | S9.49 (EC)- | SKLLTMQSSVTVQEGLCVHVPCSFSYPSHGWIYPGPVVHGYWFRE |
| | G_VT_Q_T (SEQ ID | |
| | NO: 270) | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVTPLDPSTTRSSVLT |
| | | LQPTPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 210 | S9.50 (EC)-N_TT_I_Q | SKLLTMQSSVTVQEGLCVHVPCSFSYPSHGWIYPGPVVHGYWFRE |
| | (SEQ ID NO: 271) | |
| | | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESNCPQNLTCSVPWACEQGTPPMISWIGTSTTPLDPSTTRSSVLT |
| | | LIPQPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 211 | S9.51 (EC)- | SKLLTMQSSVTVQEGLCVHVPCSFSYPSHGWIYPGPVVHGYWFRE |
| | G_VK_I_E (SEQ ID | |
| | NO: 272) | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSVKPLDPSTTRSSVLT |
| | | LIPEPQDHGTSLTCQVTFPGASVTTNKTVHLNVSYPPQNLTMTVF |
| | | QGDGTVSTVLGNGSSLSLPEGQSLRLVCAVDAVDSNPPARLSSSW |
| | | RGLTLCPSQPSNPGVLELPWVHLRDAAEFTCRAQNPLGSQQVYLN |
| | | VS |
| 212 | S9.52 (EC)- | SKLLTMQSSVTVQEGLCVHVPCSFSYPSHGWIYPGPVVHGYWFRE |
| | R_SS_Q_T (SEQ ID | |
| | NO: 273) | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESRCPQNLTCSVPWACEQGTPPMISWIGTSSSPLDPSTTRSSVLT |
| | | LQPTPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 213 | S9.53 (EC)- | SKLLTMQSSVTVQEGLCVHVPCSFSYPSHGWIYPGPVVHGYWFRE |
| | G_SK_Q_E (SEQ ID | |
| | NO: 274) | GANTDQDAPVATNNPARAVWEETRDRFHLLGDPHTKNCTLSIRDA |
| | | RRSDAGRYFFRMEKGSIKWNYKHHRLSVNVTALTHRPNILIPGTL |
| | | ESGCPQNLTCSVPWACEQGTPPMISWIGTSSKPLDPSTTRSSVLT |
| | | LQPEPQDHGTSLTCQVTFPGASVTTNKTVHLNVSYPPQNLTMTVF |
| | | QGDGTVSTVLGNGSSLSLPEGQSLRLVCAVDAVDSNPPARLSLSW |
| | | RGLTLCPSQPSNPGVLELPWVHLRDAAEFTCRAQNPLGSQQVYLN |
| | | VS |
| 214 | S9.32 (IgV)-W38T | SKLLTMQSSVTVQEGLCVHVPCSFSYPSHGTIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 215 | S9.33 (IgV)-W38E | SKLLTMQSSVTVQEGLCVHVPCSFSYPSHGEIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 216 | S9.34 (IgV)-W38S | SKLLTMQSSVTVQEGLCVHVPCSFSYPSHGSIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 217 | S9.35 (IgV)-W38A | SKLLTMQSSVTVQEGLCVHVPCSFSYPSHGAIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 218 | S9.36 (IgV)-W38R | SKLLTMQSSVTVQEGLCVHVPCSESYPSHGRIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 219 | S9.37 (IgV)-W38Q | SKLLTMQSSVTVQEGLCVHVPCSFSYPSHGQIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 220 | S9.38 (IgV)-W38K | SKLLTMQSSVTVQEGLCVHVPCSFSYPSHGKIYPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 221 | S9.39 (IgV)-W38S Y40T | SKLLTMQSSVTVQEGLCVHVPCSFSYPSHGSITPGPVVHGYWFRE |
| | | GANTDQDAPVATNNPARAVWEETRDRFHLLGDPHTKNCTLSIRDA |
| | | RRSDAGRYFFRMEKGSIKWNYKHHRLSVNVT |
| 222 | S9.41 (IgV)-L_ER_R | SKLLTMQSSVTVQEGLCVHVPCSFSYPSHGERYPGPVVHGYWFRE |
| | (SEQ ID NO: 263) | |
| | | GANTDQDAPVATNNPARAVWEETRDREHLLGDPHTKNCTLSIRDA |
| | | RRSDAGRYFFRMEKGSIKRNYKHHRLSVNVT |
| 223 | S9.42 (IgV)-D_SI_R | SKDLTMQSSVTVQEGLCVHVPCSFSYPSHGSIYPGPVVHGYWFRE |
| | (SEQ ID NO: 264) | |
| | | GANTDQDAPVATNNPARAVWEETRDRFHLLGDPHTKNCTLSIRDA |
| | | RRSDAGRYFFRMEKGSIKRNYKHHRLSVNVT |
| 224 | S9.43 (IgV)-L_KI_R | SKLLTMQSSVTVQEGLCVHVPCSFSYPSHGKIYPGPVVHGYWFRE |
| | (SEQ ID NO: 265) | |
| | | GANTDQDAPVATNNPARAVWEETRDRFHLLGDPHTKNCTLSIRDA |
| | | RRSDAGRYFFRMEKGSIKRNYKHHRLSVNVT |
| 225 | S9.44 (IgV)-T_EI_E | SKTLTMQSSVTVQEGLCVHVPCSFSYPSHGEIYPGPVVHGYWFRE |
| | (SEQ ID NO: 266) | |
| | | GANTDQDAPVATNNPARAVWEETRDRFHLLGDPHTKNCTLSIRDA |
| | | RRSDAGRYFFRMEKGSIKENYKHHRLSVNVT |
| 226 | S9.45 (IgV)-S_QI_R | SKSLTMQSSVTVQEGLCVHVPCSFSYPSHGQIYPGPVVHGYWFRE |
| | (SEQ ID NO: 267) | |
| | | GANTDQDAPVATNNPARAVWEETRDRFHLLGDPHTKNCTLSIRDA |
| | | RRSDAGRYFFRMEKGSIKRNYKHHRLSVNVT |
| 227 | S9.1-hIgGI NSLF (no | S KLLTMQSSVT VQEGLCVHVP CSFSYPSHGW IYPGPVVHGY |
| | SS) | |
| | | WFREGANTDQ DAPVATNNPA RAVWEETRDR FHLLGDPHTK |
| | | NCTLSIRDAR RSDAGRYFFR MEKGSIKWNY KHHRLSVNVT |
| | | ALTHRPNILI PGTLESGCPQ NLTCSVPWAC EQGTPPMISW |
| | | IGTSVSPLDP STTRSSVLTL IPQPQDHGTS LTCQVTFPGA |
| | | SVTTNKTVHL NVSYPPQNLT MTVFQGDGTV STVLGNGSSL |
| | | SLPEGQSLRL VCAVDAVDSN PPARLSLSWR GLTLCPSQPS |
| | | NPGVLELPWV HLRDAAEFTC RAQNPLGSQQ VYLNVSEPKS |
| | | CDKTHTCPPC PAPELLGGPS VFLFPPKPKD TLMISRTPEV |
| | | TCVVVDVSHE DPEVKENWYV DGVEVHNAKT KPREEQYNST |
| | | YRVVSVLTVL HQDWLNGKEY KCKVSSKAFP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 228 | S9.1-hIgG1 NSLF YTE | MGWSCIILFLVATATGVHSSKLLTMQSSVTVQEGLCVHVPCSFSY |
| | | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTYITREPEVTCVVVDVSHEDPEVKENWYVDGVEV |
| | | HNAKTKPREEQYNSTYRVVSVLTVLHQDWLNGKEYKCKVSSKAFP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 229 | S9.1-hIgGI NSLF LS | MGWSCIILFLVATATGVHSSKLLTMQSSVTVQEGLCVHVPCSFSY |
| | | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWINGKEYKCKVSSKAFP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVLHEALHSHYTQKSLSLSPGK |
| 230 | S9.1-hIgGI NSLF LV5-112 | MGWSCIILFLVATATGVHSSKLLTMQSSVTVQEGLCVHVPCSFSY |
| | | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTLMISRTPEVTCVVVDVSHEDPEVKENWYVDGVEV |
| | | HNAKTKPREEQYNSTYRVVSVLTVLHQDWLNGKEYKCKVSSKAFP |
| | | APIEKTISKAKGQPREPQVYTLPPSRDELTKNQVSLTCLVKGFYP |
| | | SDIAVEWESNGGCALYPTNCGGGQPENNYKTTPPVLDSDGSFFLY |
| | | SKLTVDKSRWQQGNVFSCSVMHEALHNHYTQKSLSLSPGK |
| 231 | S9.1-hIgGI YTE | MGWSCIILFLVATATGVHSSKLLTMQSSVTVQEGLCVHVPCSFSY |
| | | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSERL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTSYITREPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVMHEALHNHYTQKSLSLSPGK |
| 232 | S9.1-hIgG1 LS | MGWSCIILFLVATATGVHSSKLLTMQSSVTVQEGLCVHVPCSFSY |
| | | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSSRL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTAMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGQPENNYKTTPPVLDSDGSFFLYSKLTVDKSRWQQ |
| | | GNVFSCSVLHEALHSHYTQKSLSLSPGK |
| 233 | S9.1-hIgG1 LV5-112 | MGWSCIILFLVATATGVHSSKLLTMQSSVTVQEGLCVHVPCSFSY |
| | | PSHGWIYPGPVVHGYWFREGANTDQDAPVATNNPARAVWEETRDR |
| | | FHLLGDPHTKNCTLSIRDARRSDAGRYFFRMEKGSIKWNYKHHRL |
| | | SVNVTALTHRPNILIPGTLESGCPQNLTCSVPWACEQGTPPMISW |
| | | IGTSVSPLDPSTTRSSVLTLIPQPQDHGTSLTCQVTFPGASVTTN |
| | | KTVHLNVSYPPQNLTMTVFQGDGTVSTVLGNGSSLSLPEGQSARL |
| | | VCAVDAVDSNPPARLSLSWRGLTLCPSQPSNPGVLELPWVHLRDA |
| | | AEFTCRAQNPLGSQQVYLNVSEPKSCDKTHTCPPCPAPELLGGPS |
| | | VFLFPPKPKDTAMISRTPEVTCVVVDVSHEDPEVKENWYVDGVEV |
| | | HNAKTKPREEQYNSTYRVVSVLTVLHQDWLNGKEYKCKVSNKALP |
| | | APIEKTISKAKGQPREPQVYTLPPSRDELTKNQVSLTCLVKGFYP |
| | | SDIAVEWESNGGCALYPTNCGGGQPENNYKTTPPVLDSDGSFFLY |
| | | SKLTVDKSRWQQGNVFSCSVMHEALHNHYTQKSLSLSPGK |
| 234 | hIgG1 NSLF YTE | EPKSCDKTHTCPPCPAPELLGGPSVELFPPKPKDTLYITREPEVT |
| | | CVVVDVSHEDPEVKFNWYVDGVEVHNAKTKPREEQYNSTYRVVSV |
| | | LTVLHQDWLNGKEYKCKVSSKAFPAPIEKTISKAKGQPREPQVYT |
| | | LPPSRDELTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPGK |
| 235 | hIgG1 NSLF LS | EPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSV |
| | | LTVLHQDWLNGKEYKCKVSSKAFPAPIEKTISKAKGQPREPQVYT |
| | | LPPSRDELTKNQVSLTCLVKGFYPSDIAVEWESNGQPENNYKTTP |
| | | PVLDSDGSFFLYSKLTVDKSRWQQGNVFSCSVLHEALHSHYTQKS |
| | | LSLSPGK |
| 236 | hIgG1 NSLF LV5-112 | EPKSCDKTHTCPPCPAPELLGGPSVELFPPKPKDTLMISRTPEVT |
| | | CVVVDVSHEDPEVKFNWYVDGVEVHNAKTKPREEQYNSTYRVVSV |
| | | LTVLHQDWLNGKEYKCKVSSKAFPAPIEKTISKAKGQPREPQVYT |
| | | LPPSRDELTKNQVSLTCLVKGFYPSDIAVEWESNGGCALYPTNCG |
| | | GGQPENNYKTTPPVLDSDGSFFLYSKLTVDKSRWQQGNVESCSVM |
| | | HEALHNHYTQKSLSLSPGK |
| 237 | hIgG1 YTE | EPKSCDKTHTCPPCPAPELLGGPSVFLFPPKPKDTLYITREPEVT |
| | | CVVVDVSHEDPEVKFNWYVDGVEVHNAKTKPREEQYNSTYRVVSV |
| | | LTVLHQDWLNGKEYKCKVSNKALPAPIEKTISKAKGQPREPQVYT |
| | | LPPSRDELTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPGK |
| 238 | hIgG1 LS | EPKSCDKTHTCPPCPAPELLGGPSVELFPPKPKDTLMISRTPEVT |
| | | CVVVDVSHEDPEVKFNWYVDGVEVHNAKTKPREEQYNSTYRVVSV |
| | | LTVLHQDWLNGKEYKCKVSNKALPAPIEKTISKAKGQPREPQVYT |
| | | LPPSRDELTKNQVSLTCLVKGFYPSDIAVEWESNGQPENNYKTTP |
| | | PVLDSDGSFFLYSKLTVDKSRWQQGNVFSCSVLHEALHSHYTQKS |
| | | LSLSPGK |
| 239 | hIgG1 LV5-112 | EPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSV |
| | | LTVLHQDWLNGKEYKCKVSNKALPAPIEKTISKAKGQPREPQVYT |
| | | LPPSRDELTKNQVSLTCLVKGFYPSDIAVEWESNGGCALYPTNCG |
| | | GGQPENNYKTTPPVLDSDGSFFLYSKLTVDKSRWQQGNVFSCSVM |
| | | HEALHNHYTQKSLSLSPGK |
| 240 | 2D4 HVR-L1 | RASQEISGYLG |
| 241 | 2D4 HVR-L2 | STSTLDS |
| 242 | 2D4 HVR-L3 | LQYASYPPT |
| 243 | 2D4 HVR-H1 | FTFSNYAMS |
| 244 | 2D4 HVR-H2 | VATINNGGSYTYYS |
| 245 | 2D4 HVR-H3 | VRRDYGTSDEDY |
| 246 | 2D4 light chain variable | DIQMTQSPSSLSASLGERVSLTCRASQEISGYLGWLQQKPDGTIK |
| | region | |
| | | RLIFSTSTLDSGVPKRFSGSRSGSDYSLTISSLESEDFADYYCLQ |
| | | YASYPPTFGGGTKLEIK |
| 247 | 2D4 heavy chain | EVKLVESGGALVKPGGSLKLSCAASGFTFSNYAMSWVRQTPEKRL |
| | variable region | |
| | | EWVATINNGGSYTYYSDSVKGRFAISRDNAKNTLYLQMSNLRSED |
| | | TALYYCVRRDYGTSDFDYWGQGTTLTVSS |
| 248 | | DIEG |
| 249 | | SIET |
| 250 | | SIEP |
| 251 | | DIEP |
| 252 | | YQES |
| 253 | | THET |
| 254 | | WIYP |
| 255 | | QTDS |
| 256 | | GWIYP |
| 257 | | SQTDS |
| 258 | | VHGQIDSD |
| 259 | | VHSQIDSD |
| 260 | | VDSQIDSD |
| 261 | | SDSQIDSD |
| 262 | | VDGQIDSD |
| 263 | | LERR |
| 264 | | DSIR |
| 265 | | LKIR |
| 266 | | TEIE |
| 267 | | SQIR |
| 268 | | RSSIT |
| 269 | | RDSIT |
| 270 | | GVTQT |
| 271 | | NTTIQ |
| 272 | | GVKIE |
| 273 | | RSSQT |
| 274 | | GSKQE |

What is claimed is:

1. A method of treating cancer in a subject in need thereof, comprising administering to the subject a Siglec-9 extracellular domain (ECD) fusion polypeptide, wherein an elevated expression level of CD163 and/or Siglec-9 has been detected in a tumor sample from the subject.

2. A method of predicting a response to treatment with a Siglec-9 extracellular domain (ECD) fusion polypeptide in a subject having cancer, comprising determining an expression level of CD163 and/or Siglec-9 in a tumor sample from the subject, wherein an elevated expression level of CD163 and/or Siglec-9 predicts response to treatment with the Siglec-9 ECD fusion polypeptide.

3. A method of selecting a subject with cancer for treatment with a Siglec-9 extracellular domain (ECD) fusion polypeptide, comprising selecting the subject for such treatment if a tumor sample from the subject has been determined to have an elevated expression level of CD163 and/or Siglec-9.

4. The method of claim 2 or 3, further comprising administering a Siglec-9 ECD fusion polypeptide to the subject.

5. A method of treating cancer in a subject in need thereof, comprising determining that a tumor sample from the subject has an elevated expression level of CD163 and/or Siglec-9, and administering a Siglec-9 extracellular domain (ECD) fusion polypeptide to the subject.

6. The method of any one of claims 1-5, wherein the tumor sample from the subject has elevated expression levels of CD163 and Siglec-9.

7. The method of any one of claims 1-6, wherein the tumor sample from the subject has an elevated expression level of CD68.

8. The method of any one of claims 1-7, wherein the tumor sample from the subject has an elevated level of sialic acid.

9. The method of any one of claims 1-8, wherein the tumor sample from the subject is obtained from a tumor biopsy.

10. The method of any one of claims 1-9, wherein the expression level of CD163 and/or Siglec-9, and/or optionally CD68, and/or optionally the level of sialic acid, is detected by immunohistochemistry (IHC).

11. The method of claim 10, wherein the expression level of CD163, Siglec-9, or CD68 or the level of sialic acid is determined to be elevated if IHC detects 20-100 stained cells per high power field, or greater than 100 stained cells per high power field, or if the IHC score on a scale of 0-3 is IHC 2 or IHC 3, for CD163, Siglec-9, CD68 or sialic acid.

12. The method of claim 10 or 11, wherein the expression level of CD163, Siglec-9, or CD68 or the level of sialic acid is determined to be elevated if the IHC score is IHC 2 or IHC 3.

13. The method of claim 10 or 11, wherein the expression levels of CD163 and Siglec-9 are determined to be elevated, and the IHC scores for CD163 and Siglec-9 are selected from: CD163 IHC 3 and Siglec-9 IHC 3; CD163 IHC 2 and Siglec-9 IHC 3; CD163 IHC 3 and Siglec-9 IHC 2: or CD163 IHC 2 and Siglec-9 IHC 2.

14. The method of claim 10 or 11, wherein the expression levels of CD163, Siglec-9 and CD68 are determined to be elevated, and the IHC scores for CD163, Siglec-9, and CD68 are selected from: CD163 IHC 3, Siglec-9 IHC 3, and CD68 IHC 3; CD163 IHC 2, Siglec-9 IHC 3, and CD68 IHC 3: CD163 IHC 3, Siglec-9 IHC 2, and CD68 IHC 3: CD163 IHC 2, Siglec-9 IHC 2, and CD68 IHC 3: CD163 IHC 3, Siglec-9 IHC 3, and CD68 IHC 2; CD163 IHC 2, Siglec-9 IHC 3, and CD68 IHC 2; CD163 IHC 3, Siglec-9 IHC 2, and CD68 IHC 2; or CD163 IHC 2, Siglec-9 IHC 2, and CD68 IHC 2.

15. The method of claim 10 or 11, wherein the expression levels of CD163 and Siglec-9 and the level of sialic acid are determined to be elevated, and the IHC scores for CD163, Siglec-9, and sialic acid are selected from: CD163 IHC 3, Siglec-9 IHC 3, and sialic acid IHC 3: CD163 IHC 2, Siglec-9 IHC 3, and sialic acid IHC 3; CD163 IHC 3, Siglec-9 IHC 2, and sialic acid IHC 3; CD163 IHC 2, Siglec-9 IHC 2, and sialic acid IHC 3; CD163 IHC 3, Siglec-9 IHC 3, and sialic acid IHC 2; CD163 IHC 2, Siglec-9 IHC 3, and sialic acid IHC 2; CD163 IHC 3, Siglec-9 IHC 2, and sialic acid IHC 2; or CD163 IHC 2, Siglec-9 IHC 2, and sialic acid IHC 2.

16. The method of claim 10 or 11, wherein the expression levels of CD163, Siglec-9 and CD68 and the level of sialic acid are determined to be elevated, and the IHC scores for CD163, Siglec-9, CD68, and sialic acid are selected from: CD163 IHC 3, Siglec-9 IHC 3, CD68 IHC 3, and sialic acid IHC 3; CD163 IHC 2, Siglec-9 IHC 3, CD68 IHC 3, and sialic acid IHC 3; CD163 IHC 3, Siglec-9 IHC 2, CD68 IHC 3, and sialic acid IHC 3; CD163 IHC 2, Siglec-9 IHC 2, CD68 IHC 3, and sialic acid IHC 3; CD163 IHC 3, Siglec-9 IHC 3, CD68 IHC 2, and sialic acid IHC 3; CD163 IHC 2, Siglec-9 IHC 3, CD68 IHC 2, and sialic acid IHC 3; CD163 IHC 3, Siglec-9 IHC 2, CD68 IHC 2, and sialic acid IHC 3; CD163 IHC 2, Siglec-9 IHC 2, CD68 IHC 2, and sialic acid IHC 3; CD163 IHC 3, Siglec-9 IHC 3, CD68 IHC 3, and sialic acid IHC 2; CD163 IHC 2, Siglec-9 IHC 3, CD68 IHC 3, and sialic acid IHC 2; CD163 IHC 3, Siglec-9 IHC 2, CD68 IHC 3, and sialic acid IHC 2: CD163 IHC 2, Siglec-9 IHC 2, CD68 IHC 3, and sialic acid IHC 2; CD163 IHC 3, Siglec-9 IHC 3, CD68 IHC 2, and sialic acid IHC 2: CD163 IHC 2, Siglec-9 IHC 3, CD68 IHC 2, and sialic acid IHC 2: CD163 IHC 3, Siglec-9 IHC 2, CD68 IHC 2, or sialic acid IHC 2; CD163 IHC 2, Siglec-9 IHC 2, CD68 IHC 2, and sialic acid IHC 2.

17. The method of any one of claims 10-16, wherein IHC for Siglec-9 is conducted with an anti-Siglec-9 antibody comprising an HVR-L1 comprising the amino acid sequence of SEQ ID NO: 240, an HVR-L2 comprising the amino acid sequence of SEQ ID NO: 241, an HVR-L3 comprising the amino acid sequence of SEQ ID NO: 242, an HVR-H1 comprising the amino acid sequence of SEQ ID NO: 243, an HVR-H2 comprising the amino acid sequence of SEQ ID NO: 244, and an HVR-H3 comprising the amino acid sequence of SEQ ID NO: 245.

18. The method of any one of claims 10-17, wherein IHC for Siglec-9 is conducted with an anti-Siglec-9 antibody comprising a heavy chain variable domain comprising the amino acid sequence of SEQ ID NO: 247 and a light chain variable domain comprising the amino acid sequence of SEQ ID NO: 246, optionally wherein the antibody is a murine IgG2A antibody.

19. The method of any one of claims 1-18, wherein the cancer is a solid tumor associated with a tumor microenvironment comprising mycloid cells.

20. The method of any one of claims 1-19, wherein the cancer is selected from renal cell carcinoma, sarcoma, pancreatic cancer, glioblastoma, ovarian cancer, colorectal cancer, lung cancer, melanoma, head and neck cancer, breast cancer, and gastric cancer.

21. The method of any one of claims 1-20, wherein the cancer is metastatic.

22. The method of any one of claims 1-21, wherein the Siglec-9 ECD fusion polypeptide comprises a Siglec-9 IgV domain comprising an amino acid sequence selected from any one of SEQ ID NOs: 109-137 and 214-226.

23. The method of claim 22, wherein the polypeptide comprises a Siglec-9 ECD comprising the Siglec-9 IgV domain, a C2 type 1 (C2T1) domain, and a C2 type 2 (C2T2) domain.

24. The method of claim 22 or claim 23, wherein the polypeptide comprises an amino acid sequence selected from any one of SEQ ID NOs: 79-107 and 194-206.

25. The method of any one of claims 22-24, wherein the polypeptide further comprises an Fc domain.

26. The method of claim 25, wherein the Fc domain is located at the C-terminus of the polypeptide.

27. The method of claim 25 or claim 26, wherein the Fc domain has a human IgG1 isotype.

28. The method of claim 27, wherein the Fc domain has a human IgG1 isotype that has:
a) reduced binding to FcγRIII;
b) reduced antibody-dependent cellular cytotoxicity (ADCC) and/or reduced complement binding activity;
c) increased binding to FcγRIIa; or
d) any combination of a), b), and/or c).
relative to the IgG1 polypeptide of SEQ ID No: 142.

29. The method of claim 25 or 26, wherein the Fc domain comprises an amino acid sequence selected from any one of SEQ ID NOs: 142-144 and 234-239.

30. The method of claim 29, wherein the Fc domain comprises the amino acid sequence of SEQ ID NO: 142 or 143.

31. The method of claim 25 or 26, wherein the polypeptide comprises an amino acid sequence selected from any one of SEQ ID NOs: 11-39, 148-160, and 168-170.

32. The method of claim 31, wherein the polypeptide comprises an amino acid sequence selected from any one of SEQ ID NOs: 49-77, 171-183, and 191-193.

33. The method of any one of claims 1-21, wherein the Siglec-9 ECD fusion polypeptide comprising an amino acid sequence selected from any one of SEQ ID NOs: 49-77 and 171-193, lacking its signal peptide.

34. The method of claim 25 or claim 26, wherein the Fc domain has a human IgG4 isotype.

35. The method of claim 34, wherein the Fc domain comprises the amino acid sequence of SEQ ID NO: 145 or 146.

36. The method of any one of claims 1-21, wherein the Siglec-9 ECD fusion polypeptide comprises the amino acid sequence of SEQ ID NO: 138.

37. The method of claim 36, wherein the polypeptide further comprises an Fc domain.

38. The method of claim 37, wherein the Fc domain is located at the C-terminus of the polypeptide.

39. The method of claim 38, wherein the Fc domain has a human IgG1 isotype.

40. The method of claim 39, comprising a linker sequence.

41. The method of claim 37 or 38, wherein the Fc domain comprises an amino acid sequence selected from any one of SEQ ID NOs: 142-144.

42. The method of claim 41, wherein the Fc domain comprises the amino acid sequence of SEQ ID NO: 142 or 143.

43. The method of claim 39, wherein the polypeptide comprises the amino acid sequence of SEQ ID NO: 139.

44. The method of claim 37, wherein the Fc domain has a human IgG4 isotype.

45. The method of claim 44, wherein the Fc domain comprises the amino acid sequence of SEQ ID NO: 145 or 146.

46. The method of any one of claims 1-21, wherein the Siglec-9 ECD fusion polypeptide comprises SEQ ID NO: 78 joined at its C-terminus to an Fc domain.

47. The method of claim 46, wherein the Fc domain has a human IgG1 or IgG4 isotype.

48. The method of claim 47, wherein the Fc domain has a human IgG1 isotype that has:
a) reduced binding to FcγRIII;
b) reduced antibody-dependent cellular cytotoxicity (ADCC) and/or reduced complement binding activity;
c) increased binding to FcγRIIa; or
d) any combination of a), b), and/or c).
relative to the IgG1 polypeptide of SEQ ID No: 142.

49. The method of claim 47 or 48, wherein the Fc domain has a human IgG1 isotype and comprises an amino acid sequence selected from any one of SEQ ID NOs: 142-144 and 234-239.

50. The method of claim 47, wherein the Fc domain comprises the amino acid sequence of SEQ ID NO: 142.

51. The method of claim 47, wherein the polypeptide comprises the amino acid sequence of SEQ ID NO:10.

52. The method of claim 47 or 48, wherein the Fc domain comprises the amino acid sequence of SEQ ID NO: 143.

53. The method of claim 47, wherein the polypeptide comprises the amino acid sequence of SEQ ID NO: 227.

54. The method of claim 47, wherein the Fc domain has a human IgG4 isotype and comprises the amino acid sequence of SEQ ID NO: 145 or 146.

55. The method of claim 46, wherein the polypeptide comprises an amino acid sequence selected from any one of SEQ ID NOs: 45-48 and 228-233, lacking its associated signal peptide.

56. The method of claim 55, wherein the polypeptide comprises an amino acid sequence selected from any one of SEQ ID NOs: 45-48 and 228-233.

57. The method of claim 55, wherein the polypeptide comprises the amino acid sequence of SEQ ID NO: 45, lacking its associated signal peptide.

58. The method of claim 57, wherein the polypeptide comprises the amino acid sequence of SEQ ID NO: 45.

59. The method of claim 55, wherein the polypeptide comprises the amino acid sequence of SEQ ID NO: 48, lacking its associated signal peptide.

60. The method of any one of claims 1-21, wherein the Siglec-9 ECD fusion polypeptide comprises an amino acid sequence selected from any one of SEQ ID NOs: 207-213 and an Fc domain located at the C-terminus of the polypeptide.

61. The method of claim 60, wherein the Fc domain has a human IgG1 or IgG4 isotype.

62. The method of claim 61, wherein the Fc domain has a human IgG1 isotype that has:
a) reduced binding to FecγRIII;
b) reduced antibody-dependent cellular cytotoxicity (ADCC) and/or reduced complement binding activity;
c) increased binding to FcγRIIa; or
d) any combination of a), b), and/or c).
relative to the IgG1 polypeptide of SEQ ID No: 142.

63. The method of claim 61 or 62, wherein the Fc domain has a human IgG1 isotype and comprises an amino acid sequence selected from any one of SEQ ID NOs: 142-144 and 234-239.

64. The method of claim 60, comprising an amino acid sequence selected from any one of SEQ ID NOs: 161-167.

65. The method of claim 61, comprising an amino acid sequence selected from any one of SEQ ID NOs: 184-190, lacking its associated signal peptide.

66. The method of claim 65, comprising an amino acid sequence selected from any one of SEQ ID NOs: 184-190.

67. The method of claim 61, wherein the Fc domain has a human IgG4 isotype and comprises the amino acid sequence of SEQ ID NO: 145 or 146.

68. The method of any one of claims 1 or 4-67, further comprising administering an antagonist of PD-1 or PD-L1, optionally wherein the antagonist of PD-1 or PD-L1 is an antibody that binds to PD-1 or PD-L1, respectively.

69. The method of any one of claims 1 or 4-68, further comprising administering a chemotherapeutic agent.


SEQUENCE LISTING (274 sequences)

SEQ ID NO: 1 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 2 - protein, 444 aa, Homo sapiens
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 3 - protein, 5 aa, synthetic construct
ALTHR

SEQ ID NO: 4 - protein, 6 aa, synthetic construct
LNVSYP

SEQ ID NO: 5 - protein, 6 aa, synthetic construct
LQYASL

SEQ ID NO: 6 - protein, 6 aa, synthetic construct
TEYSEI

SEQ ID NO: 7 - protein, 121 aa, Homo sapiens
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 8 - protein, 8 aa, synthetic construct
VDSQTDSD

SEQ ID NO: 9 - protein, 8 aa, synthetic construct
SHGWIYPG

SEQ ID NO: 10 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 549 aa, synthetic construct
SKLLTMQSSVTVQEGLCVHVPCSFSYPSHGDIEGGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 549 aa, synthetic construct
SKLLTMQSSVTVQEGLCVHVPCSFSYPSHGSIETGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 549 aa, synthetic construct
SKLLTMQSSVTVQEGLCVHVPCSFSYPSHGSIEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 549 aa, synthetic construct
SKLLTMQSSVTVQEGLCVHVPCSFSYPSHGDIEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 549 aa, synthetic construct
SKLLTMQSSVTVQEGLCVHVPCSFSYPSHGYQESGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 549 aa, synthetic construct
SKLLTMQSSVTVQEGLCVHVPCSFSYPSHGTHETGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 549 aa, synthetic construct
SKLLTMQSSVTVQEGTCVSVPCSFSYPSHGWIYPGPVVHGYWFREGANTDQDAPVATNNPARAVWEETRDRFYLEGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 549 aa, synthetic construct
SKLLTMQSSVTVQEGTCVTVPCSFSYPSHGWIYPGPVVHGYWFREGANTDQDAPVATNNPARAVWEETRDRFYLDGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 549 aa, synthetic construct
SKLLTMQSSVTVQEGLCVHVPCSFSYPDHGTIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 549 aa, synthetic construct
SKLLTMQSSVTVQEGLCVHVPCSFSYPDHGEIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 549 aa, synthetic construct
SKLLTMQSSVTVQEGLCVHVPCSFSYPSHGSHHPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 549 aa, synthetic construct
SKLLTMQSSVTVQEGLCVHVPCSFSYPDHGQHEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 549 aa, synthetic construct
SKLLTMQSSVTVQEGLCVHVPCSFSYPTHGSHEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 549 aa, synthetic construct
SKLLTMQSSVTVQEGLCVHVPCSFSYPDHGETYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 549 aa, synthetic construct
SKLLTMQSSVTVQEGLCVHVPCSFSYPNHGTEYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 549 aa, synthetic construct
SKLLTMQSSVTVQEGLCVHVPCSFSYPHHGTTTPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 549 aa, synthetic construct
SKLLTMQSSVTVQEGLCVHVPCSFSYPHHGSTTPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 549 aa, synthetic construct
SKLLTMQSSVTVQEGLCVHVPCSFSYPSHGGTEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 549 aa, synthetic construct
DYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 549 aa, synthetic construct
DYQLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKNNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 549 aa, synthetic construct
EYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 549 aa, synthetic construct
SKLLTMQSSVTVQEGLCVHVPCSFSYPSHGQTDSGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 549 aa, synthetic construct
SKLLTMQSSVTVQEGLCVHVPCSFSYPSHSQTDSGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 549 aa, synthetic construct
SKLLTMQSSVTVQEGLCVHVPCSFSYPVDSQTDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 549 aa, synthetic construct
SKLLTMQSSVTVQEGLCVHVPCSFSYPVHGQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 549 aa, synthetic construct
SKLLTMQSSVTVQEGLCVHVPCSFSYPVHSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 549 aa, synthetic construct
SKLLTMQSSVTVQEGLCVHVPCSFSYPVDSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 549 aa, synthetic construct
SKLLTMQSSVTVQEGLCVHVPCSFSYPSDSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 549 aa, synthetic construct
SKLLTMQSSVTVQEGLCVHVPCSFSYPVDGQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 586 aa, synthetic construct
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 586 aa, synthetic construct
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 586 aa, synthetic construct
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGDIEGRMDPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 576 aa, synthetic construct
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGDIEGRMDCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 44 - protein, 587 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGGGGGSIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 45 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 577 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 568 aa, synthetic construct
MGWSCIILFLVATATGVHSSKLLTMQSSVTVQEGLCVHVPCSFSYPSHGDIEGGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 568 aa, synthetic construct
MGWSCIILFLVATATGVHSSKLLTMQSSVTVQEGLCVHVPCSFSYPSHGSIETGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 568 aa, synthetic construct
MGWSCIILFLVATATGVHSSKLLTMQSSVTVQEGLCVHVPCSFSYPSHGSIEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 568 aa, synthetic construct
MGWSCIILFLVATATGVHSSKLLTMQSSVTVQEGLCVHVPCSFSYPSHGDIEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 568 aa, synthetic construct
MGWSCIILFLVATATGVHSSKLLTMQSSVTVQEGLCVHVPCSFSYPSHGYQESGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 568 aa, synthetic construct
MGWSCIILFLVATATGVHSSKLLTMQSSVTVQEGLCVHVPCSFSYPSHGTHETGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 568 aa, synthetic construct
MGWSCIILFLVATATGVHSSKLLTMQSSVTVQEGTCVSVPCSFSYPSHGWIYPGPVVHGYWFREGANTDQDAPVATNNPARAVWEETRDRFYLEGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 568 aa, synthetic construct
MGWSCIILFLVATATGVHSSKLLTMQSSVTVQEGTCVTVPCSFSYPSHGWIYPGPVVHGYWFREGANTDQDAPVATNNPARAVWEETRDRFYLDGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 568 aa, synthetic construct
MGWSCIILFLVATATGVHSSKLLTMQSSVTVQEGLCVHVPCSFSYPDHGTIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 568 aa, synthetic construct
MGWSCIILFLVATATGVHSSKLLTMQSSVTVQEGLCVHVPCSFSYPDHGEIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 568 aa, synthetic construct
MGWSCIILFLVATATGVHSSKLLTMQSSVTVQEGLCVHVPCSFSYPSHGSHHPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 568 aa, synthetic construct
MGWSCIILFLVATATGVHSSKLLTMQSSVTVQEGLCVHVPCSFSYPDHGQHEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 568 aa, synthetic construct
MGWSCIILFLVATATGVHSSKLLTMQSSVTVQEGLCVHVPCSFSYPTHGSHEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 568 aa, synthetic construct
MGWSCIILFLVATATGVHSSKLLTMQSSVTVQEGLCVHVPCSFSYPDHGETYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 568 aa, synthetic construct
MGWSCIILFLVATATGVHSSKLLTMQSSVTVQEGLCVHVPCSFSYPNHGTEYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 568 aa, synthetic construct
MGWSCIILFLVATATGVHSSKLLTMQSSVTVQEGLCVHVPCSFSYPHHGTTTPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 568 aa, synthetic construct
MGWSCIILFLVATATGVHSSKLLTMQSSVTVQEGLCVHVPCSFSYPHHGSTTPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 568 aa, synthetic construct
MGWSCIILFLVATATGVHSSKLLTMQSSVTVQEGLCVHVPCSFSYPSHGGTEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 568 aa, synthetic construct
MGWSCIILFLVATATGVHSDYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 568 aa, synthetic construct
MGWSCIILFLVATATGVHSDYQLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKNNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 568 aa, synthetic construct
MGWSCIILFLVATATGVHSEYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 568 aa, synthetic construct
MGWSCIILFLVATATGVHSSKLLTMQSSVTVQEGLCVHVPCSFSYPSHGQTDSGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 568 aa, synthetic construct
MGWSCIILFLVATATGVHSSKLLTMQSSVTVQEGLCVHVPCSFSYPSHSQTDSGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 568 aa, synthetic construct
MGWSCIILFLVATATGVHSSKLLTMQSSVTVQEGLCVHVPCSFSYPVDSQTDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 568 aa, synthetic construct
MGWSCIILFLVATATGVHSSKLLTMQSSVTVQEGLCVHVPCSFSYPVHGQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 568 aa, synthetic construct
MGWSCIILFLVATATGVHSSKLLTMQSSVTVQEGLCVHVPCSFSYPVHSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 568 aa, synthetic construct
MGWSCIILFLVATATGVHSSKLLTMQSSVTVQEGLCVHVPCSFSYPVDSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 568 aa, synthetic construct
MGWSCIILFLVATATGVHSSKLLTMQSSVTVQEGLCVHVPCSFSYPSDSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 568 aa, synthetic construct
MGWSCIILFLVATATGVHSSKLLTMQSSVTVQEGLCVHVPCSFSYPVDGQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 79 - protein, 317 aa, synthetic construct
SKLLTMQSSVTVQEGLCVHVPCSFSYPSHGDIEGGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 80 - protein, 317 aa, synthetic construct
SKLLTMQSSVTVQEGLCVHVPCSFSYPSHGSIETGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 81 - protein, 317 aa, synthetic construct
SKLLTMQSSVTVQEGLCVHVPCSFSYPSHGSIEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 82 - protein, 317 aa, synthetic construct
SKLLTMQSSVTVQEGLCVHVPCSFSYPSHGDIEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 83 - protein, 317 aa, synthetic construct
SKLLTMQSSVTVQEGLCVHVPCSFSYPSHGYQESGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 84 - protein, 317 aa, synthetic construct
SKLLTMQSSVTVQEGLCVHVPCSFSYPSHGTHETGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 85 - protein, 317 aa, synthetic construct
SKLLTMQSSVTVQEGTCVSVPCSFSYPSHGWIYPGPVVHGYWFREGANTDQDAPVATNNPARAVWEETRDRFYLEGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 86 - protein, 317 aa, synthetic construct
SKLLTMQSSVTVQEGTCVTVPCSFSYPSHGWIYPGPVVHGYWFREGANTDQDAPVATNNPARAVWEETRDRFYLDGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 87 - protein, 317 aa, synthetic construct
SKLLTMQSSVTVQEGLCVHVPCSFSYPDHGTIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 88 - protein, 317 aa, synthetic construct
SKLLTMQSSVTVQEGLCVHVPCSFSYPDHGEIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 89 - protein, 317 aa, synthetic construct
SKLLTMQSSVTVQEGLCVHVPCSFSYPSHGSHHPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 90 - protein, 317 aa, synthetic construct
SKLLTMQSSVTVQEGLCVHVPCSFSYPDHGQHEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 91 - protein, 317 aa, synthetic construct
SKLLTMQSSVTVQEGLCVHVPCSFSYPTHGSHEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 92 - protein, 317 aa, synthetic construct
SKLLTMQSSVTVQEGLCVHVPCSFSYPDHGETYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 93 - protein, 317 aa, synthetic construct
SKLLTMQSSVTVQEGLCVHVPCSFSYPNHGTEYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 94 - protein, 317 aa, synthetic construct
SKLLTMQSSVTVQEGLCVHVPCSFSYPHHGTTTPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 95 - protein, 317 aa, synthetic construct
SKLLTMQSSVTVQEGLCVHVPCSFSYPHHGSTTPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 96 - protein, 317 aa, synthetic construct
SKLLTMQSSVTVQEGLCVHVPCSFSYPSHGGTEPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 97 - protein, 317 aa, synthetic construct
DYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 98 - protein, 317 aa, synthetic construct
DYQLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKNNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 99 - protein, 317 aa, synthetic construct
EYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 100 - protein, 317 aa, synthetic construct
SKLLTMQSSVTVQEGLCVHVPCSFSYPSHGQTDSGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 101 - protein, 317 aa, synthetic construct
SKLLTMQSSVTVQEGLCVHVPCSFSYPSHSQTDSGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 102 - protein, 317 aa, synthetic construct
SKLLTMQSSVTVQEGLCVHVPCSFSYPVDSQTDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 103 - protein, 317 aa, synthetic construct
SKLLTMQSSVTVQEGLCVHVPCSFSYPVHGQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 104 - protein, 317 aa, synthetic construct
SKLLTMQSSVTVQEGLCVHVPCSFSYPVHSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 105 - protein, 317 aa, synthetic construct
SKLLTMQSSVTVQEGLCVHVPCSFSYPVDSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 106 - protein, 317 aa, synthetic construct
SKLLTMQSSVTVQEGLCVHVPCSFSYPSDSQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 107 - protein, 317 aa, synthetic construct
SKLLTMQSSVTVQEGLCVHVPCSFSYPVDGQIDSDPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 108 - protein, 121 aa, synthetic construct; identical to SEQ ID NO: 7

SEQ ID NO: 109 - protein, 121 aa, synthetic construct
SKLLTMQSSVTVQEGLCVHVPCSFSYPSHGDIEGGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 110 - protein, 121 aa, synthetic construct
SKLLTMQSSVTVQEGLCVHVPCSFSYPSHGSIETGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 111 - protein, 121 aa, synthetic construct
SKLLTMQSSVTVQEGLCVHVPCSFSYPSHGSIEPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 112 - protein, 121 aa, synthetic construct
SKLLTMQSSVTVQEGLCVHVPCSFSYPSHGDIEPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 113 - protein, 121 aa, synthetic construct
SKLLTMQSSVTVQEGLCVHVPCSFSYPSHGYQESGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 114 - protein, 121 aa, synthetic construct
SKLLTMQSSVTVQEGLCVHVPCSFSYPSHGTHETGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 115 - protein, 121 aa, synthetic construct
SKLLTMQSSVTVQEGTCVSVPCSFSYPSHGWIYPGPVVHGYWFREGANTDQDAPVATNNPARAVWEETRDRFYLEGDPHTKNCTLSIRDARRSDAGRYFFRMEKGSIKWNYKHHRLSVNVT

SEQ ID NO: 116 - protein, 121 aa, synthetic construct
SKLLTMQSSVTVQEGTCVTVPCSFSYPSHGWIYPGPVVHGYWFREGANTDQDAPVATNNPARAVWEETRDRFYLDGDPHTKNCTLSIRDARRSDAGRYFFRMEKGSIKWNYKHHRLSVNVT

SEQ ID NO: 117 - protein, 121 aa, synthetic construct
SKLLTMQSSVTVQEGLCVHVPCSFSYPDHGTIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 118 - protein, 121 aa, synthetic construct
SKLLTMQSSVTVQEGLCVHVPCSFSYPDHGEIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 119 - protein, 121 aa, synthetic construct
SKLLTMQSSVTVQEGLCVHVPCSFSYPSHGSHHPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 120 - protein, 121 aa, synthetic construct
SKLLTMQSSVTVQEGLCVHVPCSFSYPDHGQHEPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 121 - protein, 121 aa, synthetic construct
SKLLTMQSSVTVQEGLCVHVPCSFSYPTHGSHEPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 122 - protein, 121 aa, synthetic construct
SKLLTMQSSVTVQEGLCVHVPCSFSYPDHGETYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 123 - protein, 121 aa, synthetic construct
SKLLTMQSSVTVQEGLCVHVPCSFSYPNHGTEYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 124 - protein, 121 aa, synthetic construct
SKLLTMQSSVTVQEGLCVHVPCSFSYPHHGTTTPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 125 - protein, 121 aa, synthetic construct
SKLLTMQSSVTVQEGLCVHVPCSFSYPHHGSTTPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 126 - protein, 121 aa, synthetic construct
SKLLTMQSSVTVQEGLCVHVPCSFSYPSHGGTEPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 127 - protein, 121 aa, synthetic construct
DYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVT

SEQ ID NO: 128 - protein, 121 aa, synthetic construct
DYQLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKNNYKHHRLSVNVT

SEQ ID NO: 129 - protein, 121 aa, synthetic construct
EYTLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKENYKHHRLSVNVT

SEQ ID NO: 130 - protein, 121 aa, synthetic construct
SKLLTMQSSVTVQEGLCVHVPCSFSYPSHGQTDSGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 131 - protein, 121 aa, synthetic construct
SKLLTMQSSVTVQEGLCVHVPCSFSYPSHSQTDSGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 132 - protein, 121 aa, synthetic construct
SKLLTMQSSVTVQEGLCVHVPCSFSYPVDSQTDSDPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 133 - protein, 121 aa, synthetic construct
SKLLTMQSSVTVQEGLCVHVPCSFSYPVHGQIDSDPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 134 - protein, 121 aa, synthetic construct
SKLLTMQSSVTVQEGLCVHVPCSFSYPVHSQIDSDPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 135 - protein, 121 aa, synthetic construct
SKLLTMQSSVTVQEGLCVHVPCSFSYPVDSQIDSDPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 136 - protein, 121 aa, synthetic construct
SKLLTMQSSVTVQEGLCVHVPCSFSYPSDSQIDSDPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 137 - protein, 121 aa, synthetic construct
SKLLTMQSSVTVQEGLCVHVPCSFSYPVDGQIDSDPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 138 - protein, 348 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQG

SEQ ID NO: 139 - protein, 586 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 19 aa, synthetic construct
MLLLLLPLLWGRERAEGQT

SEQ ID NO: 141 - protein, 19 aa, synthetic construct
MGWSCIILFLVATATGVHS

SEQ ID NO: 142 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 12 aa, synthetic construct
LQSKATSGVTQG

SEQ ID NO: 148 - protein, 549 aa, synthetic construct
SKLLTMQSSVTVQEGLCVHVPCSFSYPSHGTIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 549 aa, synthetic construct
SKLLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 549 aa, synthetic construct
SKLLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 549 aa, synthetic construct
SKLLTMQSSVTVQEGLCVHVPCSFSYPSHGAIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 549 aa, synthetic construct
SKLLTMQSSVTVQEGLCVHVPCSFSYPSHGRIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 549 aa, synthetic construct
SKLLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 549 aa, synthetic construct
SKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 549 aa, synthetic construct
SKLLTMQSSVTVQEGLCVHVPCSFSYPSHGSITPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 549 aa, synthetic construct
SKLLTMQSSVTVQEGLCVHVPCSFSYPSHGERYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 549 aa, synthetic construct
SKDLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 549 aa, synthetic construct
SKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 549 aa, synthetic construct
SKTLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 549 aa, synthetic construct
SKSLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSSSPLDPSTTRSSVLTLIPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSDSPLDPSTTRSSVLTLIPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVTPLDPSTTRSSVLTLQPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESNCPQNLTCSVPWACEQGTPPMISWIGTSTTPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVKPLDPSTTRSSVLTLIPEPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSSSPLDPSTTRSSVLTLQPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSSKPLDPSTTRSSVLTLQPEPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 549 aa, synthetic construct
SKLLTMQSSVTVQEGLCVHVPCSFSYPSHGRIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 549 aa, synthetic construct
SKLLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 549 aa, synthetic construct
SKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 568 aa, synthetic construct
MGWSCIILFLVATATGVHSSKLLTMQSSVTVQEGLCVHVPCSFSYPSHGTIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 568 aa, synthetic construct
MGWSCIILFLVATATGVHSSKLLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 568 aa, synthetic construct
MGWSCIILFLVATATGVHSSKLLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 568 aa, synthetic construct
MGWSCIILFLVATATGVHSSKLLTMQSSVTVQEGLCVHVPCSFSYPSHGAIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 568 aa, synthetic construct
MGWSCIILFLVATATGVHSSKLLTMQSSVTVQEGLCVHVPCSFSYPSHGRIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 568 aa, synthetic construct
MGWSCIILFLVATATGVHSSKLLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 568 aa, synthetic construct
MGWSCIILFLVATATGVHSSKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 568 aa, synthetic construct
MGWSCIILFLVATATGVHSSKLLTMQSSVTVQEGLCVHVPCSFSYPSHGSITPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 568 aa, synthetic construct
MGWSCIILFLVATATGVHSSKLLTMQSSVTVQEGLCVHVPCSFSYPSHGERYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 568 aa, synthetic construct
MGWSCIILFLVATATGVHSSKDLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 568 aa, synthetic construct
MGWSCIILFLVATATGVHSSKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 568 aa, synthetic construct
MGWSCIILFLVATATGVHSSKTLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 568 aa, synthetic construct
MGWSCIILFLVATATGVHSSKSLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSSSPLDPSTTRSSVLTLIPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSDSPLDPSTTRSSVLTLIPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVTPLDPSTTRSSVLTLQPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESNCPQNLTCSVPWACEQGTPPMISWIGTSTTPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVKPLDPSTTRSSVLTLIPEPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSSSPLDPSTTRSSVLTLQPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSSKPLDPSTTRSSVLTLQPEPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 568 aa, synthetic construct
MGWSCIILFLVATATGVHSSKLLTMQSSVTVQEGLCVHVPCSFSYPSHGRIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 568 aa, synthetic construct
MGWSCIILFLVATATGVHSSKLLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 568 aa, synthetic construct
MGWSCIILFLVATATGVHSSKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 317 aa, synthetic construct
SKLLTMQSSVTVQEGLCVHVPCSFSYPSHGTIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 195 - protein, 317 aa, synthetic construct
SKLLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 196 - protein, 317 aa, synthetic construct
SKLLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 197 - protein, 317 aa, synthetic construct
SKLLTMQSSVTVQEGLCVHVPCSFSYPSHGAIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 198 - protein, 317 aa, synthetic construct
SKLLTMQSSVTVQEGLCVHVPCSFSYPSHGRIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 199 - protein, 317 aa, synthetic construct
SKLLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 200 - protein, 317 aa, synthetic construct
SKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 201 - protein, 317 aa, synthetic construct
SKLLTMQSSVTVQEGLCVHVPCSFSYPSHGSITPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 202 - protein, 317 aa, synthetic construct
SKLLTMQSSVTVQEGLCVHVPCSFSYPSHGERYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 203 - protein, 317 aa, synthetic construct
SKDLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 204 - protein, 317 aa, synthetic construct
SKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 205 - protein, 317 aa, synthetic construct
SKTLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKENYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 206 - protein, 317 aa, synthetic construct
SKSLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKRNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 207 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSSSPLDPSTTRSSVLTLIPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 208 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSDSPLDPSTTRSSVLTLIPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 209 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVTPLDPSTTRSSVLTLQPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 210 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESNCPQNLTCSVPWACEQGTPPMISWIGTSTTPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 211 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVKPLDPSTTRSSVLTLIPEPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 212 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESRCPQNLTCSVPWACEQGTPPMISWIGTSSSPLDPSTTRSSVLTLQPTPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 213 - protein, 317 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSSKPLDPSTTRSSVLTLQPEPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVS

SEQ ID NO: 214 - protein, 121 aa, synthetic construct
SKLLTMQSSVTVQEGLCVHVPCSFSYPSHGTIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 215 - protein, 121 aa, synthetic construct
SKLLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 216 - protein, 121 aa, synthetic construct
SKLLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 217 - protein, 121 aa, synthetic construct
SKLLTMQSSVTVQEGLCVHVPCSFSYPSHGAIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 218 - protein, 121 aa, synthetic construct
SKLLTMQSSVTVQEGLCVHVPCSFSYPSHGRIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 219 - protein, 121 aa, synthetic construct
SKLLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 220 - protein, 121 aa, synthetic construct
SKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 221 - protein, 121 aa, synthetic construct
SKLLTMQSSVTVQEGLCVHVPCSFSYPSHGSITPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 222 - protein, 121 aa, synthetic construct
SKLLTMQSSVTVQEGLCVHVPCSFSYPSHGERYPGPVVHGYWFREGANTDQDAPVATNNPARAVWEETRDRFHLLGDPHTKNCTLSIRDARRSDAGRYFFRMEKGSIKRNYKHHRLSVNVT

SEQ ID NO: 223 - protein, 121 aa, synthetic construct
SKDLTMQSSVTVQEGLCVHVPCSFSYPSHGSIYPGPVVHGYWFREGANTDQDAPVATNNPARAVWEETRDRFHLLGDPHTKNCTLSIRDARRSDAGRYFFRMEKGSIKRNYKHHRLSVNVT

SEQ ID NO: 224 - protein, 121 aa, synthetic construct
SKLLTMQSSVTVQEGLCVHVPCSFSYPSHGKIYPGPVVHGYWFREGANTDQDAPVATNNPARAVWEETRDRFHLLGDPHTKNCTLSIRDARRSDAGRYFFRMEKGSIKRNYKHHRLSVNVT

SEQ ID NO: 225 - protein, 121 aa, synthetic construct
SKTLTMQSSVTVQEGLCVHVPCSFSYPSHGEIYPGPVVHGYWFREGANTDQDAPVATNNPARAVWEETRDRFHLLGDPHTKNCTLSIRDARRSDAGRYFFRMEKGSIKENYKHHRLSVNVT

SEQ ID NO: 226 - protein, 121 aa, synthetic construct
SKSLTMQSSVTVQEGLCVHVPCSFSYPSHGQIYPGPVVHGYWFREGANTDQDAPVATNNPARAVWEETRDRFHLLGDPHTKNCTLSIRDARRSDAGRYFFRMEKGSIKRNYKHHRLSVNVT

SEQ ID NO: 227 - protein, 549 aa, synthetic construct
SKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 230 - protein, 580 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGGCALYPTNCGGGQPENNYKTTPPVLDSDGSFFLYSKLTVDKSRWQQGNVFSCSVMHEALHNHYTQKSLSLSPGK

SEQ ID NO: 231 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 568 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 233 - protein, 580 aa, synthetic construct
MGWSCIILFLVATATGVHSSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGGCALYPTNCGGGQPENNYKTTPPVLDSDGSFFLYSKLTVDKSRWQQGNVFSCSVMHEALHNHYTQKSLSLSPGK

SEQ ID NO: 234 - protein, 232 aa, synthetic construct
EPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 236 - protein, 244 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGGCALYPTNCGGGQPENNYKTTPPVLDSDGSFFLYSKLTVDKSRWQQGNVFSCSVMHEALHNHYTQKSLSLSPGK

SEQ ID NO: 237 - protein, 232 aa, synthetic construct
EPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 239 - protein, 244 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGGCALYPTNCGGGQPENNYKTTPPVLDSDGSFFLYSKLTVDKSRWQQGNVFSCSVMHEALHNHYTQKSLSLSPGK

SEQ ID NO: 240 - protein, 11 aa, synthetic construct
RASQEISGYLG

SEQ ID NO: 241 - protein, 7 aa, synthetic construct
STSTLDS

SEQ ID NO: 242 - protein, 9 aa, synthetic construct
LQYASYPPT

SEQ ID NO: 243 - protein, 9 aa, synthetic construct
FTFSNYAMS

SEQ ID NO: 244 - protein, 14 aa, synthetic construct
VATINNGGSYTYYS

SEQ ID NO: 245 - protein, 12 aa, synthetic construct
VRRDYGTSDFDY

SEQ ID NO: 246 - protein, 107 aa, synthetic construct
DIQMTQSPSSLSASLGERVSLTCRASQEISGYLGWLQQKPDGTIKRLIFSTSTLDSGVPKRFSGSRSGSDYSLTISSLESEDFADYYCLQYASYPPTFGGGTKLEIK

SEQ ID NO: 247 - protein, 119 aa, synthetic construct
EVKLVESGGALVKPGGSLKLSCAASGFTFSNYAMSWVRQTPEKRLEWVATINNGGSYTYYSDSVKGRFAISRDNAKNTLYLQMSNLRSEDTALYYCVRRDYGTSDFDYWGQGTTLTVSS

SEQ ID NO: 248 - protein, 4 aa, synthetic construct
DIEG

SEQ ID NO: 249 - protein, 4 aa, synthetic construct
SIET

SEQ ID NO: 250 - protein, 4 aa, synthetic construct
SIEP

SEQ ID NO: 251 - protein, 4 aa, synthetic construct
DIEP

SEQ ID NO: 252 - protein, 4 aa, synthetic construct
YQES

SEQ ID NO: 253 - protein, 4 aa, synthetic construct
THET

SEQ ID NO: 254 - protein, 4 aa, synthetic construct
WIYP

SEQ ID NO: 255 - protein, 4 aa, synthetic construct
QTDS

SEQ ID NO: 256 - protein, 5 aa, synthetic construct
GWIYP

SEQ ID NO: 257 - protein, 5 aa, synthetic construct
SQTDS

SEQ ID NO: 258 - protein, 8 aa, synthetic construct
VHGQIDSD

SEQ ID NO: 259 - protein, 8 aa, synthetic construct
VHSQIDSD

SEQ ID NO: 260 - protein, 8 aa, synthetic construct
VDSQIDSD

SEQ ID NO: 261 - protein, 8 aa, synthetic construct
SDSQIDSD

SEQ ID NO: 262 - protein, 8 aa, synthetic construct
VDGQIDSD

SEQ ID NO: 263 - protein, 4 aa, synthetic construct
LERR

SEQ ID NO: 264 - protein, 4 aa, synthetic construct
DSIR

SEQ ID NO: 265 - protein, 4 aa, synthetic construct
LKIR

SEQ ID NO: 266 - protein, 4 aa, synthetic construct
TEIE

SEQ ID NO: 267 - protein, 4 aa, synthetic construct
SQIR

SEQ ID NO: 268 - protein, 5 aa, synthetic construct
RSSIT

SEQ ID NO: 269 - protein, 5 aa, synthetic construct
RDSIT

SEQ ID NO: 270 - protein, 5 aa, synthetic construct
GVTQT

SEQ ID NO: 271 - protein, 5 aa, synthetic construct
NTTIQ

SEQ ID NO: 272 - protein, 5 aa, synthetic construct
GVKIE

SEQ ID NO: 273 - protein, 5 aa, synthetic construct
RSSQT

SEQ ID NO: 274 - protein, 5 aa, synthetic construct
GSKQE